(19) United States
(12) Patent Application Publication
(10) Pub. No.: US 2021/0381007 A1
(43) Pub. Date: Dec. 9, 2021

(54) METHODS AND COMPOSITIONS FOR CRISPR EDITING OF CELLS AND CORRELATING THE EDITS TO A RESULTING CELLULAR NUCLEIC ACID PROFILE

(71) Applicant: Inscripta, Inc., Boulder, CO (US)

(72) Inventors: Janine Mok, Boulder, CO (US); Miles Gander, Boulder, CO (US); Andrew Garst, Boulder, CO (US); Burak Dura, Boulder, CO (US); Deanna Church, Boulder, CO (US)

(21) Appl. No.: 17/338,603

(22) Filed: Jun. 3, 2021

Related U.S. Application Data
(60) Provisional application No. 63/034,491, filed on Jun. 4, 2020.

(51) Int. Cl. C12N 15/90; C12N 15/10; C12N 15/11; C12N 9/22; C12Q 1/6869

(52) U.S. Cl. CPC C12N 15/905; C12N 15/1065; C12N 15/907; C12N 2800/80; C12N 9/22; C12Q 1/6869; C12N 2310/20; C12N 15/11

(57) ABSTRACT

The present disclosure provides compositions, methods and modules to edit live cells and to subsequently correlate the resulting cellular nucleic acids of the edited cells to the edits.

### RELATED CASES

This application claims priority to U.S. Ser. No. 63/034,491, filed 4 Jun. 2020, entitled “Compositions, Methods, Modules and Instruments for Automated Nucleic Acid-Guided Nuclease Editing in Mammalian Cells”, which is incorporated herein in its entirety.

### FIELD OF THE INVENTION

The present disclosure relates to methods, compositions and modules to perform nucleic acid-guided editing in live cells, determine a nucleic acid profile of the edited cells and correlate the edits to the resulting cellular transcriptome, cell surface protein profile, intracellular protein profile, cellular nucleic acid methylation profile and/or chromatin accessibility profile.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The ability to make precise, targeted changes to the genome of living cells has been a long-standing goal in biomedical research and development. Recently, various nucleases have been identified that allow for manipulation of gene sequences, and hence gene function. The nucleases include nucleic acid-guided nucleases, which enable researchers to generate permanent edits in live cells. Of course, it is desirable to determine the cellular consequences of the edit(s) by, e.g., analyzing changes to the cellular transcriptome, cell surface protein expression, intracellular protein expression, genomic methylation patterns and/or chromatin accessibility after the edits have been made, particularly in a multiplex format.

There is thus a need in the art of nucleic acid-guided nuclease editing for improved methods, compositions, modules and instruments for determining the cellular consequences of editing. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to methods, compositions and modules that allow correlation between an editor change to a cellular genome and the cellular consequences resulting from that change. Pairing nucleic acid-guided nuclease or nickase fusion edits with single-cell phenotypes has allowed for, e.g., exploration of the function of genes and biological pathways, gene regulation and protein expression. Typically, however, methods for correlating edits with phenotype take place in either an individual arrayed format, where each editing cassette is delivered and assessed separately, or a pooled format, where a single editing cassette is delivered to a population of cells and the resulting phenotype is determined from the population of edited cells. The present methods allow for multiplex editing in a large population of cells using a library of different editing vectors where each editing vector comprises a different editing cassette. Once the edits have taken place, the present methods allow for correlating the resulting edit(s) to the resulting cellular phenotype in individual cells.

The present methods and compositions utilize capture primers to 1) capture the editing cassettes, which identifies the edit(s) made to the cells; and 2) to capture the nucleic acids, proteins, methylated nucleic acids and/or accessible nucleic acids (collectively, “products”) that result from the edit(s). The cassette capture primers comprise a capture sequence that is specific for the editing cassette (or one or more barcodes that serve as a proxy for an editing cassette) in each cell. The product capture primers comprise a capture sequence that is specific for the products resulting from the edit (or for a tag or barcode that serves as a proxy for the resulting products). In almost all cases, the capture primers also comprise a primer sequence, which allows the captured cassettes or captured products (or proxies thereof) to be amplified. Thus, the present method and composition embodiments utilize both cassette capture primers and product capture primers where the cassette capture primers and product capture primers may be separate molecules (see, FIGS. 6A-6F) or the cassette capture primers and product capture primers may be covalently linked on a single molecule. The terms “cassette capture primer” and “product capture primer” are defined infra.

The present methods use a combination of 1) a barcoded cassette capture primer to capture the editing cassette; and 2) a barcoded product capture primer to capture nucleic acids, proteins and other cellular analytes from individual cells. In some embodiments, the barcoded cassette capture primer is a combination of a cassette capture primer and a barcoded template switching oligonucleotide and the barcoded product capture primer is a combination of a product capture primer and a barcoded template switching oligonucleotide (see, e.g., FIGS. 6A-6F). The methods use various combinations of one or more of the processes of priming, reverse transcription or transcription, extension and amplification. Once the editing cassettes and products from each cell are captured, barcoded and amplified, the nucleic acids from each cell can be pooled, sequenced, and the editing cassettes associated with the type and level of products present in a cell.

While other platforms and other single cell RNA approaches facilitate study of, e.g., the transcriptome on a per cell basis, until the present disclosure there were no platforms that combine the ability to make a precise edit and then correlate that edit with effects on the cellular nucleic acids. A related protocol involves the use of Cas9 gRNA-mediated knockouts. In this method, the gRNAs are barcoded and the effect of a gene knockout can be correlated with transcriptome; however, this protocol does not have the ability to correlate precise edits with changes in the nucleic acid profile of individual cells in a multiplex fashion. Other features that distinguish the present methods include increasing the specificity of the editing cassettes captured by, e.g., performing hybrid capture of the editing cassettes, including primer-added specificity sequences on the editing cassette, as well as general workflow optimization. In addition, certain of the present methods have been adapted for bacterial cells, yeast cells and mammalian cells, while the other platforms are only designed for mammalian cells. In addition, the methods herein include methods for nested PCR or hybrid capture of DNA products (e.g., DNA copies of nucleic acids or cDNAs of RNAs).

Thus, there is provided in one embodiment a method for correlating rationally-designed genome edits made in a population of cells with a cellular nucleic acid profile from individual cells comprising: designing and synthesizing a library of editing cassettes comprising repair templates and gRNAs; inserting the library of editing cassettes in a vector backbone resulting in a library of editing vectors; transforming the population of cells with the library of editing vectors to produce transformed cells; allowing editing to take place in the transformed cells to produce edited cells; singulating the edited cells into partitions; lysing the edited cells; adding barcoded random capture primers (in this embodiment, the product capture primers are random capture primers) and barcoded cassette capture primers to each partition, wherein the barcodes used in the barcoded random capture primers and the barcoded cassette capture primers in a same partition are the same barcode and wherein the barcodes used in the barcoded random capture primers and the barcoded cassette capture primers in a different partition are different from barcodes used in other partitions; creating DNA copies or cDNAs from cellular nucleic acids in the edited cells using the barcoded random capture primers; creating DNA copies or cDNAs from the editing cassettes in the edited cells using the barcoded cassette capture primers; pooling the nucleic acids from the partitions; sequencing the nucleic acids; and correlating the nucleic acid sequences from the cellular nucleic acids with nucleic acid sequences from the editing cassettes for each cell. It should be apparent to one of ordinary skill given the present disclosure in the art that if the product capture primers and cassette capture primers are located on a single molecule only a single barcode is necessary; that is, both the product capture primers and cassette capture primers need not comprise a separate barcode.

Additionally, there is provided in one embodiment a method for correlating rationally-designed genome edits made in a population of cells with mRNA profiles from the individual cells comprising: designing and synthesizing a library of editing cassettes comprising repair templates and gRNAs; inserting the library of editing cassettes in a vector backbone resulting in a library of editing vectors; transforming the population of cells with the library of editing vectors to produce transformed cells; allowing editing to take place in the transformed cells to produce edited cells; singulating the edited cells into partitions; lysing the edited cells; adding barcoded poly-dT capture primers and barcoded cassette capture primers to each partition, wherein the barcodes used in the barcoded poly-dT capture primers and the barcoded cassette capture primers in a same partition are the same barcode and wherein the barcodes used in the barcoded poly-dT capture primers and the barcoded cassette capture primers in a different partition are different from barcodes used in other partitions; creating cDNAs from the mRNAs in the edited cells using the barcoded poly-dT capture primers; creating DNA copies or cDNAs from the editing cassettes in the edited cells using the barcoded cassette capture primers; pooling the nucleic acids from the partitions; sequencing the nucleic acids; and correlating the nucleic acid sequences from cellular nucleic acids with the nucleic acid sequences from the editing cassettes for each cell.

Yet other embodiments provide a method for correlating rationally-designed genome edits made in a population of cells with a cell surface protein profile from individual cells comprising: designing and synthesizing a library of editing cassettes comprising repair templates and gRNAs; inserting the library of editing cassettes in a vector backbone resulting in a library of editing vectors; transforming the population of cells with the library of editing vectors to produce transformed cells; allowing editing to take place in the transformed cells to produce edited cells; adding nucleic acid-tagged antibodies against cell surface proteins to the edited cells, wherein each different antibody is tagged with a different nucleic acid tag and wherein some nucleic acid-tagged antibodies bind to the cell surface proteins in the edited cells; washing unbound nucleic acid-tagged antibodies from the edited cells; singulating the edited cells into partitions; lysing the edited cells; adding barcoded analyte capture primers (in this embodiment, the product capture primers are analyte capture primers, which are also sequence-specific primers) and barcoded cassette capture primers to each partition, wherein the barcodes used in the barcoded analyte capture primers and the barcoded cassette capture primers in the same partition are the same barcode and wherein the barcodes used in the barcoded analyte capture primers and the barcoded cassette capture primers in a different partition are different from barcodes used in other partitions; creating DNA copies or cDNAs from the nucleic acid tags on the bound antibodies using the barcoded analyte capture primers; creating DNA copies or cDNAs from the editing cassettes using the barcoded cassette capture primers; pooling the nucleic acids from the partitions; sequencing the nucleic acids; and correlating the nucleic acid sequences from the nucleic acid tags on the bound antibodies with the nucleic acid sequences from the editing cassettes for each cell.

Yet other embodiments provide a method for correlating rationally-designed genome edits made in a population of cells with an intracellular protein profile from individual cells comprising: designing and synthesizing a library of editing cassettes comprising repair templates and gRNAs; inserting the library of editing cassettes in a vector backbone resulting in a library of editing vectors; transforming the population of cells with the library of editing vectors to produce transformed cells; allowing editing to take place in the transformed cells to produce edited cells; fixing and permeabilizing the edited cells; adding nucleic acid-tagged antibodies against intracellular proteins to the fixed and permeabilized cells, wherein each different antibody is tagged with a different nucleic acid and wherein some nucleic acid-tagged antibodies bind to the intracellular proteins; washing unbound nucleic acid-tagged antibodies from the fixed and permeabilized cells; singulating the fixed and permeabilized cells into partitions; lysing the fixed and permeabilized cells to produce lysed cells; reversing cross-linking in the lysed cells; adding barcoded analyte capture primers (in this embodiment, the product capture primers are analyte capture primers and are also sequence-specific capture primers) and barcoded cassette capture primers to each partition, wherein the barcodes used in the barcoded analyte capture primers and the barcoded cassette capture primers in the same partition are the same barcode and wherein the barcodes used in the barcoded analyte capture primers and the barcoded cassette capture primers in a different partition are different from the barcodes used in other partitions; creating DNA copies or cDNAs from the nucleic acid tags on the bound antibodies using the barcoded analyte capture primers; creating DNA copies or cDNAs from the editing cassettes using the barcoded cassette capture primers; pooling the nucleic acids from the partitions; sequencing the nucleic acids; and correlating the nucleic acid sequences from the nucleic acid tags on the bound antibodies with the nucleic acid sequences from the editing cassettes for each cell.

Other embodiments provide a method for correlating rationally-designed genome edits made in a population of cells with a methylated cellular nucleic acid profile from individual cells comprising: designing and synthesizing a library of editing cassettes comprising repair templates and gRNAs; inserting the library of editing cassettes in a vector backbone resulting in a library of editing vectors; transforming the population of cells with the library of editing vectors to produce transformed cells; allowing editing to take place in the transformed cells to produce edited cells; singulating the edited cells into partitions; lysing the edited cells; conducting bisulfite conversion to convert unmethylated cytosine residues to uracil residues in the lysed cells; adding barcoded random capture primers (in this embodiment the product capture primers are random capture primers) and barcoded cassette capture primers to each partition, wherein the barcodes used in the barcoded random capture primers and the barcoded cassette capture primers in the same partition are the same barcode and wherein the barcodes used in the barcoded random capture primers and the barcoded cassette capture primers in a different partition are different from barcodes used in other partitions; creating DNA copies from cellular nucleic acids in the edited cells using the barcoded random capture primers; creating DNA copies or cDNAs from the editing cassettes in the edited cells using the barcoded cassette capture primers; pooling the nucleic acids from the partitions; sequencing the nucleic acids; correlating the nucleic acid sequences from cellular nucleic acids with the nucleic acid sequences from the editing cassettes; and comparing the nucleic acid sequences from cellular nucleic acids with a reference sequence to determine which cytosine residues in the cellular nucleic acids were converted to uracil residues for each cell.

Additional embodiments provide a method for correlating rationally-designed genome edits made in a population of cells with a chromatin accessibility profile from individual cells comprising: designing and synthesizing a library of editing cassettes comprising repair templates and gRNAs; inserting the library of editing cassettes in a vector backbone resulting in a library of editing vectors; transforming the population of cells with the library of editing vectors to produce transformed cells; allowing editing to take place in the transformed cells to produce edited cells; fixing the edited cells; singulating the edited cells into partitions; lysing the edited cells; subjecting the cells to MNase digestion; adding barcoded random capture molecules (in this embodiment, the product capture primers are random capture molecules; e.g., not primers, as the random capture molecules are ligated to cellular nucleic acids) and barcoded cassette capture primers to each partition, wherein the barcodes used in the barcoded random capture molecules and the barcoded cassette capture primers in the same partition are the same barcode and wherein the barcodes used in the barcoded random capture molecules and the barcoded cassette capture primers in a different partition are different from the barcodes used in other partitions; ligating the barcoded random capture molecules to the cellular nucleic acids; creating DNA copies or cDNAs from the editing cassettes in the edited cells using the barcoded cassette capture primers; pooling the nucleic acids from the partitions; adding histone antibodies to the pool of nucleic acids; allowing the histone antibodies to bind to histones in the nucleic acids; immunoprecipitating the histone antibodies bound to histones in the nucleic acids resulting in immunoprecipitated nucleic acids and un-immunoprecipitated nucleic acids; separating the immunoprecipitated nucleic acids from un-immunoprecipitated nucleic acids; reversing the cross-linking and amplifying the barcoded immunoprecipitated DNA; sequencing the separated nucleic acids, resulting in histone-associated nucleic acid sequences and non-histone-associated nucleic acid sequences, wherein the non-histone-associated nucleic acid sequences include the nucleic acids from the editing cassettes; and correlating sequences from the histone-associated nucleic acids with sequences from the non-histone-associated nucleic acids from the editing cassettes. Note that the principles behind chromatin immunoprecipitation may be extended to look at other protein nucleic acid associations other than histones (e.g., transcription factor binding using TF-specific antibodies, methylation using antibodies to methyl-cytosine residues, etc.

Another embodiment provides a method for correlating rationally-designed genome edits made in a population of cells with a chromatin accessibility profile from individual cells comprising: designing and synthesizing a library of editing cassettes comprising repair templates and gRNAs; inserting the library of editing cassettes in a vector backbone resulting in a library of editing vectors; transforming the population of cells with the library of editing vectors to produce transformed cells; allowing editing to take place in the transformed cells to produce edited cells; singulating the edited cells into partitions; lysing the edited cells; subjecting the cells to a TN5 transposon assay including adapters, wherein adapters are inserted where chromatin is accessible and wherein the adapters optionally may include barcodes and one or more priming sequences; adding the barcoded adapter capture primers (in this embodiment the product capture primers are adapter capture primers, which are also sequence-specific primers) and the barcoded cassette capture primers to each partition, wherein the barcodes used in the barcoded adapter capture primers and barcoded cassette capture primers in the same partition are the same barcode and wherein the barcodes used in the barcoded adapter capture primers and barcoded cassette capture primers in a different partition are different from barcodes used in other partitions; creating DNA copies from cellular nucleic acids in the edited cells using the barcoded adapter capture primers; creating DNA copies or cDNAs from the editing cassettes in the edited cells using the barcoded cassette capture primers; pooling the nucleic acids from the partitions; sequencing the nucleic acids comprising adapters and the nucleic acids from the editing cassettes; and correlating sequences from the nucleic acids with adapter sequences with the nucleic acids from the editing cassettes.

In some aspects of these embodiments, the barcoded random capture primers comprise random capture primers and first barcoded template switching oligonucleotides, and in some aspects, the barcoded cassette capture primers comprise cassette capture primers and second barcoded template switching oligonucleotides.

In some aspects of these embodiments, the adding step is performed before singulating the edited cells into the partitions and in other aspects, the adding step is performed after singulating the edited cells into the partitions.

In addition to nucleic acid-guided nuclease or nickase fusion editing of cells and correlating the edits to, e.g., resulting cellular nucleic acid profiles, cell surface protein profiles, intracellular protein profiles, transcriptomes, DNA methylation profiles or chromatin accessibility profiles, nucleic acid-guided nuclease or nickase fusion editing may be correlated to combinations of these profiles; that is, one can perform nucleic acid-guided nuclease or nickase fusion editing in a population of cells, then correlate edits with, e.g., both cellular nucleic acid profiles and cell surface protein profiles or both transcriptome and chromatin accessibility.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is a simple process diagram for performing nucleic acid-guided nuclease or nickase fusion editing in a population of cells and determining the resulting cellular nucleic acid profile resulting from one or more edits in individual cells in the population using barcoded cassette capture primers to capture editing cassettes and barcoded random capture primers to capture nucleic acids from each cell. FIG. 1B is a simplified depiction of the process of FIG. 1A.

FIG. 2A is a simple process diagram for performing nucleic acid-guided nuclease or nickase fusion editing in a population of cells and determining the resulting cell surface protein profile resulting from one or more edits in individual cells in the population using barcoded cassette capture primers to capture editing cassettes and barcoded analyte capture primers to capture nucleic acid tags conjugated to antibodies against cell surface proteins in each cell. FIG. 2B is a simplified depiction of the process of FIG. 2A.

FIG. 3A is a simple process diagram for performing nucleic acid-guided nuclease or nickase fusion editing in a population of cells and determining the resulting intracellular protein profile resulting from one or more edits in individual cells in the population using barcoded cassette capture primers to capture editing cassettes and barcoded analyte capture primers to capture nucleic acid tags conjugated to antibodies against intracellular proteins in each cell. FIG. 3B is a simplified depiction of the process of FIG. 3A.

FIG. 4A is a simple process diagram for performing nucleic acid-guided nuclease or nickase fusion editing in a population of cells and determining the resulting genomic DNA methylation profile resulting from one or more edits in individual cells in the population using barcoded cassette capture primers to capture editing cassettes and barcoded random capture primers to capture nucleic acids from each cell. FIG. 4B is a simplified depiction of the process of FIG. 4A. FIG. 4C depicts the process of bisulfite conversion of unmethylated cytosine residues to uracil residues leaving methylated cytosine residues unaffected.

FIG. 5A is a simple process diagram for nucleic acid-guided nuclease or nickase fusion editing in a population of cells and using MNase to determine the chromatin accessibility profile resulting from one or more cellular edits in individual cells in the population using barcoded cassette capture primers to capture editing cassettes and barcoded random capture molecules to capture nucleic acids from each cell. FIG. 5B is a simple process diagram for nucleic acid-guided nuclease or nickase fusion editing in a population of cells and using TN5 transposase to determine the chromatin accessibility profile resulting from one or more cellular edits in individual cells in the population using barcoded cassette capture primers to capture editing cassettes and barcoded random capture primers to capture nucleic acids from each cell. FIGS. 5C-1 and 5C-2 together are a simplified depiction of the method of FIG. 5A where an MNase assay is used to query chromatin accessibility. FIG. 5D is a simplified depiction of the method of FIG. 5B where a Tn5 transposon assay is used to query chromatin accessibility.

FIG. 6A is a depiction of the processes of reverse transcription and template switching for cellular nucleic acids in a cell after random nucleic acid primers and barcoded template switching oligonucleotides have been added to the lysate of an individual cell. FIG. 6B is a depiction of the processes of reverse transcription and template switching for editing cassette transcripts in a cell after cassette capture primers and barcoded template switching oligonucleotides have been added to the lysate of an individual cell. FIG. 6C is a depiction of the process of DNA amplification of the nucleic acids resulting from the cellular nucleic acids and editing cassette extended transcripts.

FIG. 6D is a depiction of size selection of the nucleic acids resulting from the cellular nucleic acid and editing cassette extended transcripts. FIGS. 6E-A and 6E-B together are a depiction of sequencing library generation for the nucleic acids resulting from the cellular nucleic acid and editing cassette extended transcripts where sample indices and P5 and P7 sequencing primer sequences are added to the nucleic acids. FIG. 6F is a depiction of the process of reverse transcription and template switching for mRNA transcripts in a cell after poly-dT primers and barcoded template switching oligonucleotides have been added to the lysate of an individual cell.

FIG. 7A shows the details of the editing cassette captured in the process shown in FIG. 6B, as well as the DNA copy resulting therefrom. FIGS. 7B and 7C detail alternative embodiments of editing cassettes and the DNA copies resulting therefrom.

FIG. 7D details one method for enriching for DNAs derived from the editing cassettes of FIG. 7A using secondary PCR. FIG. 7E details one method for enriching for DNAs derived from the editing cassettes of FIG. 7B using hybrid capture. FIG. 7F details one method for enriching for DNAs derived from the editing cassettes of FIG. 7C using hybrid capture. FIG. 7G details one method for enriching for DNAs derived from the editing cassettes of FIG. 7A using hybrid capture.

FIG. 8A is a simplified diagram of one embodiment of a module that may be employed to perform methods for correlating edits and cellular nucleic acid profiles in a population of cells. FIG. 8B is a simplified diagram of an alternative embodiment of a module that may be employed to perform methods for correlating edits and cellular nucleic acid profiles in a population of cells.

FIGS. 9A-9C depict an embodiment of a solid wall isolation incubation and normalization (SWIIN) module. FIG. 9D depicts the embodiment of the SWIIN module in FIGS. 9A-9C further comprising a heater and a heated cover.

FIG. 10 is a bar graph showing the edit fraction obtained for cells edited and analyzed for phenotypic changes as the result of editing.

It should be understood that the drawings are not necessarily to scale, and that like reference numbers refer to like features.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Viral Vectors (Kaplift & Loewy, eds., Academic Press 1995); all of which are herein incorporated in their entirety by reference for all purposes. For mammalian/stem cell culture and methods see, e.g., Basic Cell Culture Protocols, Fourth Ed. (Helgason & Miller, eds., Humana Press 2005); Culture of Animal Cells, Seventh Ed. (Freshney, ed., Humana Press 2016); Microfluidic Cell Culture, Second Ed. (Borenstein, Vandon, Tao & Charest, eds., Elsevier Press 2018); Human Cell Culture (Hughes, ed., Humana Press 2011); Cell and Tissue Culture: Laboratory Procedures in Biotechnology (Doyle & Griffiths, eds., John Wiley & Sons 1998); Essential Stem Cell Methods, (Lanza & Klimanskaya, eds., Academic Press 2011); and Essentials of Stem Cell Biology, Third Ed., (Lanza & Atala, eds., Academic Press 2013. For background on microfluidic-based emulsion formation and reactions, see Niu and deMello, “Building droplet-based microfluidic systems for biological analysis”, Biochem Soc. Trans., 40:615-623 (2012); Solvas and deMello, “Droplet microfluidics: recent developments and future applications”, Chem. Commun., 47:1936-42 (2011); and Macosko, et al., “Highly Parallel Genome-wide Expression Profiling of Individual Cells Using Nanoliter Droplets”, Cell, 161:1202-14 (2015). CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

An “adapter” or “adapter sequence” is a polynucleotide or oligonucleotide which can be ligated to a nucleic acid.

As used herein, the terms “amplify” or “amplification” and their derivatives, refer to any operation or process whereby at least a portion of a nucleic acid molecule is replicated or copied into at least one additional nucleic acid molecule. The additional nucleic acid molecule may include a sequence that is substantially identical or substantially complementary to at least a portion of the template nucleic acid molecule. The template nucleic acid molecule can be single-stranded or double-stranded, and the additional nucleic acid molecule can be independently single-stranded or double-stranded. Amplification may include linear or exponential replication of a nucleic acid molecule. In certain embodiments, amplification can be achieved using isothermal conditions; in other embodiments, amplification may include thermocycling. In certain embodiments, the amplification is a multiplex amplification and includes the simultaneous amplification of a plurality of target sequences in a single reaction or process. In certain embodiments, “amplification” includes amplification of at least a portion of DNA and RNA based nucleic acids. The amplification reaction(s) can include any of the amplification processes known to those of ordinary skill in the art. In certain embodiments, the amplification reaction(s) includes methods such as polymerase chain reaction (PCR), ligase chain reaction (LCR), or other methods.

The term “barcoded cassette capture primer” refers to a cassette capture primer that comprises one or more barcodes or a cassette capture primer that does not itself comprise one or more barcodes but is combined with a barcoded template switching oligonucleotide where the combination captures and barcodes an editing cassette transcript. A “cassette capture primer” comprises a cassette capture sequence and a primer sequence.

The term “barcoded product capture primer” refers to a product capture primer that comprises one or more barcodes or a product capture primer that does not itself comprise one or more barcodes but is combined with a barcoded template switching oligonucleotide where the combination captures and barcodes cellular nucleic acids. A “product capture primer” comprises a product capture sequence and a primer sequence. Product capture primers may be random (e.g., random n-mer) capture primers, which capture random cellular nucleic acids; poly d-T capture primers, which capture poly-A mRNAs; analyte capture primers, which capture tags from a cellular analyte tagged with a specific nucleic acid; sequence-specific capture primers, which capture specific nucleic acid sequences; or adapter capture primers, which capture cellular analytes tagged with an adapter. These categories of product capture primers are not necessarily distinct and may overlap; that is, analyte capture primers and adapter capture primers may be and typically are sequence-specific capture primers.

The term “barcode” herein refers to a nucleotide index or tag sequence, the presence of which may be utilized to indicate an editing event. A barcode can be any number of nucleotides in length, e.g., 1, 2, 3, 4, 5, 6, 7, 8, 9, 10, 11, 12, 13, 14, 15, 16, 17, 18, 19, 20, or more nucleotides.

The term “cellular nucleic acid profile” refers to the nucleic acids present in the cell, whether genomic DNA, episomic DNA, RNA, or nucleic acid tags used to tag antibodies or other ligands.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term “capture sequence” herein refers to a nucleotide sequence that hybridizes to a nucleotide sequence of interest. The capture sequence may be, in the context of capturing mRNA sequences, poly-dT; in the context of copying cellular nucleic acids generally, random (e.g., random n-mer) hybridization sequences; and in the context of copying sequence-specific nucleic acid tags or adapters, sequences complementary to the sequence-specific nucleic acid tags or adapters or to other sequences inserted into or ligated to cellular nucleic acids. Alternatively, the capture sequence may be a sequence of nucleotides the complement of which is linked to a nucleotide sequence of interest such as, in the context of this disclosure, an editing cassette; that is, the capture sequence may be a specific capture sequence included on all editing cassettes to allow capture.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The term “editing cassette” refers to a nucleic acid molecule comprising a coding sequence for transcription of a guide nucleic acid or gRNA covalently linked to a coding sequence for transcription of a repair template.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease or nickase fusion. In the context of this disclosure, a gRNA is linked to a capture sequence to allow for capture of the gRNA.

“Homology” or “identity” or “similarity” refers to sequence similarity between two peptides or, more often in the context of the present disclosure, between two nucleic acid molecules. The term “homologous region” refers to a region on the repair template with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, the term “nickase fusion” refers to a nucleic acid-guided nickase-(or nucleic acid-guided nuclease or CRISPR nuclease) that has been engineered to act as a nickase rather than a nuclease (e.g., the nickase portion of the fusion functions as a nickase as opposed to a nuclease that initiates double-stranded DNA breaks), where the nickase is fused to a reverse transcriptase, which is an enzyme used to generate cDNA from an RNA template. For information regarding nickase-RT fusions see, e.g., U.S. Pat. No. 10,689,669 and U.S. Ser. No. 16/740,421.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM mutation” refers to one or more edits to a target sequence that removes, mutates, or otherwise renders inactive a PAM or spacer region in the target sequence.

As used herein, the terms “protein” and “polypeptide” are used interchangeably. Proteins may or may not be made up entirely of amino acids.

A “primer” or “primer sequence” is a nucleic acid sequence that can hybridize to a target sequence and function as a starting point for nucleic acid synthesis. Generally, primers act as substrates onto which nucleotides can be polymerized by a polymerase or to which a nucleic acid sequence, e.g., a barcode, can be ligated. In certain examples, the primer itself may be incorporated into the synthesized nucleic acid sequence. In certain examples, the primer is a single stranded polynucleotide or oligonucleotide.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible.

As used herein the term “repair template” refers to nucleic acid that is designed to introduce a DNA sequence modification (insertion, deletion, substitution) into a locus by homologous recombination using nucleic acid-guided nucleases or a nucleic acid that serves as a template (including a desired edit) to be incorporated into target DNA by reverse transcriptase in a nickase fusion editing system.

As used herein the term “selectable marker” or “selection marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The term “specifically binds” as used herein includes an interaction between two molecules, e.g., a capture sequence (e.g., a poly-dT sequence, a random hybridization sequence, tag capture sequence or cassette capture sequence) and the sequence to be captured, with a binding affinity represented by a dissociation constant of about 10⁻⁷ M, about 10⁻⁸ M, about 10⁻⁹ M, about 10⁻¹⁰ M, about 10⁻¹¹ M, about 10⁻¹² M, about 10⁻¹ M, about 10⁻¹⁴ M or about 10⁻¹⁵ M.

The terms “target genomic DNA sequence”, “cellular target sequence”, “target sequence”, or “genomic target locus” refer to any locus in vitro or in vivo, or in a nucleic acid (e.g., genome or episome) of a cell or population of cells, in which a change of at least one nucleotide is desired using a nucleic acid-guided nuclease or nickase fusion editing system. The target sequence can be a genomic locus or extrachromosomal locus.

The terms “transformation”, “transfection” and “transduction” are used interchangeably herein to refer to the process of introducing exogenous DNA into cells.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. In some embodiments, two vectors—an engine vector, comprising the coding sequence for a nuclease or nickase fusion, and an editing vector, comprising the gRNA and the repair template sequence are used. In alternative embodiments, all editing components, including the nuclease or nickase fusion, gRNA, and repair template sequence are all on the same vector (e.g., a combined editing/engine vector).

### Nuclease-Directed or Nickase-Fusion-Directed Genome Editing Generally

The compositions and methods described herein are employed to perform nuclease-directed or nickase-fusion-directed genome editing to introduce desired edits (i.e., intended edits) to a population of cells and then further to correlate the edit made to the resulting cellular nucleic acid profile in individual cells in the population. In some embodiments, recursive cell editing is performed where edits are introduced in successive rounds of editing and the resulting cellular nucleic acid profile can be determined and correlated to the two to many editing cassettes used to make the desired edits. In some embodiments, cellular nucleic acids serve as a proxy for, e.g., cell surface proteins (using, e.g., nucleic acid-tagged antibodies or other ligands), intracellular proteins (also using, e.g., nucleic acid-tagged antibodies or other ligands), and chromatin accessibility.

In the nucleic acid-guided nuclease or nickase fusion editing process, a nucleic acid-guided nuclease or nickase fusion complexed with an appropriate synthetic guide nucleic acid (e.g., gRNA) in a cell cuts or nicks the genome of the cell at a desired location. The guide nucleic acid helps the nucleic acid-guided nuclease or nickase fusion recognize and cut the DNA at a specific target sequence. By manipulating the nucleotide sequence of the guide nucleic acid, the nucleic acid-guided nuclease or nickase fusion may be programmed to target any DNA sequence for cleavage as long as an appropriate protospacer adjacent motif (PAM) is nearby. Thus, the gRNA comprises homology to the target sequence and can be used to track the edit made to the target sequence. In certain aspects, the nucleic acid-guided nuclease or nickase fusion editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease or nickase fusion.

In general, a guide nucleic acid (e.g., gRNA) complexes with a compatible nucleic acid-guided nuclease or nickase fusion and can then hybridize with a target sequence, thereby directing the nuclease or nickase fusion to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may and preferably does reside within an editing cassette. Methods and compositions for designing and synthesizing editing cassettes are described in U.S. Pat. Nos. 10,240,167; 10,266,849; 9,982,278; 10,351,877; 10,364,442; 10,435,715; 10,669,559; 10,711,284; 10,731,180, all of which are incorporated by reference herein in their entirety. Editing cassettes, in addition to paired gRNAs and repair templates, may and typically do in the context of the present disclosure comprise additional sequences such as cassette barcodes and/or cassette capture sequences where the capture sequences facilitate capture of the editing cassettes when analyzing the edit/cellular nucleic acid profile relationship.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease or nickase fusion to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99% or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In general, to generate an edit in the target sequence, the gRNA/nuclease or nickase fusion complex binds to a target sequence as determined by the guide RNA, and the nuclease or nickase fusion recognizes a protospacer adjacent motif (PAM) sequence adjacent to the target sequence. The target sequence can be any polynucleotide endogenous or exogenous to the cell, or in vitro. For example, the target sequence can be a polynucleotide residing in the nucleus of the cell. A target sequence can be a sequence encoding a gene product (e.g., a protein) or a non-coding sequence (e.g., a regulatory polynucleotide, an intron, a PAM, a control sequence, or “junk” DNA).

The guide nucleic acid may be and preferably is part of an editing cassette that encodes the repair template that targets a cellular target sequence. Alternatively, the guide nucleic acid may not be part of the editing cassette and instead may be encoded on the editing vector backbone. For example, a sequence coding for a guide nucleic acid can be assembled or inserted into a vector backbone first, followed by insertion of the repair template in, e.g., an editing cassette. In other cases, the repair template in, e.g., an editing cassette can be inserted or assembled into a vector backbone first, followed by insertion of the sequence coding for the guide nucleic acid. Preferably, the sequence encoding the guide nucleic acid and the repair template are located together in a rationally-designed editing cassette and are simultaneously inserted or assembled via gap repair into a linear plasmid or vector backbone to create an editing vector.

The target sequence is associated with a proto-spacer mutation (PAM), which is a short nucleotide sequence recognized by the gRNA/nuclease or nickase fusion complex. The precise preferred PAM sequence and length requirements for different nucleic acid-guided nucleases or nickase fusions vary; however, PAMs typically are 2-7 base-pair sequences adjacent or in proximity to the target sequence and, depending on the nuclease or nickase fusion, can be 5′ or 3′ to the target sequence. Engineering of the PAM-interacting domain of a nucleic acid-guided nuclease or nickase fusion may allow for alteration of PAM specificity, improve target site recognition fidelity, decrease target site recognition fidelity, or increase the versatility of a nucleic acid-guided nuclease or nickase fusion.

In most embodiments, the genome editing of a target sequence both introduces a desired/intended DNA change to a target sequence, e.g., the genomic DNA of a cell, and removes, mutates, or renders inactive a proto-spacer mutation (PAM) region in the target sequence (e.g., renders the target site immune to further nuclease or nickase fusion binding). Rendering the PAM at the target sequence inactive precludes additional editing of the cell genome at that target sequence, e.g., upon subsequent exposure to a nucleic acid-guided nuclease or nickase fusion complexed with a synthetic guide nucleic acid in later rounds of editing. Thus, cells having the desired target sequence edit and an altered PAM can be selected for by using a nucleic acid-guided nuclease or nickase fusion complexed with a synthetic guide nucleic acid complementary to the target sequence. Cells that did not undergo the first editing event will be cut rendering a double-stranded DNA break, and thus will not continue to be viable. The cells containing the desired target sequence edit and PAM alteration will not be cut, as these edited cells no longer contain the necessary PAM site and will continue to grow and propagate.

As for the nuclease or nickase fusion component of the nucleic acid-guided nuclease editing system, a polynucleotide sequence encoding the nucleic acid-guided nuclease or nickase fusion can be codon optimized for expression in particular cell types, such as bacterial, yeast, and, here, mammalian cells. The choice of the nucleic acid-guided nuclease or nickase fusion to be employed depends on many factors, such as what type of edit is to be made in the target sequence and whether an appropriate PAM is located close to the desired target sequence. Nucleases of use in the methods described herein include but are not limited to Cas 9, Cas 12/CpfI, MAD2, or MAD7, MAD 2007 or other MADzymes (see U.S. Pat. Nos. 9,982,279; 10,337,028; 10,604,746; 10,665,114; 10,640,754, 10,876,102; 10,883,077; 10,704,033; 10,745,678; 10,724,021; 10,767,169; and 10,870,761 for sequences and other details related to MADzymes). Nickase fusion enzymes typically comprise a CRISPR nucleic acid-guided nuclease engineered to cut one DNA strand in the target DNA rather than making a double-stranded cut, and the nickase portion is fused to a reverse transcriptase. For more information on nickases and nickase fusion editing see U.S. Pat. No. 10,689,669 and U.S. Ser. Nos. 16/740,418; 16/740,420 and 16/740,421, both filed 11 Jan. 2020. Here, a coding sequence for a desired nuclease or nickase fusion is typically on an “engine vector” along with other desired sequences such as a selective marker.

Another component of the nucleic acid-guided nuclease or nickase fusion system is the repair template comprising homology to the target sequence. As described above, the repair template is on the same vector and even in the same editing cassette as the guide nucleic acid and preferably is (but not necessarily is) under the control of the same promoter as the gRNA (that is, a single promoter driving the transcription of both the gRNA and the repair template). The repair template is designed to serve as a template for homologous recombination with a target sequence in the case of nucleic acid-guided editing or to serve as a template for reverse transcriptase to incorporate the desired edit(s) into the target nucleic acid. A repair template polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length. In certain preferred aspects, the repair template can be provided as an oligonucleotide of between 20-300 nucleotides, more preferably between 50-250 nucleotides. The repair template comprises one or more regions that are complementary to a portion of the target sequence. When optimally aligned, the repair template overlaps with (is complementary to) the target sequence by, e.g., about 20, 25, 30, 35, 40, 50, 60, 70, 80, 90 or more nucleotides. The repair template comprises regions complementary to the target sequence typically flanking the desired edit(s).

As described in relation to the gRNA, the repair template is preferably provided as part of a rationally-designed editing cassette, which is inserted into an editing vector (in yeast, preferably a linear vector) where the editing vector may comprise a promoter to drive transcription of the gRNA and the repair template when the editing cassette is inserted into the editing vector. Moreover, there may be more than one, e.g., two, three, four, or more gRNA/repair template rationally-designed editing cassettes inserted into an editing vector; alternatively, a single rationally-designed editing cassette may comprise two to several gRNA/repair template pairs, where each gRNA is under the control of separate different promoters, separate like promoters, or where all gRNAs/repair template pairs are under the control of a single promoter. In some embodiments the promoter driving transcription of the gRNA and the repair template (or driving more than one gRNA/repair template pair) is optionally an inducible promoter.

In addition to the repair template, an editing cassette may comprise one or more primer sites. The primer sites can be used to amplify the editing cassette by using oligonucleotide primers; for example, if the primer sites flank one or more of the other components of the editing cassette. In addition, the editing cassette may comprise and in the context of the present compositions and methods preferably does comprise a barcode. A barcode is a unique DNA sequence that corresponds to the repair template sequence such that the barcode can identify the edit made to the corresponding target sequence. The barcode typically comprises four or more nucleotides and is captured to determine what edit was made in the single cell workflow. In some embodiments, the editing cassettes comprise a collection or library of gRNAs and of repair templates representing, e.g., gene-wide or genome-wide libraries of gRNAs and repair templates. Also—particularly in the present embodiments—the editing cassette may have a “capture sequence” common to all editing cassettes in the library of editing cassettes that allows the cassette capture primer to capture all editing cassette transcripts.

The library of editing cassettes is cloned into vector backbones where, e.g., each different repair template is associated with a different barcode. Also, in preferred embodiments, an editing vector or plasmid encoding components of the nucleic acid-guided nuclease or nickase fusion system further encodes a nucleic acid-guided nuclease or nickase fusion comprising one or more nuclear localization sequences (NLSs), such as about or more than about 1, 2, 3, 4, 5, 6, 7, 8, 9, 10, or more NLSs, particularly as an element of the nuclease or nickase fusion sequence. In some embodiments, the engineered nuclease or nickase fusion comprises NLSs at or near the amino-terminus, NLSs at or near the carboxy-terminus, or a combination.

### Correlating Genome Edits to Changes in Cellular Nucleic Acid and Product Profiles

The present disclosure is drawn to methods, compositions and modules that allow correlation between a genome edit or change and the cellular nucleic acid profile resulting from that change. The present methods allow for multiplex editing in a large population of cells using a library of different editing vectors where each editing vector comprises a different editing cassette. Once the edits have taken place the present methods allow for correlating the resulting edit(s) (via DNA copies or cDNAs of the editing cassettes) to the resulting cellular nucleic acid profile of selected or all nucleic acids in the cell. However, in addition to nucleic acid-guided nuclease or nickase fusion editing of cells and correlating the edits to, e.g., resulting cellular nucleic acid profiles, cell surface protein profiles, intracellular protein profiles, transcriptomes, DNA methylation profiles or chromatin accessibility profiles, nucleic acid-guided nuclease or nickase fusion editing may be correlated to combinations of these profiles; that is, one can perform nucleic acid-guided nuclease or nickase fusion editing in a population of cells, then correlate edits with, e.g., both cellular nucleic acid profiles and cell surface protein profiles or both transcriptome and chromatin accessibility.

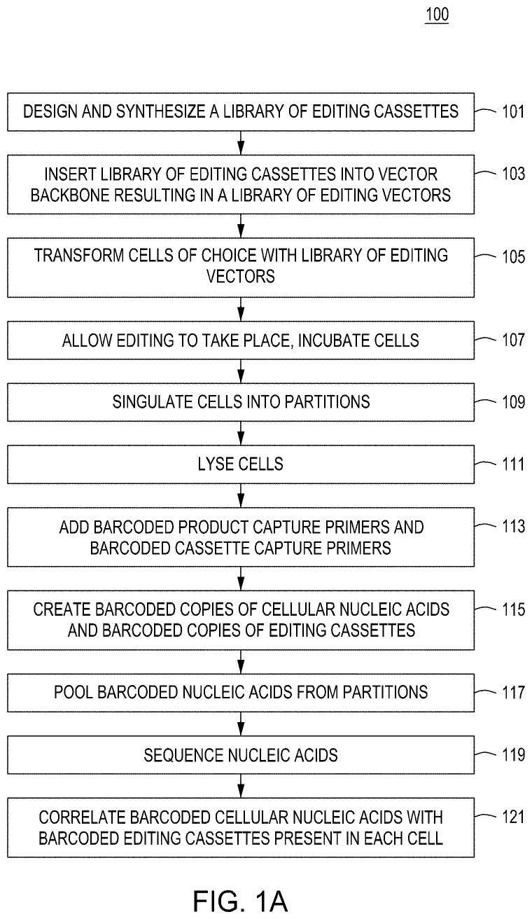

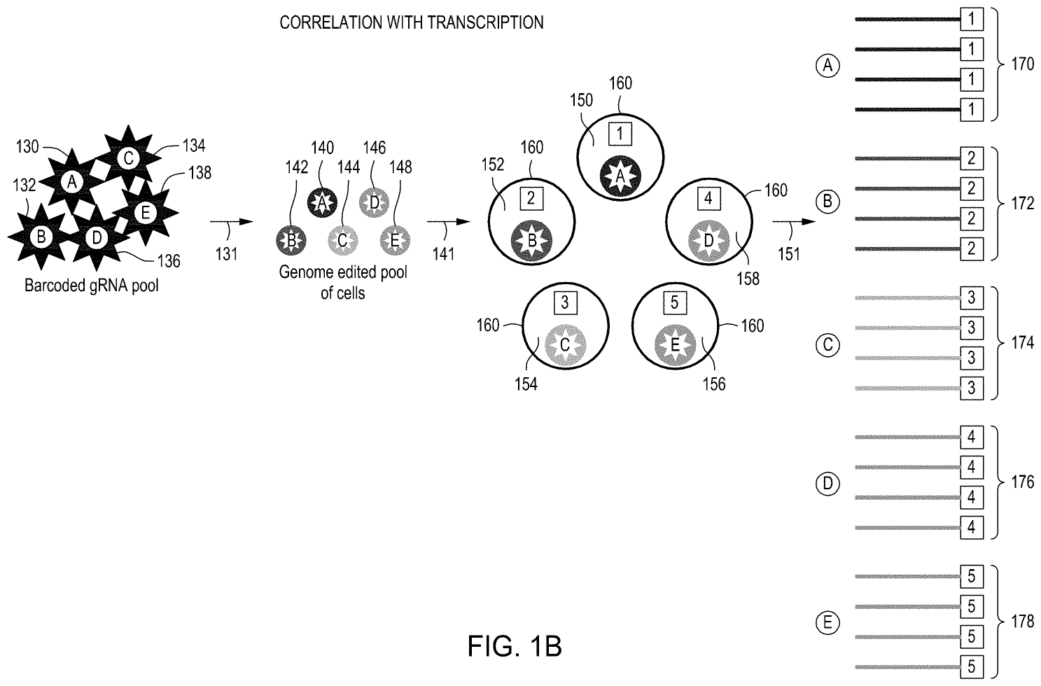

Cellular Nucleic Acid Profile FIG. 1A is a simple process diagram for a method 100 for nucleic acid-guided nuclease or nickase fusion editing in a population of cells and determining the nucleic acid profile resulting from one or more edits in individual cells in the population using barcoded cassette capture primers to capture editing cassettes and barcoded product capture primers—here, random capture primers—to capture nucleic acids from each cell. The barcoded cassette capture primers and barcoded product capture primers facilitate synthesis of DNA copies or cDNAs created from the editing cassettes and cellular nucleic acids.

In a first step 101, a library of editing cassettes comprising paired gRNAs and repair templates is designed and synthesized. In addition to paired gRNAs and repair templates, the editing cassettes typically comprise additional sequences such as one or more priming sequences that can be used to amplify the editing cassette, an editing cassette barcode and/or a capture sequence, where the capture sequence facilitates capture of the editing cassette by the cassette capture primers when analyzing the edit/cellular nucleic acid profile relationship. Once designed and synthesized 101, the library of editing cassettes is amplified, purified and inserted 103 into a vector backbone—which in some embodiments may already comprise a coding sequence for a nuclease or nickase fusion—to produce a library of editing vectors. Alternatively, the coding sequence for a nuclease or nickase fusion may be located on another vector or may be integrated into the cellular genome. In yet another alternative, the nuclease or nickase fusion may be delivered to the cell as a protein. The vectors chosen for the methods herein will vary depending on the type of cells being edited and analyzed, where the vectors include, e.g., plasmids, BACs, YACs, viral vectors and synthetic chromosomes.

The cells of interest useful in the methods herein are any cells, including bacterial, yeast and animal (including mammalian) cells. These cells are provided and are transformed with the library of editing vectors 105. Transformation is intended to generically include a variety of art-recognized techniques for introducing an exogenous nucleic acid sequence (e.g., an engine and/or editing vector) into a target cell, and the term “transformation” as used herein includes all transformation, transfection and transduction techniques. Such methods include, but are not limited to, electroporation, lipofection, optoporation, injection, microprecipitation, microinjection, liposomes, particle bombardment, sonoporation, laser-induced poration, bead transfection, calcium phosphate or calcium chloride co-precipitation, or DEAE-dextran-mediated transfection. Cells can also be prepared for vector uptake using, e.g., a sucrose, sorbitol or glycerol wash. Additionally, hybrid techniques that exploit the capabilities of mechanical and chemical transfection methods can be used, e.g., magnetofection, a transfection methodology that combines chemical transfection with mechanical methods. In another example, cationic lipids may be deployed in combination with gene guns or electroporators. Suitable materials and methods for transforming or transfecting target cells can be found, e.g., in Green and Sambrook, Molecular Cloning: A Laboratory Manual, 4th, ed., Cold Spring Harbor Laboratory Press, Cold Spring Harbor, N.Y., 2014).

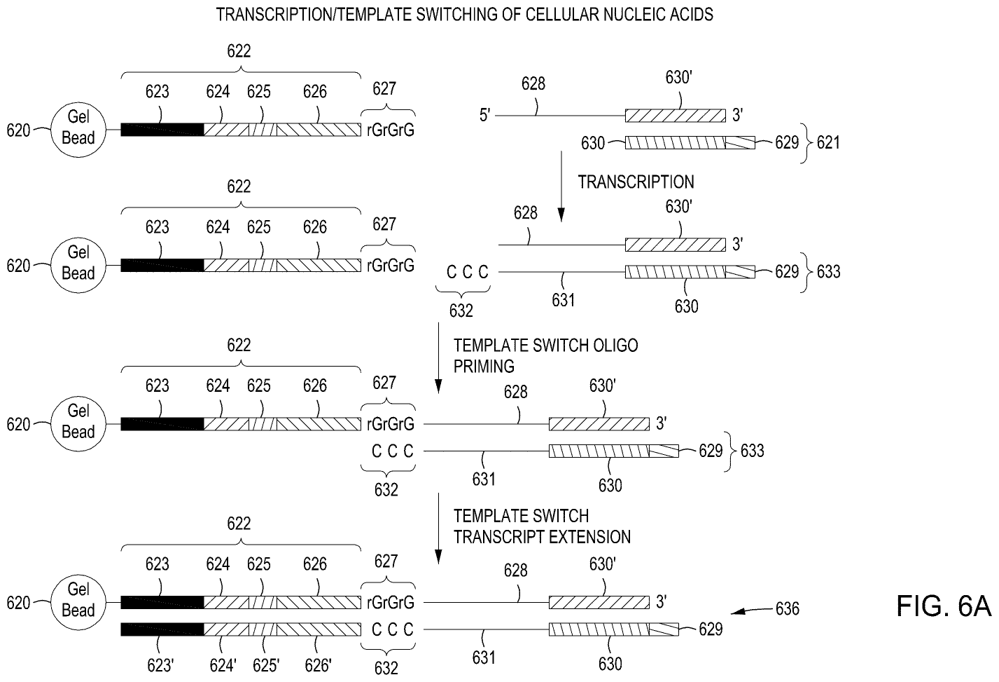

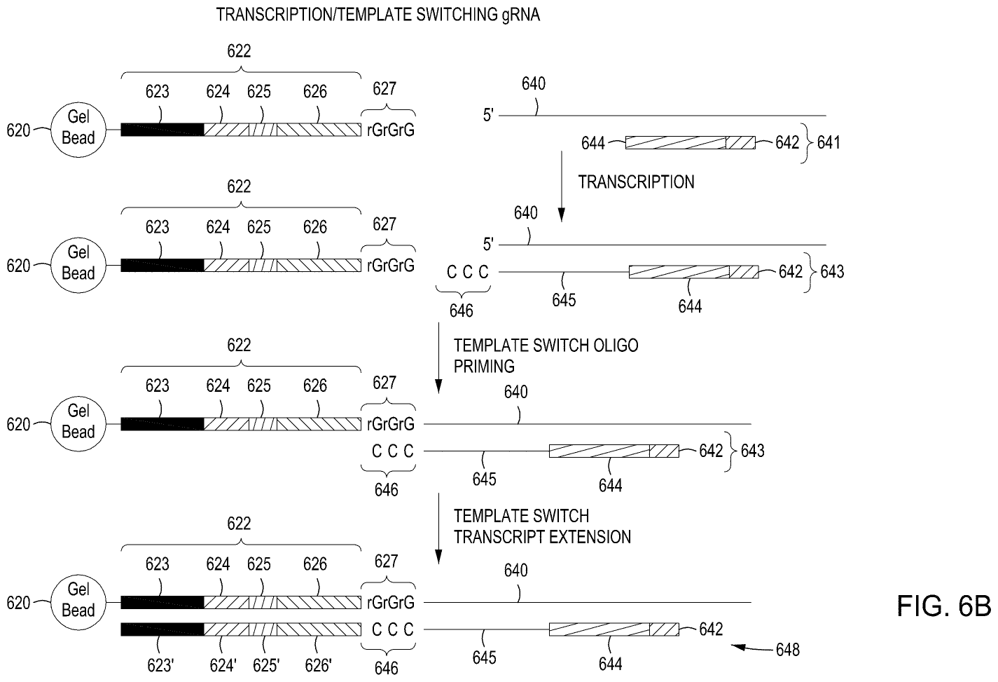

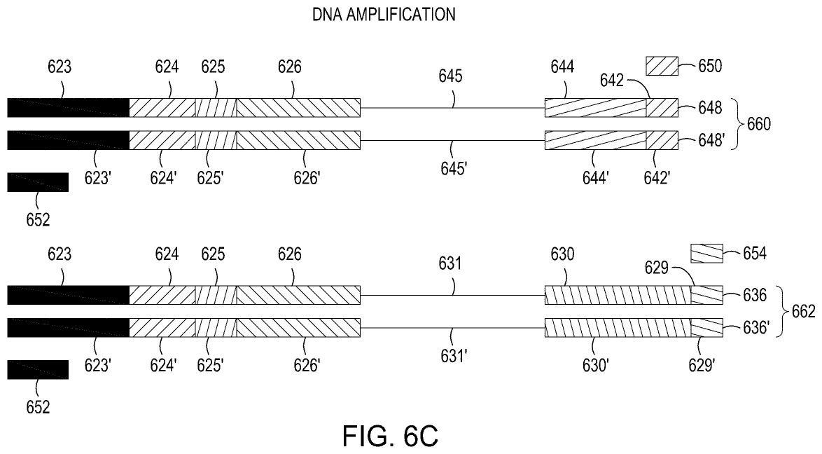

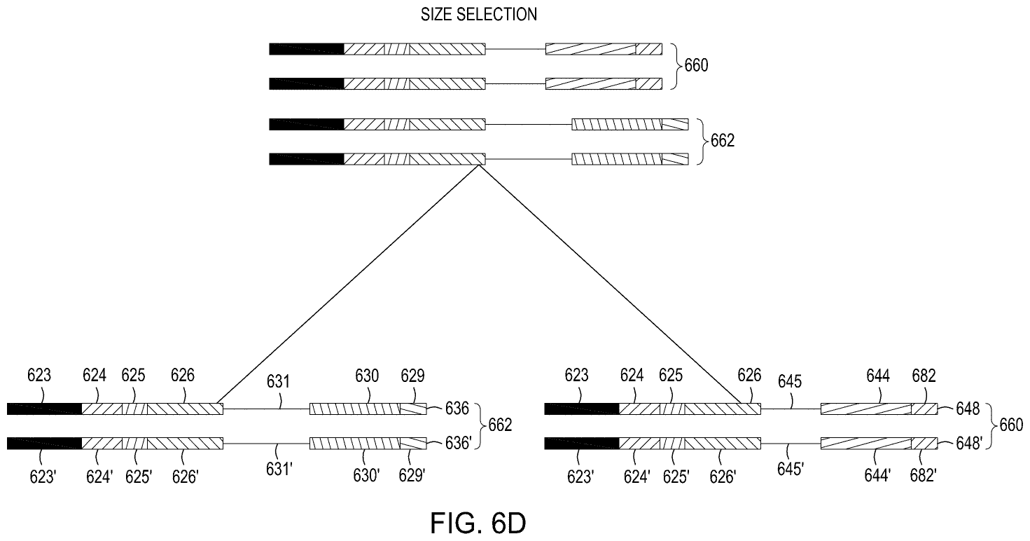

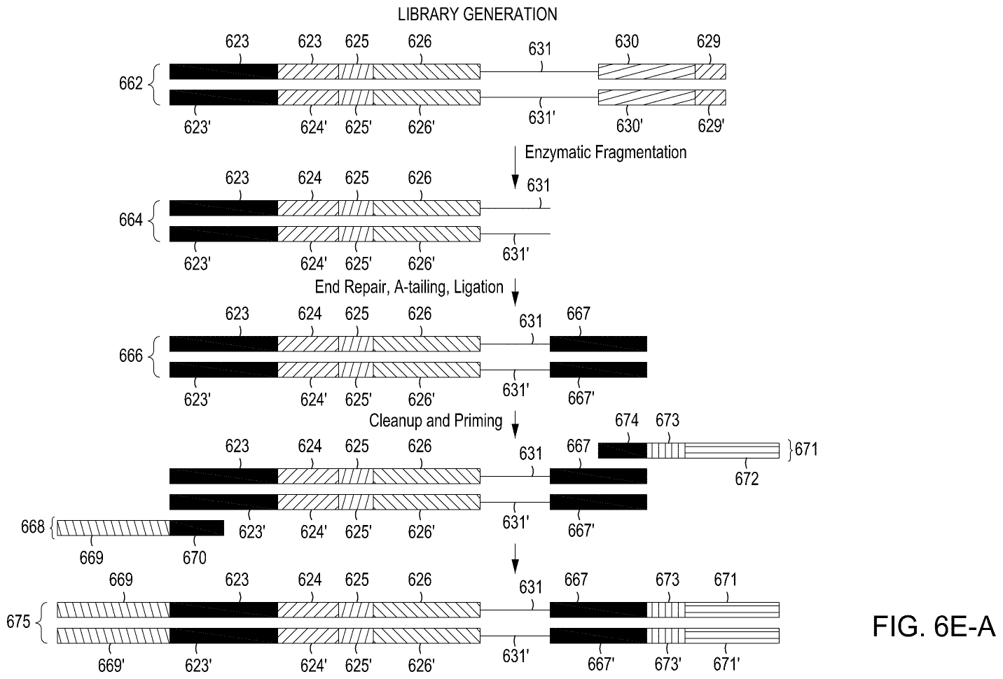

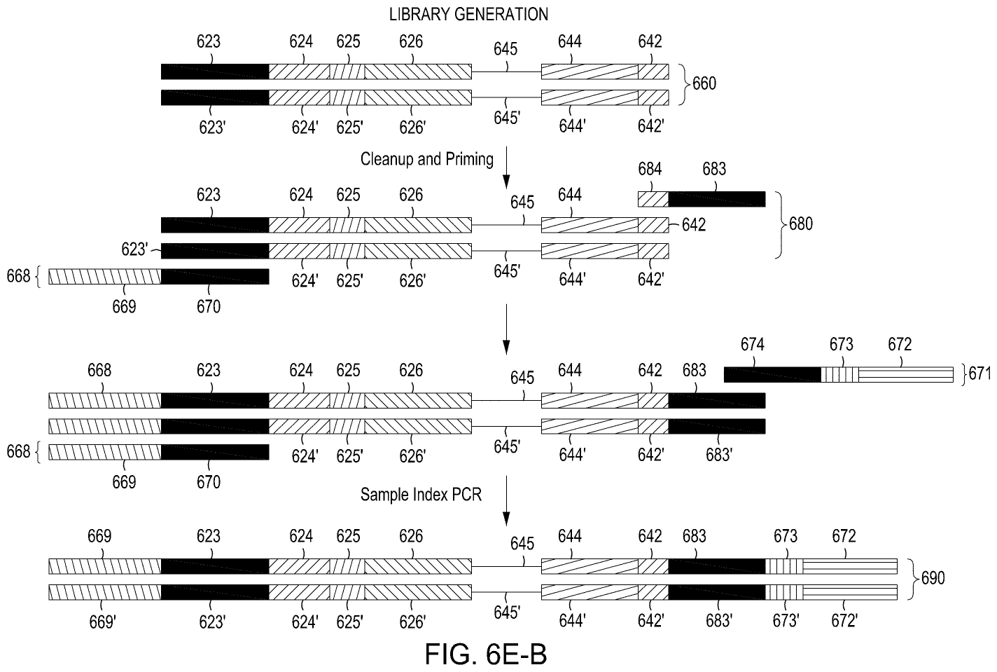

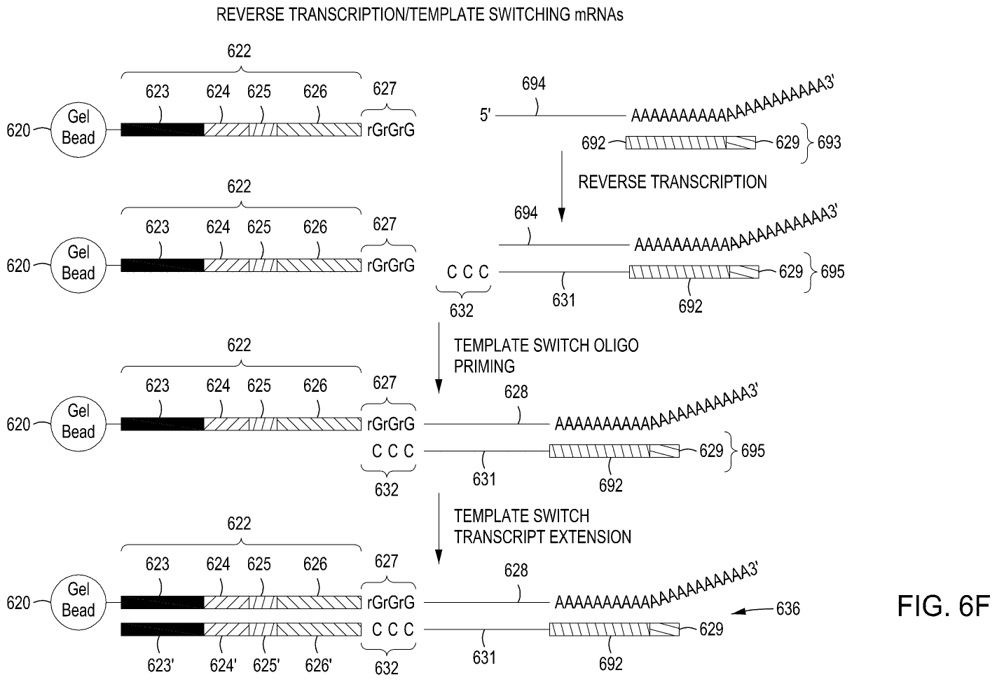

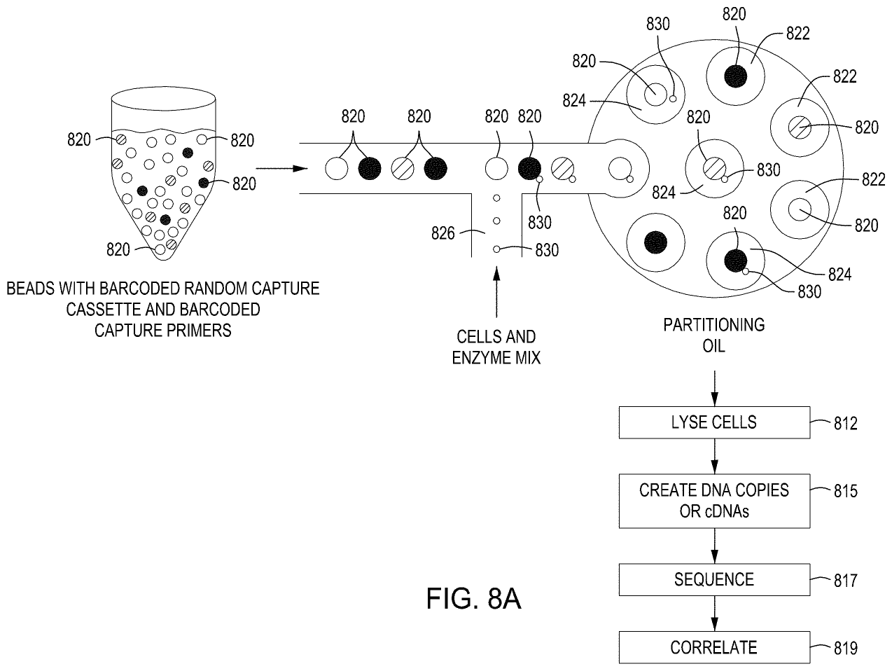

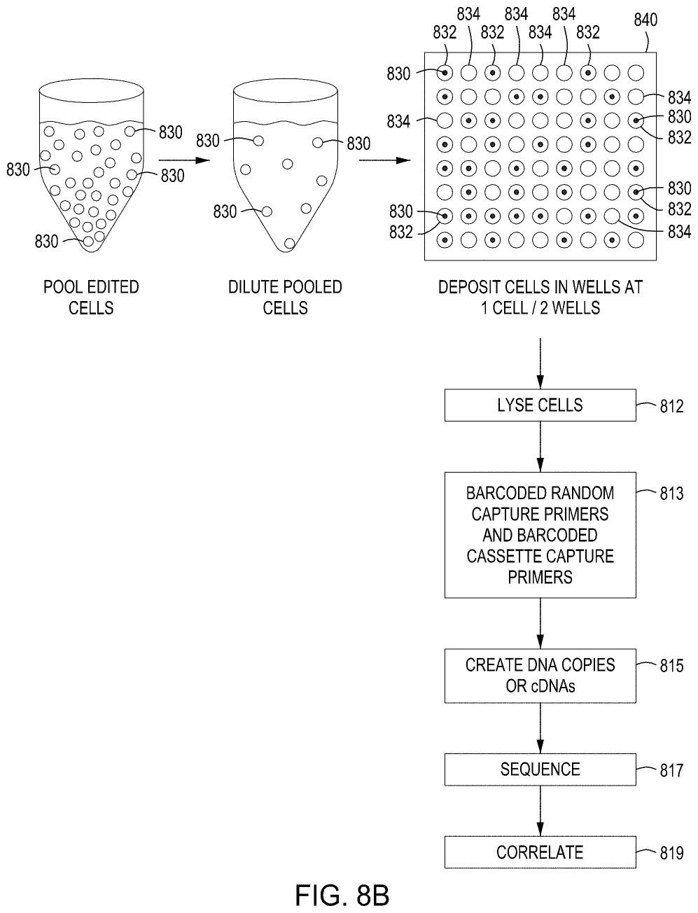

Once transformed 105, the cells are allowed to recover and selection optionally is performed to select for cells transformed with the editing vector, which most often comprises a selectable marker. Drug selectable markers such as gentamicin, bleomycin, streptomycin, puromycin, hygromycin, blasticidin, and G418 or other selectable markers may be employed. At a next step, conditions are provided such that editing takes place and the cells are incubated to allow for transcription and translation of nucleic acids and proteins, respectively, 107 upon which time editing commences. Once editing is complete, the edited cells are singulated into partitions 109 then lysed 111. The partitions may be droplets or gel beads as described in relation to FIG. 8A or the partitions may be wells as described in relation to FIG. 8B, all of which are described in detail infra. Once the cells are lysed to release the nucleic acids present in each cell, the barcoded random capture primers (for example, the random capture primers and barcoded template switching oligonucleotides as described in detail in relation to FIGS. 6A-6F) and barcoded cassette capture primers (for example, the cassette capture primers and barcoded template switching oligonucleotides as described in detail in relation to FIGS. 6A-6F) are added 113. Note that in some implementations the random capture primers, cassette capture primers and barcoded template switching oligonucleotides may be added to the partitions prior to the cells being added to the partitions and lysed, such as shown in FIG. 8A.

The barcoded random capture primers and barcoded cassette capture primers in each partition comprise a different, unique cellular barcode (or one or more unique cellular barcodes) and thus the nucleic acids from the singulated cell in each partition are labeled with this unique cellular barcode. After the barcoded random capture primers and barcoded cassette capture primers are added, DNA copies or cDNAs are created from the cellular nucleic acids and from the editing cassettes present in the cell 115 using a combination of one or more of the processes of priming, transcription or reverse transcription, extension and amplification. After the DNA copies or cDNAs are synthesized, they are pooled 117 and sequenced 119. Because each partition comprised barcoded random capture primers and barcoded cassette capture primers with a unique cellular barcode, each nucleic acid from each cellular nucleic acid and each nucleic acid from each editing cassette transcript from each cell is tagged with this unique cellular barcode; thus, the nucleic acids representing the cellular nucleic acids and the nucleic acids representing the editing cassettes from each partition can be correlated 121.

FIG. 1B is a simplified depiction of the process of FIG. 1A. At left in FIG. 1B is a pool of editing vectors (130, 132, 134, 136, and 138), where each editing vector comprises a different editing cassette (e.g., a gRNA and repair template pair; in FIG. 1B, editing vector 130 comprises editing cassette A, editing vector 132 comprises editing cassette B, editing vector 134 comprises editing cassette C, editing vector 136 comprises editing cassette D, editing vector 138 comprises editing cassette E) and the editing cassette optionally comprises a barcode (or more than one barcode) that uniquely identifies the intended edit to be made by the gRNA and repair template pair. If the editing cassette does not comprise a unique barcode, the editing cassette itself serves as the “barcode.” At step 131 a population of cells is transformed with the pool of editing vectors and conditions are provided to promote nucleic acid-guided nuclease or nickase fusion editing in the cells, producing a genome-edited pool of cells (e.g., cell 140 (blue) edited by editing vector 130, cell 142 (red) edited by editing vector 132, cell 144 (aqua) edited by editing vector 134, cell 146 (green) edited by editing vector 136 and cell 148 (orange) edited by editing vector 138).

At step 141 the cells are singulated into partitions 160 with barcoded random capture primers (here, the barcoded product capture primers are barcoded random capture primers) and barcoded cassette capture primers or random capture primers, cassette capture primers and barcoded template switching oligonucleotides. The cellular barcodes present in a single partition are the same barcode and different partitions have different barcodes. (Note, however, that in some embodiments, the cassette capture primers and random capture primers may be located on the same molecule such that only a single barcode is needed.) Partition 150 comprises cell 140 with barcode 1, partition 152 comprises cell 142 with barcode 2, partition 154 comprises cell 144 with barcode 3, partition 156 comprises cell 146 with barcode 4 and partition 158 comprises cell 148 with barcode 5. At step 151, the cells are lysed, DNA copies or cDNAs are prepared from the cellular nucleic acids and the editing cassettes, the barcoded DNA copies or cDNAs are pooled, and finally the nucleic acids from the cellular nucleic acids are correlated with the editing cassettes present in each cell. The nucleic acids of group 170 (blue) are associated with editing cassette A and cellular barcode 1; the nucleic acids (red) of group 172 are associated with editing cassette B and cellular barcode 2; the nucleic acids (aqua) of group 174 are associated with editing cassette C and cellular barcode 3; the nucleic acids (green) of group 176 are associated with editing cassette D and cellular barcode 4; and the nucleic acids (orange) of group 178 are associated with editing cassette E and cellular barcode 5.

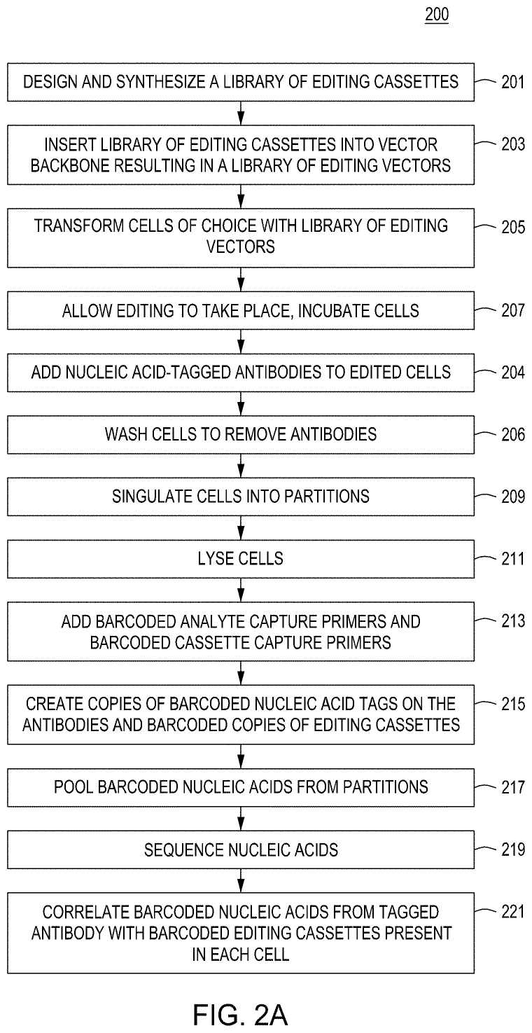

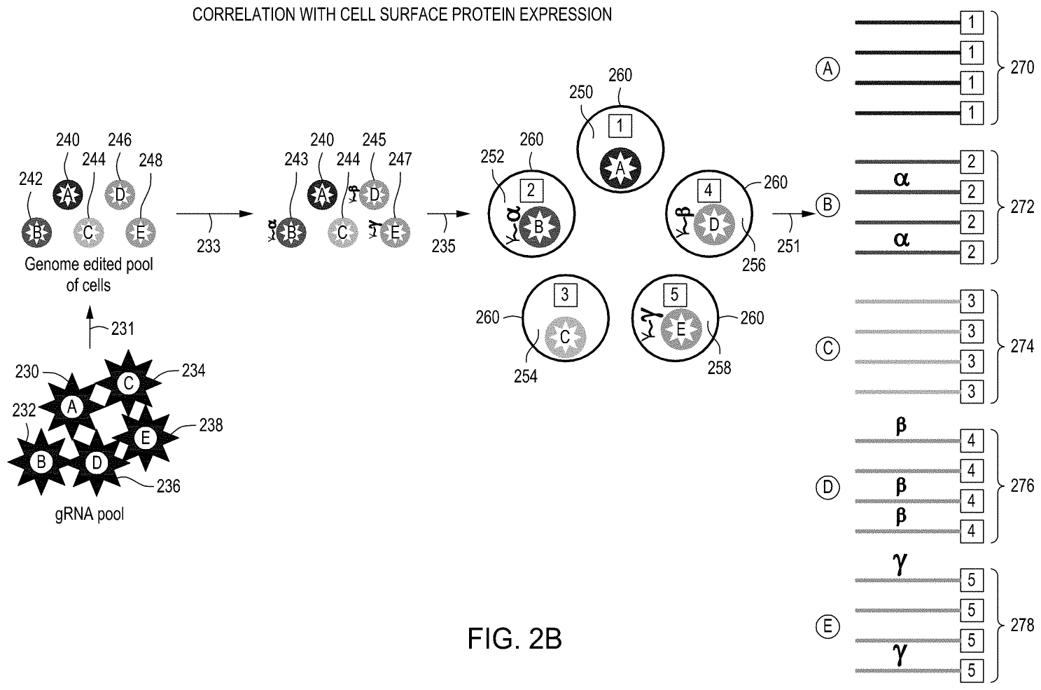

Cell Surface Protein Profile FIG. 2A is a simple process diagram for nucleic acid-guided nuclease or nickase fusion editing of a population of cells and determining the cell surface protein profile resulting from an edit in individual cells in the population using barcoded cassette capture primers to capture editing cassettes and barcoded product capture primers—here, analyte capture primers—to capture nucleic acids from each cell. As described above, in some embodiments, the cassette capture primers and analyte capture primers may themselves comprise a barcode; alternatively, cassette capture primers, analyte capture primers and barcoded template switching oligonucleotides are used (see, e.g., FIGS. 6A-6F). In this embodiment, an analyte capture primer is used to capture cellular nucleic acids—specifically, DNA copies or cDNAs of tags used to tag, e.g., antibodies to cell surface proteins from individual cells. Similar to method 100 described above, method 200 employs various combinations of one or more of the processes of priming, reverse transcription or transcription, extension and amplification. In general, cell surface proteins, also known as cell surface antigens, are special proteins attached to the cell membrane. While some proteins have the task of allowing the transport of molecules across the membrane, cell surface markers also play a role in inter-cellular communication and recognition as well as serving as monograms to help identify and classify cells. In addition to cell surface proteins, lipids and other cell surface molecules can be tagged if there are appropriate antibodies that can identify such cell surface molecules.

Method 200 takes advantage of the diversity of nucleic acid sequences to detect analytes—in this exemplary embodiment, cell surface proteins—since multiplex detection of proteins is more challenging. In method 200, specific oligonucleotides are conjugated to antibodies that detect cell surface proteins. Upon binding of the oligonucleotide-conjugated antibodies to their cognate cell surface proteins, the quantity of the cell surface proteins can be converted to nucleic acid quantity; that is, the presence (and relative quantity) of a nucleic acid tag serves as a proxy for the presence (and relative quantity) of cell surface proteins detected by the antibodies. To date, various methods have been developed to conjugate oligonucleotides to antibodies. For example, heterobifunctional cross-linkers, such as succinimidyl 4-hydrazinonicotinate acetone hydrazine (SANH) and succinimidyl 4-(N-maleimidomethyl) cyclohexane-1-carboxylate (SMCC) are often used to introduce a bridge between the oligonucleotide and the antibody (see, e.g., Moncau, et al., Proteomics, 11:2063-70 (2011) and Soderberg, et al., Nat. Methods, 3:995-1000 (2006), respectively). Commercial kits are also available for the production of oligonucleotide-conjugated antibodies. Examples include the Solulink Antibody-Oligonucleotide All-in-One Conjugation Kit (TriLink Biotechnologies, San Diego, Calif., USA) and the Thunder-Link® PLUS Oligo Conjugation System (Expedeon, Heidelberg, Germany). In some embodiments, conjugation of an oligonucleotide to an antibody or other protein is based on alkyne-azide cycloaddition (the Cu-free click reaction), in which the antibody is activated with a dibenzocyclooctyne moiety and subsequently linked covalently with an azide-modified oligonucleotide (see Manova, et al., Langmuir, 28:8651-63 (2012) and van Hest and van Delft, ChemBioChem, 12:1309-12(2011)).

In a first step 201, a library of editing cassettes comprising paired gRNAs and repair templates is designed and synthesized. In addition to the paired gRNAs and repair templates, the editing cassettes typically comprise additional sequences such as one or more priming sequences, an editing cassette barcode, as well as a capture sequence, where the capture sequence facilitates capture of the editing cassette by the cassette capture primers when analyzing the edit/cellular nucleic acid profile relationship. Once designed and synthesized 201, the library of editing cassettes is amplified, purified and inserted 203 into a vector backbone-which in some embodiments may already comprise a coding sequence for a nuclease or nickase fusion to produce a library of editing vectors. As described in relation to method 100 above, alternatively the coding sequence for the nuclease or nickase fusion may be located on another vector or may be integrated into the cellular genome or the nuclease or nickase fusion may be delivered to the cell as a protein.

As with method 100, the cells of interest useful in method 200 herein include any cells, including bacterial, yeast and animal (including mammalian) cells. These cells are provided and are transformed with the library of editing vectors 205. Again, transformation is intended to generically include a variety of art-recognized techniques for introducing an exogenous nucleic acid sequence (e.g., an engine and/or editing vector) into a target cell, and the term “transformation” as used herein includes all transformation, transfection and transduction techniques. Once transformed 205, the cells are allowed to recover and selection optionally is performed to select for cells transformed with the editing vector, which most often comprises a selectable marker. As described supra, drug selectable markers such as gentamicin, bleomycin, streptomycin, puromycin, hygromycin, blasticidin, and G418 or other selectable markers may be employed. At a next step, conditions are provided such that editing takes place where the cells are incubated to allow for transcription and translation of nucleic acids and proteins, respectively 207.

Once editing is complete and the cells have produced nucleic acids and proteins (including those resulting from the intended edits), nucleic acid-tagged probes—here, nucleic acid-tagged antibodies to cell surface proteins, where antibodies against different cell surface proteins are tagged with different nucleic acid tags—are added to the population of edited cells in bulk and the cells are incubated to allow the tagged probes to bind to cell surface proteins on the surface of the population of cells 204. In the present exemplary embodiment, the cell surface analyte is a cell surface protein; however, it should be understood by one of ordinary skill in the art given the present disclosure that the cell surface analyte may be any analyte for which there is a specific probe. Similarly, the analyte probe in the present embodiment is an antibody; however, the probe may be an aptamer or other ligand that binds specifically to a cell surface analyte and can be conjugated to a nucleic acid tag or mixtures thereof.

Following incubation with the tagged probes (e.g., nucleic acid-tagged antibodies), the cells are washed to remove unbound antibodies 206, and the edited cells are singulated into partitions 209 then lysed 211. Again, the partitions may be droplets or gel beads as described in relation to FIG. 8A or the partitions may be wells as described in relation to FIG. 8B, both of which are described in detail infra. Once the cells are lysed to release the nucleic acids present in each cell, barcoded analyte capture primers (e.g., analyte capture primers and barcoded template switching oligonucleotides as described in detail in relation to FIGS. 6A-6F) and barcoded cassette capture primers (e.g., the cassette capture primers and barcoded template switching oligonucleotides as described in detail in relation to FIGS. 6A-6F) are added 213. Note—as described supra—in some implementations the barcoded analyte capture primers and barcoded cassette capture primers may be added to the partitions prior to the cells being added to the partitions and lysed, such as shown in FIG. 8A. Note that some exemplary embodiments use barcoded template switching oligonucleotides for barcoding the nucleic acid tags conjugated to the antibodies or barcoding the editing cassettes; however, in other embodiments, the analyte tag capture primers and cassette capture primers may themselves be barcoded.

The barcoded analyte capture primers and barcoded cassette capture primers in each partition comprise a unique cellular barcode (or one or more cellular barcodes) and thus the nucleic acids from the singulated cell in each partition is labeled with this unique cellular barcode. After the barcoded analyte capture primers and barcoded cassette capture primers are added, DNA copies or cDNAs are created from the nucleic acid tags on the antibodies that bound to the cell surface proteins and from the editing cassettes present in the cell 215 using a combination of one or more of the processes of priming, transcription or reverse transcription, extension and amplification. After the DNA copies or cDNAs are synthesized, they are pooled 217 and sequenced 219. Because each partition comprised barcoded analyte capture primers and barcoded cassette capture primers with a unique cellular barcode, each nucleic acid copy from each nucleic acid tag and each nucleic acid copy from each editing cassette transcript from each cell is tagged with this unique cellular barcode; thus, the nucleic acids representing the nucleic acid tags and the nucleic acids representing the editing cassettes from each partition can be correlated 221.

FIG. 2B is a simplified depiction of the process of FIG. 2A. At left in FIG. 2B is a pool of editing vectors (230, 232, 234, 236, and 238), where each editing vector comprises a different editing cassette (e.g., a gRNA and repair template pair; in FIG. 2B, editing vector 230 comprises editing cassette A, editing vector 232 comprises editing cassette B, editing vector 234 comprises editing cassette C, editing vector 236 comprises editing cassette D, editing vector 238 comprises editing cassette E) and the editing cassette optionally comprises a barcode (or more than one barcode) that uniquely identifies the intended edit to be made by the gRNA and repair template pair. If the editing cassette does not comprise a unique barcode, the editing cassette itself serves as the “barcode.” At step 231 a population of cells is transformed with the pool of editing vectors and the conditions are provided to promote nucleic acid-guided nuclease or nickase fusion editing in the cells, producing a genome-edited pool of cells (e.g., cell 240 (blue) edited by editing vector 230; cell 242 (red) edited by editing vector 232; cell 244 (aqua) edited by editing vector 234; cell 246 (green) edited by editing vector 236; and cell 248 (orange) edited by editing vector 238). At step 233, nucleic acid-tagged antibodies are added to the edited cells and allowed to bind to cell surface proteins on the cells. Here, three different antibodies each with its own nucleic acid tag are added, where the different antibodies are labeled a (bound to the surface of cell 243), R (bound to the surface of cell 245) and 7 (bound to the surface of cell 247).

At step 235, the cells are washed to remove unbound antibodies, singulated into partitions 260 with barcoded analyte capture primers and barcoded cassette capture primers or analyte capture primers, cassette capture primers and barcoded template switching oligonucleotides. As described in relation to FIG. 1B, cellular barcodes present in a single partition are the same cellular barcode and different partitions have different cellular barcodes. (Note, however, that in some embodiments, the cassette capture primers and random capture primers may be located on the same molecule such that only a single barcode is necessary.) Partition 250 comprises cell 240 with barcode 1; partition 252 comprises cell 243 (tagged by the a antibody) with barcode 2; partition 254 comprises cell 244 with barcode 3, partition 256 comprises cell 245 (tagged by the R antibody) with barcode 4 and partition 258 comprises cell 247 (tagged by the 7 antibody) with barcode 5. At step 251, the cells are lysed, DNA copies or cDNAs are prepared of the nucleic acid tags and the editing cassettes, the barcoded DNA copies or cDNAs are pooled, and finally the copies of the nucleic acid tags are correlated with the editing cassettes present in each cell. The DNA copies or cDNAs of group 270 (blue) are associated with editing cassette A and cellular barcode 1; the DNA copies or cDNAs of group 272 (red) are associated with editing cassette B, cellular barcode 2 and the a nucleic acid-tagged antibody (there are 2 tagged sequences); the DNA copies or cDNAs of group 274 (aqua) are associated with editing cassette C and cellular barcode 3; the DNA copies or cDNAs of group 276 (green) are associated with editing cassette D, cellular barcode 4 and the R nucleic acid-tagged antibody (there are 3 tagged sequences); and the DNA copies or cDNAs of group 278 (orange) are associated with editing cassette E, cellular barcode 5 and the 7 nucleic acid-tagged antibody (there are 2 tagged sequences).

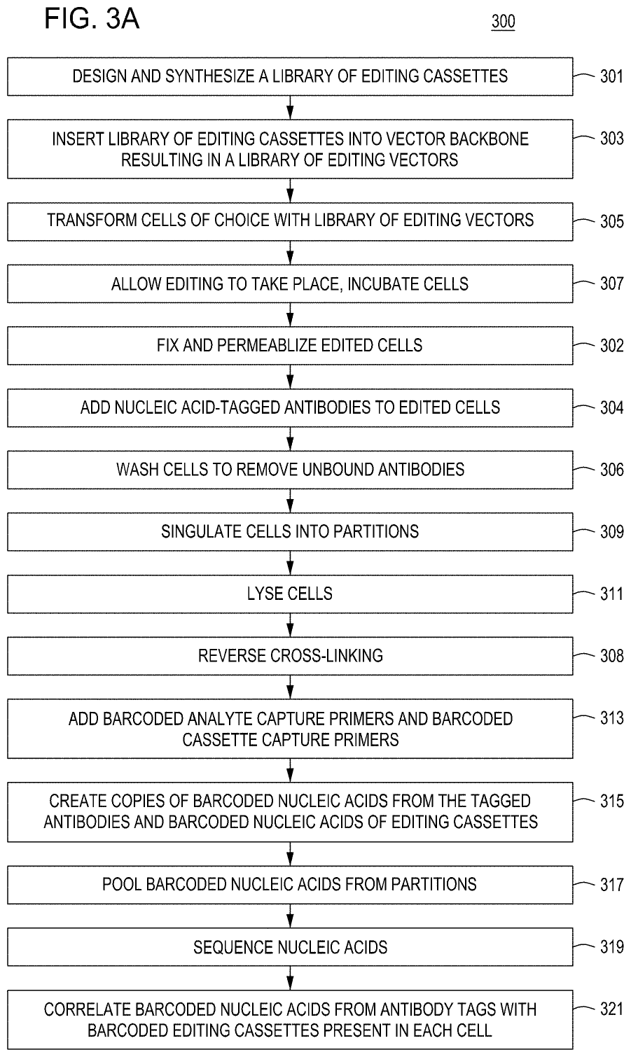

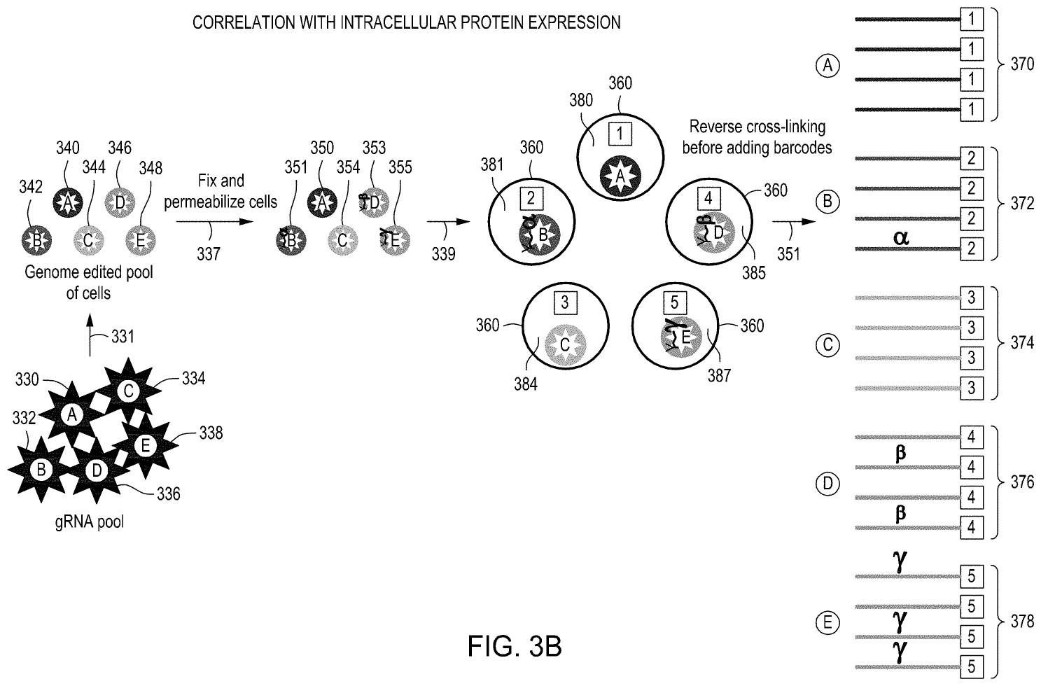

Intracellular Protein Profile FIG. 3A is similar to FIG. 2A as it also addresses detecting and quantifying cellular proteins. FIG. 3A a simple process diagram for nucleic acid-guided nuclease or nickase fusion editing in a population of cells and determining the intracellular protein profile resulting from one or more edits in individual cells in the population using barcoded cassette capture primers to capture editing cassettes and barcoded product capture primers—here, barcoded analyte capture primers—to capture nucleic acid tags conjugated to antibodies from each cell. As described herein, in some embodiments, cassette capture primers, analyte capture primers, and barcoded template switching oligonucleotides are used (see, e.g., FIGS. 6A-6F); alternatively the cassette capture primers and analyte capture primers themselves may carry a barcode or the cassette capture primers and analyte capture primers may be located on a single molecule, in which case a single barcode would be sufficient. In this embodiment, an analyte capture primer is used to capture cellular nucleic acids-specifically, DNA copies or cDNAs of tags used to tag, e.g., antibodies to intracellular proteins from individual cells. Like method 200, method 300 employs various combinations of one or more of the processes of priming, transcription or reverse transcription, extension and amplification.

The embodiment of method 300, like method 200, takes advantage of the diversity of nucleic acid sequences to detect analytes—in this exemplary embodiment, intracellular proteins—since multiplex detection of proteins is more challenging. In method 300, specific oligonucleotides are conjugated to antibodies that bind to intracellular proteins. Upon binding of the oligonucleotide-conjugated antibodies to their target intracellular proteins, the quantity of the intracellular proteins can be converted to nucleic acid quantity; that is, the presence (and quantity) of a nucleic acid tag serves as a proxy for the presence (and quantity) of intracellular proteins detected by the antibodies. As described supra in relation to FIG. 2A, to date various methods have been developed to conjugate oligonucleotides to antibodies. For example, heterobifunctional cross-linkers, such as succinimidyl 4-hydrazinonicotinate acetone hydrazine (SANH) and succinimidyl 4-(N-maleimidomethyl) cyclohexane-1-carboxylate (SMCC) are often used to introduce a bridge between the oligonucleotide and the antibody (see, e.g., Moncau, et al., Proteomics, 11:2063-70 (2011) and Soderberg, et al., Nat. Methods, 3:995-1000 (2006), respectively). Commercial kits are also available for the production of oligonucleotide-conjugated antibodies with examples including the Solulink Antibody-Oligonucleotide All-in-One Conjugation Kit (TriLink Biotechnologies, San Diego, Calif., USA) and the Thunder-Link® PLUS Oligo Conjugation System (Expedeon, Heidelberg, Germany). In some embodiments, conjugation of an oligonucleotide to an antibody or other protein is based on alkyne-azide cycloaddition (the Cu-free click reaction), in which the antibody is activated with a dibenzocyclooctyne moiety and subsequently linked covalently with an azide-modified oligonucleotide (see Manova, et al., Langmuir, 28:8651-63 (2012) and van Hest and van Delft, ChemBioChem, 12:1309-12 (2011)).

In a first step 301, a library of editing cassettes comprising paired gRNAs and repair templates is designed and synthesized. In addition to the paired gRNAs and repair templates, the editing cassettes typically comprise additional sequences such as one or more priming sequences, an editing cassette barcode, as well as a capture sequence, where the capture sequence facilitates capture of the editing cassette by the cassette capture primers when analyzing the edit/cellular nucleic acid profile relationship. Once designed and synthesized 301, the library of editing cassettes is amplified, purified and inserted 303 into a vector backbone—which in some embodiments may already comprise a coding sequence for a nuclease or nickase fusion—to produce a library of editing vectors. As described in relation to method 200 above, alternatively the coding sequence for a nuclease or nickase fusion may be located on another vector or may be integrated into the cellular genome or the nuclease or nickase fusion may be delivered to the cell as a protein.

As with method 200 supra, the cells of interest may be any cells, including bacterial, yeast and animal (including mammalian) cells. These cells are provided and are transformed with the library of editing vectors 305. Again, transformation is intended to generically include a variety of art-recognized techniques for introducing an exogenous nucleic acid sequence (e.g., an engine and/or editing vector) into a target cell. Once transformed 305, the cells are allowed to recover and selection optionally is performed to select for cells transformed with the editing vector, which most often comprises a selectable marker. As described above, drug selectable markers such as gentamicin, bleomycin, streptomycin, puromycin, hygromycin, blasticidin, and G418 or other selectable markers may be employed. At a next step, conditions are provided such that editing takes place and the cells are incubated to allow for transcription and translation of nucleic acids and proteins, respectively 307, leading to cell editing.

Following editing and incubation 307 where the cells have produced nucleic acids and proteins (including those resulting from the intended edits), in step 302 the cells are fixed and permeabilized. Fixation immobilizes antigens while retaining cellular and subcellular structures and permeabilization allows for access of the nucleic acid-tagged antibodies to the cells and subcellular compartments. The fixation and permeabilization methods used depends on the cell type and on the sensitivity of the analyte being detected and the antibody. Fixation typically is performed using crosslinking reagents such as aldehydes, such as paraformaldehyde, DSP (dithiobis(succinimidyl propionate), DST (disuccinimidyl tartrate) or organic solvents. Acetones are known to do the double duty of fixation and permeabilization. (For fixation and permeabilization techniques, see, e.g., Fox, et al, J. Histochem, 33:845-33 (1985); Coombs, et al., NAR, 27:e12 (1999); Do, et al., Clin. Chem., 59:1376-83 (2013); and Gatta, et al., Eur. J. Histochem., 115:481-86 (2012).)

Once the cells have been fixed and permeabilized 302, nucleic acid-tagged probes—here, nucleic acid-tagged antibodies to intracellular proteins, where each different antibody is tagged with a different nucleic acid tag—are added to the population of edited cells in bulk and the cells are incubated to allow the tagged probes to bind to intracellular proteins in the population of cells 304. In the present exemplary embodiment, the analyte is an intracellular protein; however, it should be understood by one of ordinary skill in the art given the present disclosure that the analyte may be any intracellular analyte for which there is a specific probe. Similarly, the analyte probe in the present embodiment is an antibody; however, the probe may be an aptamer or other ligand that binds specifically to an intracellular analyte and can be conjugated to a nucleic acid tag.

Following incubation with the tagged probes (e.g., nucleic acid-tagged antibodies), the cells are washed to remove unbound antibodies 306, and the edited cells are singulated into partitions 309 then lysed 311. Again, the partitions may be droplets or gel beads as described in relation to FIG. 8A or the partitions may be wells as described in relation to FIG. 8B, both of which are described in detail infra. After the cells are lysed, the cross-linking of proteins, nucleic acids and other macromolecules is reversed 308 by, e.g., treatment in salt or temperatures up to 65° C. or treatment with a reducing agent such as DTT (dithiothreitol) or BME (beta-mecaptoethanol). Once the cells are lysed 311 and cross-linking is reversed 308 to release the nucleic acids present in each cell, barcoded analyte capture primers (or analyte capture primers and barcoded template switching oligonucleotides as described in detail in relation to FIGS. 6A-6F) and barcoded cassette capture primers (or cassette capture primers and barcoded template switching oligonucleotides as described in detail in relation to FIGS. 6A-6F) are added 313. Note—as described supra—in some implementations the barcoded analyte capture primers and barcoded cassette capture primers or analyte capture primers, cassette capture primers and barcoded template switching oligonucleotides may be added to the partitions prior to the cells being added to the partitions and lysed, such as shown in FIG. 8A. Also as described supra, the cassette capture primers and analyte capture primers may be located on a single molecule.

The barcoded cassette capture primers and barcoded analyte capture primers in each partition comprise a different, unique cellular barcode and thus the nucleic acids (including nucleic acid tags from the nucleic acid-tagged antibodies against intracellular proteins) from the singulated cell in each partition is labeled with this unique cellular barcode. After the barcoded analyte capture primers and barcoded cassette capture primers are added, DNA copies or cDNAs are created from the nucleic acid tags on the antibodies that bound to the intracellular markers and from the editing cassettes present in the cell 315 using a combination of one or more of the processes of priming, transcription or reverse transcription, extension and amplification. After the copies are synthesized, they are pooled 317 and sequenced 319. Because each partition comprises a template switching oligonucleotide with a unique cellular barcode, each nucleic acid from each nucleic acid tag and each nucleic acid from each editing cassette transcript from each cell is tagged with this unique cellular barcode; thus, the nucleic acids representing the nucleic acid tags and the nucleic acids representing the editing cassettes from each partition can be correlated 321.

FIG. 3B is a simplified depiction of the process of FIG. 3A and is similar to the process depicted in FIG. 2B. At left in FIG. 3B is a pool of editing vectors (330, 332, 334, 336, and 338), where each editing vector comprises a different editing cassette (e.g., a gRNA and repair template pair; in FIG. 3B, editing vector 330 comprises editing cassette A, editing vector 332 comprises editing cassette B, editing vector 334 comprises editing cassette C, editing vector 336 comprises editing cassette D, editing vector 338 comprises editing cassette E) and the editing cassette optionally comprises a barcode (or more than one barcode) that uniquely identifies the intended edit to be made by the gRNA and repair template pair. If the editing cassette does not comprise a unique barcode, the editing cassette itself serves as the “barcode.” At step 331 a population of cells is transformed with the pool of editing vectors and the conditions are provided to promote nucleic acid-guided nuclease or nickase fusion editing in the cells, producing a genome-edited pool of cells (e.g., cell 340 (blue) edited by editing vector 330; cell 342 (red) edited by editing vector 332; cell 344 (aqua) edited by editing vector 334; cell 346 (green) edited by editing vector 336 and cell 348 (orange) edited by editing vector 338). At step 337, the cells are fixed and permeabilized and nucleic acid-tagged antibodies are added to the edited cells and allowed to bind to intracellular proteins in the cells. Here, three different antibodies each with its own nucleic acid tag are added, where the different antibodies are labeled a (bound to intracellular proteins in cell 351), R (bound to intracellular proteins in cell 353) and 7 (bound to intracellular proteins in cell 355).

At step 339, the cells are washed to remove unbound antibodies, singulated into partitions 360, lysed and cross-linking is reversed. Also at step 339, barcoded analyte capture primers and barcoded cassette capture primers (or in an alternative embodiment, analyte capture primers, cassette capture primers and barcoded template switching oligonucleotides) are added to the lysed cells. Partition 380 comprises cell 350 with barcode 1; partition 381 comprises cell 351 (with the a antibody) with barcode 2; partition 384 comprises cell 354 with barcode 3; partition 385 comprises cell 353 (with the R antibody) with barcode 4 and partition 387 comprises cell 355 (with the 7 antibody) with barcode 5.

At step 351, DNA copies or cDNAs are prepared from the nucleic acid tags and the editing cassettes, the barcoded DNA copies or cDNAs from the partitions are pooled and finally the copies of the nucleic acid tags representing intracellular proteins are correlated with the editing cassettes present in each cell. The nucleic acids of group 370 (blue) are associated with editing cassette A and cellular barcode 1; the nucleic acids of group 372 (red) are associated with editing cassette B, cellular barcode 2 and the a nucleic acid-tagged antibody (there is 1 tagged sequence); the nucleic acids of group 374 (aqua) are associated with editing cassette C and cellular barcode 3; the nucleic acids of group 376 (green) are associated with editing cassette D, cellular barcode 4 and the R nucleic acid-tagged antibody (there are 2 tagged sequences); and the nucleic acids of group 378 (orange) are associated with editing cassette E, cellular barcode 5 and the 7 nucleic acid-tagged antibody (there are 3 tagged sequences).

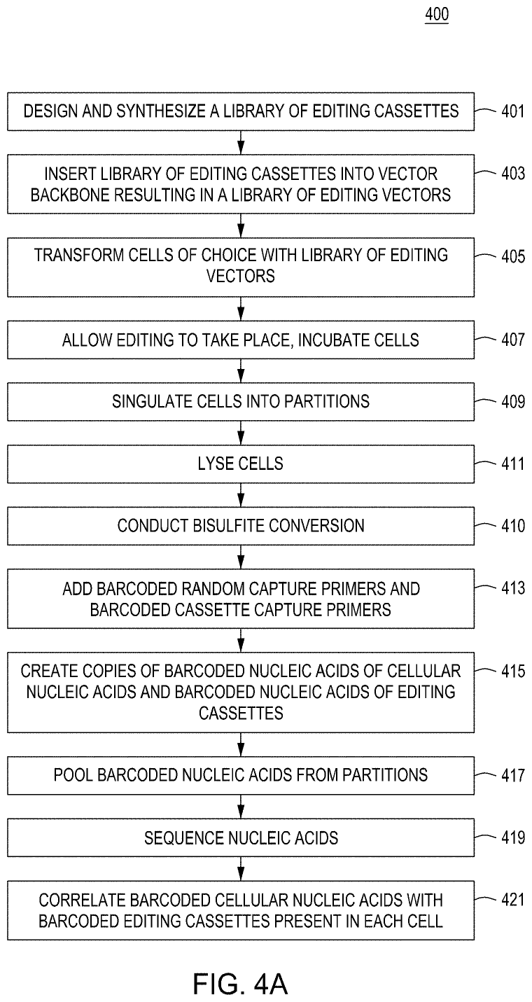

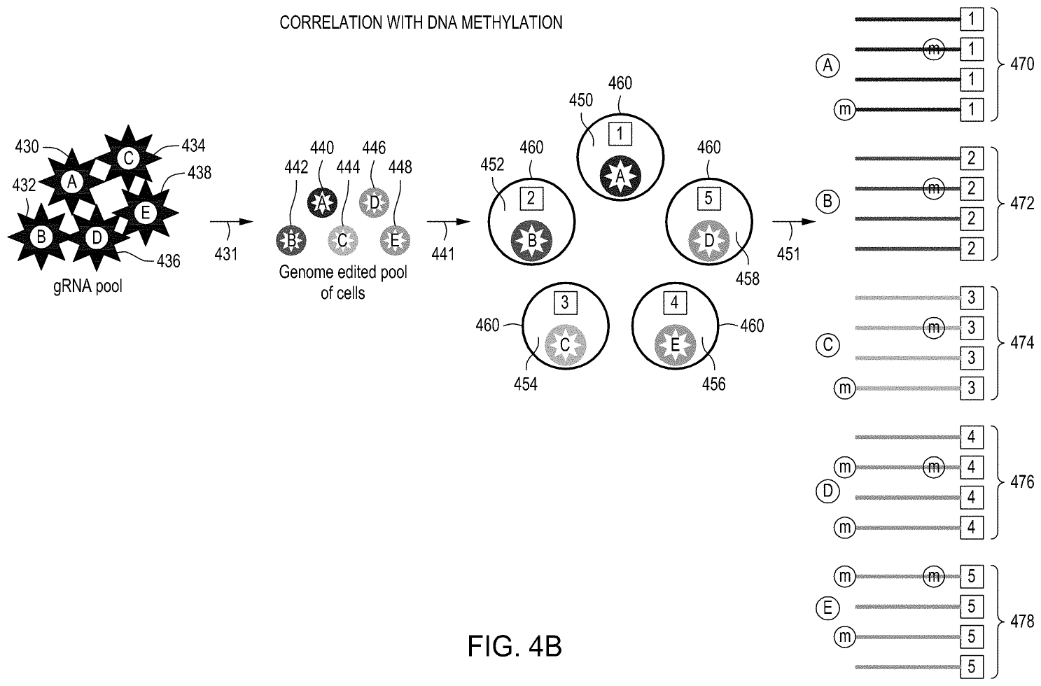

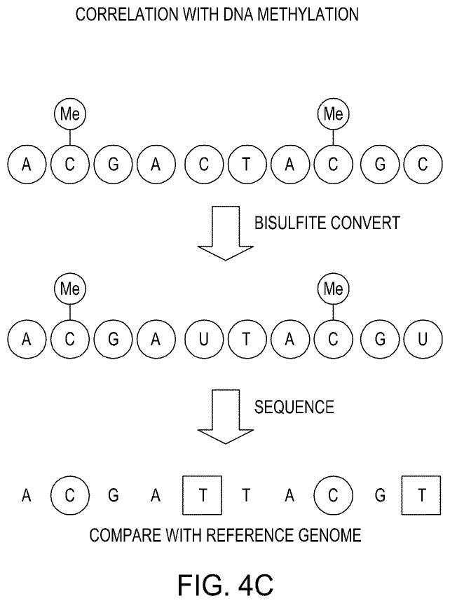

Methylation Profile FIG. 4A is a simple process diagram for multiplexed nucleic acid-guided nuclease or nickase fusion editing in a population of cells and determining the genomic DNA methylation profile resulting from one or more edits in individual cells in the population using barcoded cassette capture primers to capture editing cassettes and barcoded product capture primers (here, barcoded random capture primers) to capture nucleic acids from each cell. DNA methylation is a biological process by which methyl groups are added to a DNA molecule. Methylation can change the activity of a DNA segment without changing the sequence. When located in a gene promoter, DNA methylation typically acts to repress gene transcription. In mammals, DNA methylation is essential for normal development and is associated with a number of key processes including genomic imprinting, X-chromosome inactivation, repression of transposable elements, aging, and carcinogenesis. Two of DNA's four bases, cytosine and adenine, can be methylated. Cytosine methylation is widespread in both eukaryotes and prokaryotes, though the rate of cytosine DNA methylation can differ greatly between species. Method 400, similar to methods 100, 200 and 300, utilizes various combinations of one or more of the processes of priming, transcription or reverse transcription, extension and amplification.

In a first step 401, a library of editing cassettes comprising paired gRNAs and repair templates is designed and synthesized. In addition to paired gRNAs and repair templates, the editing cassettes preferably comprise additional sequences such as one or more priming sequences that can be used to amplify the editing cassette; one or more editing cassette barcodes, which are used to uniquely identify the intended edit to be made by the gRNA and repair template pair; and/or a capture sequence, where the capture sequence facilitates capture of the editing cassette by the cassette capture primers when analyzing the edit/DNA methylation profile relationship. Once designed and synthesized 401, the library of editing cassettes is amplified, purified and inserted 403 into a vector backbone, which in some embodiments may already comprise a coding sequence for the nuclease or nickase fusion to produce a library of editing vectors. Alternatively, the coding sequence for the nuclease or nickase fusion may be located on another vector or may be integrated into the cellular genome. In yet another alternative, the nuclease or nickase fusion may be delivered to the cell as a protein. The vectors chosen for the methods herein will vary depending on the type of cells being edited as described supra.

Cells of choice are provided and are transformed with the library of editing vectors 405. Once transformed 405, the cells are allowed to recover and selection optionally is performed to select for cells transformed with the editing vector, which most often comprises a selectable marker. At a next step 407, conditions are provided such that editing takes place and the cells are incubated to allow for transcription and translation of nucleic acids and proteins, respectively 407. Once editing is complete, the edited cells are singulated into partitions 409 and lysed 411. As described supra in relation to methods 100, 200 and 300, the partitions may be droplets or gel beads as described in relation to FIG. 8A or the partitions may be wells as described in relation to FIG. 8B, both of which are described in detail infra. Once the cells are lysed to release the nucleic acids present in each cell, the cellular nucleic acids are treated to “bisulfite conversion” 410, a technology based on the chemical conversion of unmethylated cytosine residues to uracil residues. Methylated cytosine residues are protected from this conversion, allowing determination of DNA methylation at the single nucleotide level. FIG. 4C depicts this process.

Once the cellular nucleic acids in the lysate have been converted by bisulfite 410, barcoded random capture primers (or the random capture primers and barcoded template switching oligonucleotides as described in detail in relation to FIGS. 6A-6F) and barcoded cassette capture primers (or the cassette capture primers and barcoded template switching oligonucleotides as described in detail in relation to FIGS. 6A-6F) are added 413. Again, please note that in some implementations the barcoded random capture primers and barcoded cassette capture primers may be added to the partitions prior to the cells being added to the partitions and lysed, such as shown in FIG. 8A. As in the methods described supra, the template switching oligonucleotides in each partition comprise a different, one or more unique cellular barcodes and thus the nucleic acids from the singulated cell in each partition is labeled with these unique cellular barcodes. After the barcoded random capture primers and barcoded cassette capture primers are added, DNA copies or cDNAs are created from the cellular nucleic acids and from the editing cassettes present in the cell 415 using a combination of one or more of the processes of priming, reverse transcription or transcription, extension and amplification.

After the DNA copies are synthesized, they are pooled 417 and sequenced 419. Because each partition comprised barcoded cassette capture primers and barcoded random capture primers with a unique cellular barcode, each nucleic acid from each cellular nucleic acid and each nucleic acid from each editing cassette transcript from each cell is tagged with this unique cellular barcode; thus, the nucleic acids representing the cellular nucleic acids and the nucleic acids representing the editing cassettes from each partition can be correlated 421. Once correlated, the cellular nucleic acids further are compared to, e.g., a reference sequence so as to determine which cytosine residues were not methylated. Unmethylated cytosines will appear as thymine residues (or adenine complement) upon bisulfite conversion and DNA copy or cDNA creation.

FIG. 4B is a simplified depiction of the process of FIG. 4A. At left in FIG. 4B is a pool of editing vectors (430, 432, 434, 436, and 438), where each editing vector comprises a different editing cassette (e.g., a gRNA and repair template pair; in FIG. 4B, editing vector 430 comprises editing cassette A, editing vector 432 comprises editing cassette B, editing vector 434 comprises editing cassette C, editing vector 436 comprises editing cassette D, editing vector 438 comprises editing cassette E) and the editing cassette optionally comprises a barcode that uniquely identifies the intended edit to be made by the gRNA and repair template pair. If the editing cassette does not comprise one or more unique barcodes, the editing cassette itself serves as the “barcode.” At step 431 a population of cells is transformed with the pool of editing vectors and the conditions are provided to promote nucleic acid-guided nuclease or nickase fusion editing in the cells, producing a genome-edited pool of cells (e.g., cell 440 (blue) edited by editing vector 430; cell 442 (red) edited by editing vector 432; cell 444 (aqua) edited by editing vector 434; cell 446 (green) edited by editing vector 436; and cell 448 (orange) edited by editing vector 438).

At step 441 the cells are singulated into partitions 460 and treated to bisulfite conversion, which converts unmethylated cytosine residues in the cellular nucleic acids to uracil residues and leaves methylated cytosine residues untouched. Once bisulfite conversion has taken place, barcoded random capture primers and barcoded cassette capture primers (or random capture primers, cassette capture primers and barcoded template switching oligonucleotides) are added to each partition. Partition 450 comprises cell 440 with barcode 1; partition 452 comprises cell 442 with barcode 2; partition 454 comprises cell 444 with barcode 3; partition 456 comprises cell 446 with barcode 4; and partition 458 comprises cell 448 with barcode 5.

At step 451, the cells are lysed, DNA copies or cDNAs are prepared of the cellular nucleic acids and the editing cassettes, the barcoded DNA copies are pooled, and finally the nucleic acids of the cellular nucleic acids are correlated with the editing cassettes present in each cell. The nucleic acids of group 470 (blue) are associated with editing cassette A, cellular barcode 1 and two nucleic acids representing cellular nucleic acid sequences with methylated cytosines; the nucleic acids of group 472 (red) are associated with editing cassette B, cellular barcode 2 and one nucleic acid representing a cellular nucleic acid sequence with methylated cytosines; the nucleic acids of group 474 (aqua) are associated with editing cassette C, cellular barcode 3 and two nucleic acids representing cellular nucleic acid sequences with methylated cytosines; the nucleic acids of group 476 (green) are associated with editing cassette D, cellular barcode 4 and three nucleic acids representing a cellular nucleic acid sequence with methylated cytosines; and the nucleic acids of group 478 (orange) are associated with editing cassette E, cellular barcode 5 and three nucleic acids representing a cellular nucleic acid sequence with methylated cytosines. In yet another embodiment for correlating nucleic acid-guided editing with the resulting methylation profiles, nucleic acid-tagged antibodies against methylcytosine residues may be employed.

FIG. 4C is a simplified representation of bisulfite conversion of unmethylated cytosine residues to uracil residues. An exemplary sequence 5′-AC(me)GACTAC(me)GC-3′ is converted by bisulfite conversion into 5′-AC(me)GAUTAC(me)GU-3′ (here, the unmethylated cytosine residues that were converted into uracil residues are bolded). When sequenced, the sequence will now be read ACGATTACGT-3′. In FIG. 4C, the circled C's (at bottom) were methylated, and thus were not converted to uracil residues and will “remain” C's whereas the boxed T's were unmethylated cytosine residues that were converted to uracil residues and when reversed transcribed and sequenced are now T's. The errant T's can be identified as C's by comparison to a reference genome.

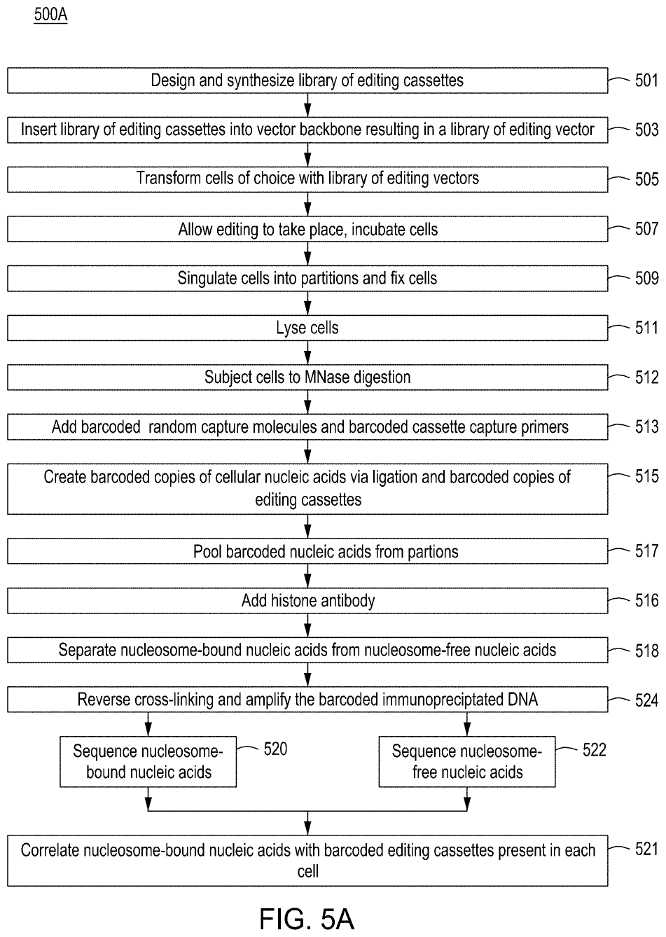

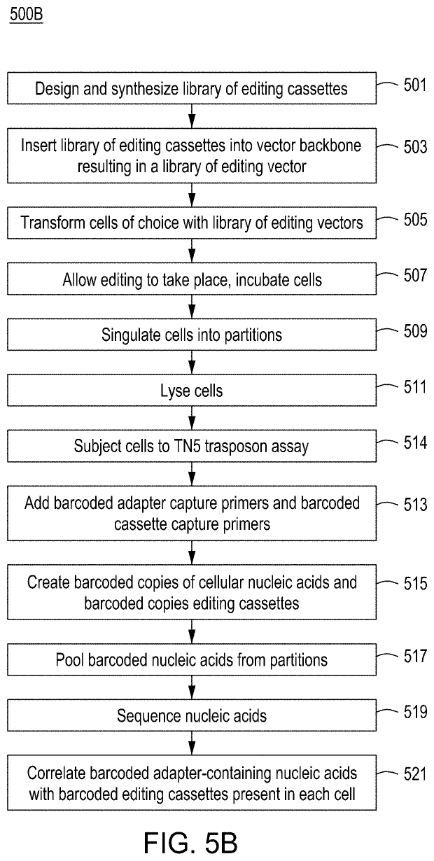

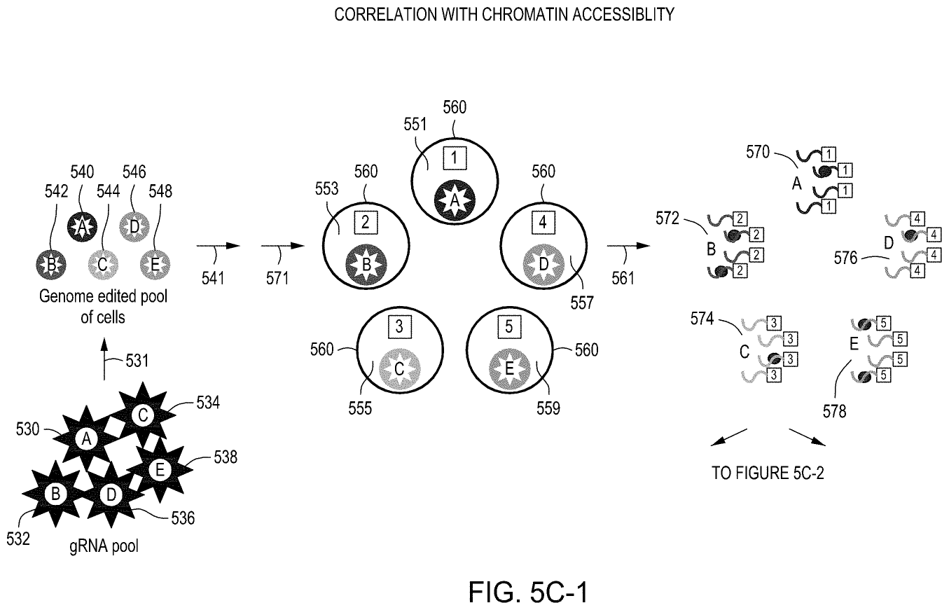

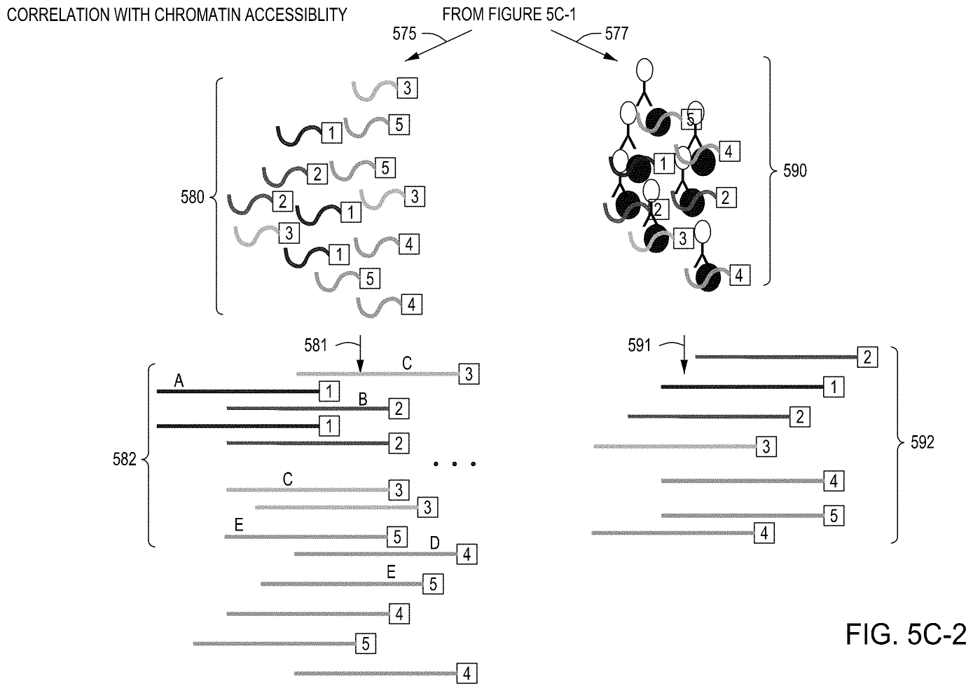

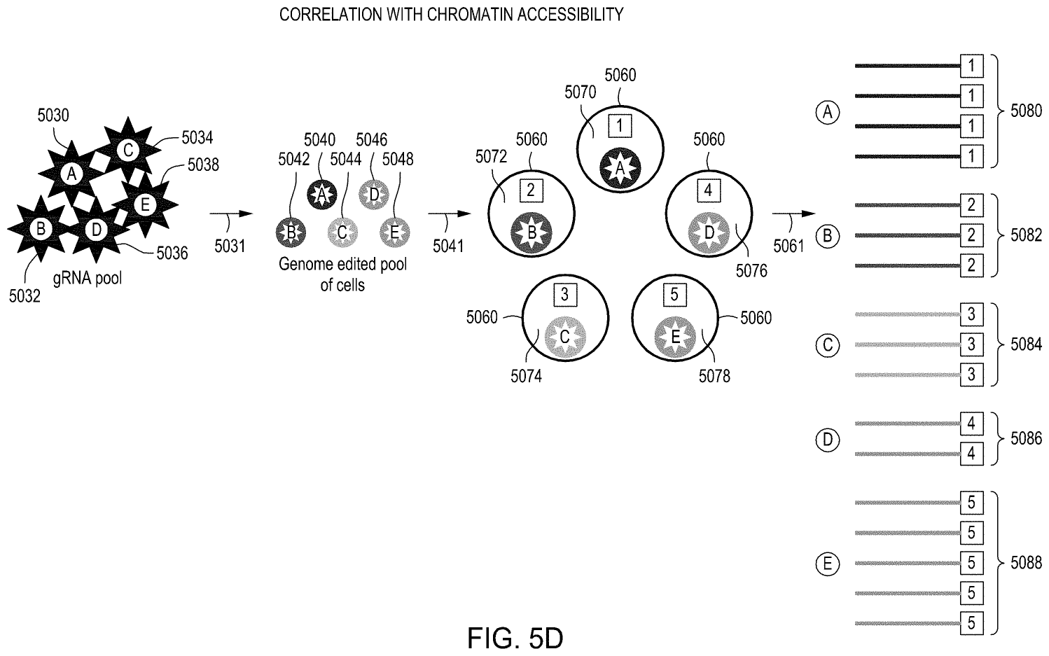

Chromatin Accessibility Profile FIGS. 5A and 5B are simple process diagrams for multiplex nucleic acid-guided nuclease or nickase fusion editing in a population of cells and determining the chromatin accessibility profile resulting from a cellular edit in individual cells in the population using barcoded cassette capture primers to capture editing cassettes and barcoded product capture primers to capture nucleic acids from each cell (or cassette capture primers, product capture primers and barcoded template switch oligonucleotides) that facilitate synthesis of DNA copies or cDNAs from the editing cassettes and cellular nucleic acids. Similar to methods 100, 200, 300 and 400 described supra, the methods depicted in FIGS. 5A and 5B utilize various combinations of one or more of the processes of priming, transcription or reverse transcription, extension and amplification.

Chromatin accessibility—or physical access to DNA—is a highly dynamic property of chromatin that plays an essential role in establishing and maintaining cellular identity. The organization of accessible chromatin across the genome reflects a network of physical interactions through which enhancers, promoters, insulators, transcription binding factors and chromatin-binding factors cooperatively regulate gene expression, which changes dynamically in response to both external stimuli and developmental cues. Current chromatin accessibility assays are used to separate the genome by enzymatic or chemical means and isolate either the accessible or protected locations. The isolated DNA is then quantified using next-gen sequencing. Application of these assays allows for investigation of differential gene expression, cell proliferation, functional diversification and disease development. In the present methods, intended edits to a cellular genome are correlated with changes in the chromatin accessibility profile of the cell.

In a first step 501 of FIG. 5A, a library of editing cassettes comprising paired gRNAs and repair templates is designed and synthesized. In addition to the paired gRNAs and repair templates, the editing cassettes may comprise additional sequences such as a priming sequence, an editing cassette barcode as well as a capture sequence, where the capture sequence facilitates capture of the editing cassette by the cassette capture primers when analyzing the edit/chromatin accessibility profile relationship. Once designed and synthesized 501, the library of editing cassettes is amplified, purified and inserted 503 into a vector backbone which in some embodiments may already comprise a coding sequence for a nuclease or nickase fusion to produce a library of editing vectors. As described in relation to the other methods described supra, instead the coding sequence for the nuclease or nickase fusion may be located on another vector or may be integrated into the cellular genome or the nuclease or nickase fusion may be delivered to the cell as a protein.

The cells of interest may be any eukaryotic cells, including yeast and animal (including mammalian) cells. The cells are provided and are transformed with the library of editing vectors 505. As discussed above, transformation is intended to generically include a variety of art-recognized techniques for introducing an exogenous nucleic acid sequence into a target cell. Once transformed 505, the cells are allowed to recover and selection optionally is performed to select for cells transformed with the editing vector, which most often comprises a selectable marker. At a next step, conditions are provided such that editing takes place and the cells are incubated to allow for transcription and translation of nucleic acids and proteins, respectively 507.

Following editing and incubation 507—where the cells have had changes in chromatin accessibility due to temporally-transcribed nucleic acids (including those resulting from the intended edits)—the edited cells are singulated into partitions and fixed (e.g., cross-linked) 509 then lysed 511. The partitions may be droplets or gel beads as described in relation to FIG. 8A or the partitions may be wells as described in relation to FIG. 8B, both of which are described in detail infra.

After the cells are lysed 511, the cells are subjected to MNase digestion 512. MNase (Micrococcal nuclease) preferentially digests DNA in internucleosomal (linker) regions of the chromatin and partial digestion with MNase reveals periodic spacing of assembled nucleosomes. MNase digestion has been applied to study chromatin structure in a low-throughput manner, e.g., with tiled microarrays; however, currently and in the present embodiment, MNase digestion is combined with next-gen sequencing (e.g., MNase-seq) for genome-wide characterization of average nucleosome occupancy and positioning in a qualitative and quantitative manner. In a typical MNase-seq experiment, mononucleosomes are extracted by extensive MNase treatment of chromatin that has been crosslinked with formaldehyde. In the present method depicted in FIG. 5A, following MNase digestion 512, barcoded random capture molecules (or random capture molecules and barcoded template switching oligonucleotides as described in detail in relation to FIGS. 6A-6F) and barcoded cassette capture primers (or cassette capture primers and barcoded template switching oligonucleotides as described in detail in relation to FIGS. 6A-6F) are added 513 to the MNase-treated cell lysate.

As in embodiments described supra, the barcoded cassette capture primers and barcoded random capture molecules in each partition comprise a different, unique cellular barcode and thus the cellular nucleic acids from the singulated cell in each partition is labeled with this unique cellular barcode. After the barcoded random capture molecules and barcoded cassette capture primers are added 513, the random capture molecules are ligated to cellular nucleic acids and DNA copies or cDNAs are created from the editing cassettes present in the cell 515 using a combination of one or more of the processes of ligation, priming, transcription or reverse transcription, extension and amplification. After the random capture molecules are ligated to cellular nucleic acids and DNA copies of these cellular nucleic acids and DNA copies of the editing cassettes are synthesized 515, the nucleic acids from each partition are pooled 517 and an antibody against histones is added 516 to the nucleic acids to tag and allow separation of cellular nucleic acids associated with histones from cellular nucleic acids that are not associated with histones. After binding of the histone antibodies 516, nucleosome-bound nucleic acids are separated 518 from nucleosome-free nucleic acids by, e.g., immunoprecipitation.

Following separation 518, the cross-linking (e.g., fixing) of the cells is reversed and the barcoded immunoprecipitated DNA is amplified. Cross-linking is discussed in relation to FIG. 3A supra. The nucleosome-bound nucleic acids and non-nucleosome-bound nucleic acids from each partition are sequenced 520, 522. As in earlier embodiments, because each partition comprises barcoded cassette capture primers and barcoded random capture primers with a unique cellular barcode, nucleic acid copies of the nucleic acids in each cell and nucleic acid copies from the editing cassette transcripts present in each cell are tagged with the unique cellular barcode (or more than one unique cellular barcode); thus, the nucleic acids representing the nucleosome-bound nucleic acids and the nucleic acids representing the editing cassettes from each partition can be correlated 521. Note that the principles behind chromatin immunoprecipitation have been extended to look at other protein nucleic acid associations other than histones, including transcription factor binding using TF-specific antibodies, methylation using antibodies to methyl-cytosine residues, etc.; thus, the present embodiment may be extended to these analyses.

In the method depicted in FIG. 5B, instead of MNase digestion the cells are subjected to a T5 transposon assay. This technique, known as ATAC-seq, is the most current method for probing open chromatin and is based on the ability of hyperactive Tn5 transposase to fragment DNA and integrate into active regulatory regions in vivo. During ATAC-seq, cellular nucleic acids are tagged (i.e., in a process called “tagmentation”) with sequencing adapters by purified Tn5 transposase. Due to steric hindrance, the majority of adapters are integrated into regions of accessible chromatin (see, e.g., Tsompana and Buck, Epigenetics and Chromatin, 7:33 (2014)).

In the method depicted in FIG. 5B, a library of editing cassettes comprising paired gRNAs and repair templates is designed and synthesized 501. In addition to the paired gRNAs and repair templates, the editing cassettes may comprise additional sequences such as a priming sequence, an editing cassette barcode as well as a capture sequence, where the capture sequence facilitates capture of the editing cassette by the cassette capture primers when analyzing the edit/chromatin accessibility profile relationship. Once designed and synthesized 501, the library of editing cassettes is amplified, purified and inserted 503 into a vector backbone which in some embodiments may already comprise a coding sequence for a nuclease or nickase fusion to produce a library of editing vectors. As described in relation to the other methods described supra, instead the coding sequence for the nuclease or nickase fusion may be located on another vector or may be integrated into the cellular genome or the nuclease or nickase fusion may be delivered to the cell as a protein.

As with the method depicted in FIG. 5A, the cells of interest may be any eukaryotic cells, including yeast and animal (including mammalian) cells. The cells are provided and are transformed with the library of editing vectors 505. As discussed above, transformation is intended to generically include a variety of art-recognized techniques for introducing an exogenous nucleic acid sequence into a target cell. Once transformed 505, the cells are allowed to recover and selection optionally is performed to select for cells transformed with the editing vector, which most often comprises a selectable marker. At a next step, conditions are provided such that editing takes place and the cells are incubated to allow for transcription and translation of nucleic acids and proteins, respectively 507.

Following editing and incubation 507—where the cells have had changes in chromatin accessibility due to temporally-transcribed nucleic acids (including those resulting from the intended edits)—the edited cells are singulated into partitions 509 then lysed 511. The partitions may be droplets or gel beads as described in relation to FIG. 8A or the partitions may be wells as described in relation to FIG. 8B, both of which are described in detail infra.

After the cells are lysed, the cell lysate is subjected to a TN5 transposon assay 514 where Tn5 transposase is used to fragment DNA and integrate into active regulatory regions in vivo. During this process, cellular nucleic acids are tagged with sequencing adapters by purified Tn5 transposase. The adapters used to tag the cellular nucleic acids may include barcodes and one or more priming sequences. Following T5 transposon digestion 514, barcoded adapter capture primers (or adapter capture primers and barcoded template switching oligonucleotides as described in detail in relation to FIGS. 6A-6F) and barcoded cassette capture primers (or cassette capture primers and barcoded template switching oligonucleotides as described in detail in relation to FIGS. 6A-6F) are added 513 to the T5 transposon-treated cell lysate. The adapter capture primers comprise sequences complementary to the adapters inserted by the Tn5 transposase into the regions of accessible chromatin in the cellular nucleic acids.

After the barcoded adapter capture primers and barcoded cassette capture primers are added 513, DNA copies or cDNAs are created from the cellular nucleic acids and from the editing cassettes present in the cell 515 using a combination of one or more of the processes of priming, reverse transcription or transcription, extension and amplification. After the DNA copies are synthesized 515, the nucleic acids from each partition are pooled 517. After pooling, the population of nucleic acids is sequenced 519 and the nucleic acids representing the adapter-tagged nucleic acids and the nucleic acids representing the editing cassettes from each partition can be correlated 521.

FIGS. 5C-1 and 5C-2 are a simplified depiction of the method depicted in FIG. 5A. At left in FIG. 5C-1 is a pool of editing vectors (530, 532, 534, 536, and 538), where each editing vector comprises a different editing cassette (e.g., a gRNA and repair template pair; in FIG. 5C-1, editing vector 530 comprises editing cassette A, editing vector 532 comprises editing cassette B, editing vector 534 comprises editing cassette C, editing vector 536 comprises editing cassette D, editing vector 538 comprises editing cassette E) and the editing cassette optionally comprises a barcode that uniquely identifies the intended edit to be made by the gRNA and repair template pair. If the editing cassette does not comprise a unique barcode, the editing cassette itself serves as the “barcode.” At step 531 a population of cells is transformed with the pool of editing vectors and the conditions are provided to promote nucleic acid-guided nuclease or nickase fusion editing in the cells, producing a genome-edited pool of cells (e.g., cell 540 (blue) edited by editing vector 530; cell 542 (red) edited by editing vector 532; cell 544 (aqua) edited by editing vector 534; cell 546 (green) edited by editing vector 536; and cell 548 (orange) edited by editing vector 538).

At step 541, the cells are singulated into partitions 560, lysed, and treated with MNase, and at step 571, barcoded random capture primers and barcoded cassette capture primers (or random capture primers, cassette capture primers and barcoded template switching oligonucleotides) are added to the cell lysate and DNA copies or cDNAs are created. Partition 551 comprises cell lysate from cell 540 that has been treated with MNase and is labeled with barcode 1; partition 553 comprises cell lysate from cell 542 that has been treated with MNase and is labeled with barcode 2; partition 555 comprises cell lysate from cell 544 that has been treated with MNase and is labeled with barcode 3; partition 557 comprises cell lysate from cell 546 that has been treated with MNase and is labeled with barcode 4; and partition 559 comprises cell lysate from cell 548 that has been treated with MNase and is labeled with barcode 5. At step 561, the nucleic acids from each partition are pooled and an antibody against histone proteins is added to the pooled nucleic acids. The histone-bound antibodies (and the nucleic acids associated therewith, shown as nucleic acid profiles 570, 572, 574, 576 and 578) are immunoprecipitated thereby separating 575, 577 the nucleosome-free cellular nucleic acids 580 from the nucleosome-bound nucleic acids 590.

The nucleosome-free cellular nucleic acids 580 and nucleosome-bound nucleic acids 590 are sequenced 581, 591 and the cellular nucleic acids representing the nucleosome-free nucleic acids and the editing cassettes (and thus the edits) 582 and nucleosome-bound nucleic acids 592 are correlated. Note that nucleic acids of group 570 (blue) are associated with editing cassette A, cellular barcode 1 and have one nucleic acid sequence associated with a nucleosome; the nucleic acids of group 572 (red) are associated with editing cassette B, cellular barcode 2 and have two nucleic acid sequences associated with a nucleosome; the nucleic acids of group 574 (aqua) are associated with editing cassette C, cellular barcode 3 have one nucleic acid sequence associated with a nucleosome; the nucleic acids of group 576 (green) are associated with editing cassette D, cellular barcode 4 have two nucleic acid sequences associated with a nucleosome; and the nucleic acids of group 578 (orange) are associated with editing cassette E, cellular barcode 5 and have one nucleic acid sequence associated with a nucleosome.

FIG. 5D is a simplified depiction of the method depicted in FIG. 5B. At left in FIG. 5D is a pool of editing vectors (5030, 5032, 5034, 5036, and 5038), where each editing vector comprises a different editing cassette (e.g., a gRNA and repair template pair; in FIG. 5D, editing vector 5030 comprises editing cassette A, editing vector 5032 comprises editing cassette B, editing vector 5034 comprises editing cassette C, editing vector 5036 comprises editing cassette D, editing vector 5038 comprises editing cassette E) and the editing cassette optionally comprises a barcode (or more than one barcode) that uniquely identifies the intended edit to be made by the gRNA and repair template pair. If the editing cassette does not comprise a unique barcode, the editing cassette itself serves as the “barcode.” At step 5031 a population of cells is transformed with the pool of editing vectors and the conditions are provided to promote nucleic acid-guided nuclease or nickase fusion editing in the cells, producing a genome-edited pool of cells (e.g., cell 5040 (blue) edited by editing vector 5030; cell 5042 (red) edited by editing vector 5032; cell 5044 (aqua) edited by editing vector 5034; cell 5046 (green) edited by editing vector 5036; and cell 5048 (orange) edited by editing vector 5038).

At step 5041 the cells are singulated into partitions, lysed and subjected to a TN5 transposon assay. During TN5 transposon digestion, cellular nucleic acids are tagged (i.e., in a process called “tagmentation”) with sequencing adapters by TN5 transposase. Due to steric hindrance, the majority of adapters are integrated into regions of accessible chromatin. After TN5 transposase digestion and tagmentation, barcoded adapter capture primers and barcoded cassette capture primers are added to the Tn5 digested cell lysates and nucleic acids are created from the cellular nucleic acids. Partition 5070 comprises Tn5 transposon-treated cell lysate 5040 with barcode 1; partition 5072 comprises Tn5 transposon-treated cell lysate 5042 with barcode 2; partition 5074 comprises Tn5 transposon-treated cell lysate 5044 with barcode 3; partition 5076 comprises Tn5 transposon-treated cell lysate 5046 with barcode 4; and partition 5078 comprises Tn5 transposon-treated cell lysate 5048 with barcode 5. At step S061, the barcoded nucleic acids are pooled and sequenced and the adapter-tagged cellular nucleic acids are correlated with the editing cassettes present in each cell. The adapter-tagged nucleic acids of group 5080 (blue) are associated with editing cassette A and cellular barcode 1; the adapter-tagged nucleic acids of group 5082 (red) are associated with editing cassette B and cellular barcode 2; the adapter-tagged nucleic acids of group 5084 (aqua) are associated with editing cassette C and cellular barcode 3; the adapter-tagged nucleic acids of group 5086 (green) are associated with editing cassette D and cellular barcode 4; and the adapter-tagged nucleic acids of group 5088 (orange) are associated with editing cassette E and cellular barcode 5.

Exemplary Embodiment for Creating Barcoded DNA Copies (or cDNAs) of Cellular Nucleic Acids and Editing Cassettes Using Template Switching Oligonucleotides

FIG. 6A depicts the processes of reverse transcription and template switching for cellular nucleic acids to create DNA copies or cDNAs (“DNA copies”) in a cell after random capture primers and template switching oligonucleotides have been combined with the lysate of an individual cell (e.g., step 113 of FIG. 1A). At top left of FIG. 6A is a template switching oligonucleotide (TSO) 622 attached to a bead 620. TSO comprises from left (5′) to right (3′) a read 1 sequencing primer binding sequence 623, a cellular barcode 624, a unique molecular identifier 625 and a TSO sequence 626 comprising a poly-dG tract 627. Cellular barcode 624 is unique to each partition whether the partition is a droplet, gel bead or a well, and the unique molecular identifiers 625 comprise a tract of nucleotides coupled with a particular cellular barcode where each unique molecular identifier coupled with a particular cellular barcode is different. Cellular barcode 624 facilitates association of DNA copies created from the cellular nucleic acids and editing cassette transcripts originating from a single cell, and the unique molecular identifiers allow tracking of DNA copies originating from a single DNA copy after amplification.

At top right of FIG. 6A is a cellular nucleic acid 628. Below cellular nucleic acids 628 is a random capture primer 621 comprising a priming sequence 629 and a random hybridization sequence 630 that can hybridize to a complementary sequence 630′ in cellular nucleic acid 628. Random priming is an efficient method for copying cellular nucleic acids to produce DNA libraries. Random priming—i.e., using random sequences to hybridize to complementary sequence in the cellular genome—has been used to amplify sequences from an entire genome in a single cell. In a next step, complementary sequence in the cellular nucleic acid 628 and random capture primer 621 are hybridized and a copy is made 631 of cellular nucleic acid 628 resulting in a reverse transcript construct 633. During reverse transcription of the cellular nucleic acid several to many untemplated Cs 632 are added to the 3′ end of the reverse transcript construct 633. Untemplated Cs 632 are capable of hybridizing with the poly-dG tract 627 of TSO 622 (allowing for, e.g., TSO priming). After TSO priming of the cellular nucleic acid 628 and reverse transcript construct 633, the reverse transcript construct 633 is extended from the untemplated Cs 632 to include TSO sequence complement 626′, unique molecular identifier complement 625′, cellular barcode complement 624′, and read 1 sequencing primer binding sequence complement 623′ resulting in an extended cDNA transcript 636.

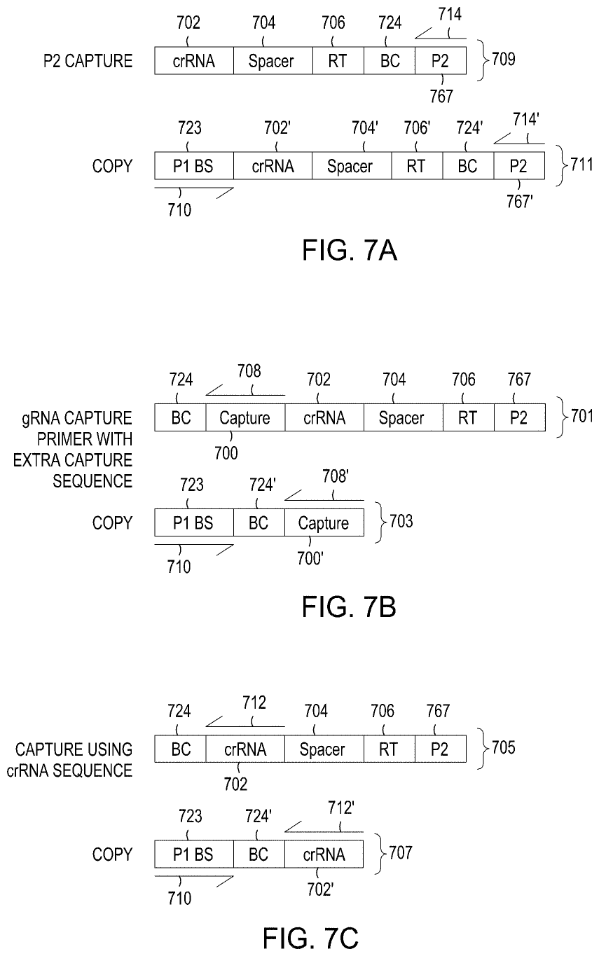

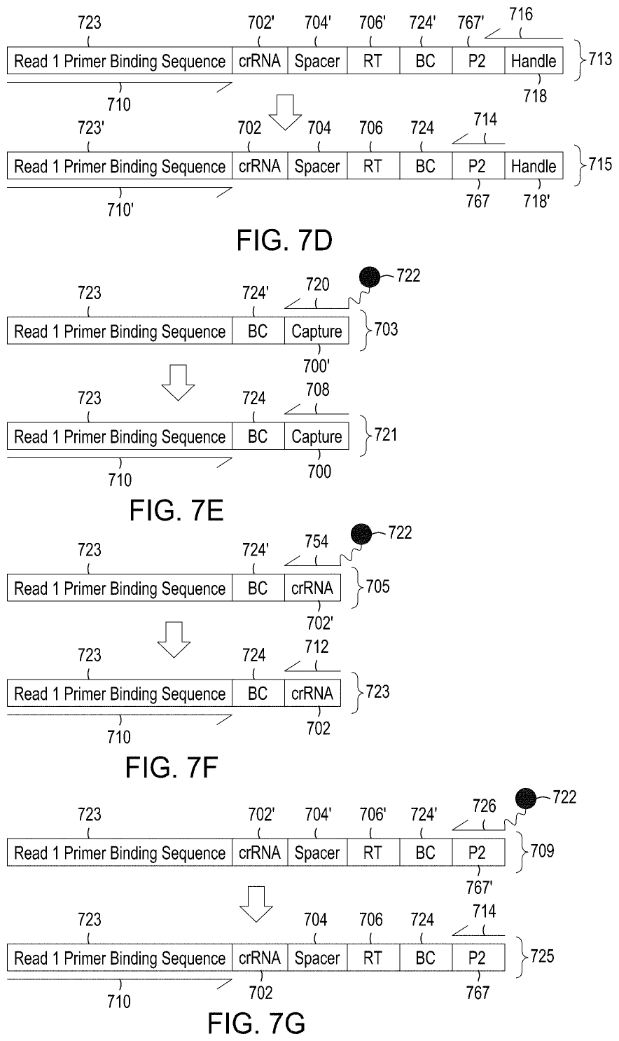

FIG. 6B depicts an exemplary embodiment of the processes of reverse transcription and template switching for editing cassette transcripts in a cell after cassette capture primers and barcoded template switching oligonucleotides have been combined with the lysate of an individual cell (e.g., step 113 of FIG. 1A). (Note that in this FIG. 6B the cassette capture primers detailed in FIG. 7A were used; however, one of ordinary skill in the art given the present disclosure would understand that alternative structures of cassette capture primers may be used, including those detailed in FIGS. 7B and 7C.) At top left of FIG. 6B is a template switching oligonucleotide (TSO) 622 attached to a bead 620. Like the TSO in FIG. 6A, the TSO comprises from left (5′) to right (3′) read 1 sequencing primer binding sequence 623, a cellular barcode 624, a unique molecular identifier 625 and a TSO sequence 626 comprising a poly-dG tract 627. Also as with the TSO depicted in FIG. 6A, cellular barcode 624 is unique to each partition whether the partition is a droplet or a well, and the unique molecular identifiers 625 comprise a tract of nucleotides coupled with a particular cellular barcode where each unique molecular identifier coupled with a particular cellular barcode is different. Cellular barcode 624 facilitates association of the DNA copies created from the cellular nucleic acids and editing cassette transcripts originating from a single cell, and the unique molecular identifiers allow tracking of DNA copies originating from a single DNA copy after amplification.

At top right of FIG. 6B is editing cassette transcript 640 and positioned below editing cassette transcript 640 in this FIG. 6B is cassette capture primer 641 comprising a priming sequence 642 and a cassette capture sequence 644 that is complementary to a sequence associated with the editing cassette (e.g., complementary to part of the editing cassette itself or complementary to a cassette capture sequence). In a next step, the editing cassette transcript 640 and cassette capture primer 641 are hybridized and reverse transcription is performed primed from cassette capture primer 641 resulting in a copy 645 of editing cassette transcript 640. During reverse transcription of editing cassette transcript 640, several to many untemplated Cs 646 are added to the 3′ end of the reverse transcript construct 643. Untemplated Cs 646 are capable of hybridizing with the poly-dG tract 627 of TSO 622 (allowing for, e.g., TSO priming). After TSO priming of editing cassette transcript 640 and reverse transcript construct 643, reverse transcript construct 643 is extended from the untemplated Cs 646 to include TSO sequence complement 626′, unique molecular identifier complement 625′, cellular barcode complement 624′, and read 1 sequencing primer binding sequence complement 623′ resulting in an extended cDNA transcript 648.

FIG. 6C is a depiction of the process of amplification of the duplex-extended DNA copies created from the cellular nucleic acids and editing cassettes. At top is a duplex 660 of the extended DNA transcript 648 and its complement 648′ resulting from copying the editing cassettes present in the cells. The duplex extended nucleic acid 648/648′ comprises from left (5′) to right (3′) read 1 sequencing primer binding sequence 623 (and its complement 623′), cellular barcode 624 (and its complement 624?), a unique molecular identifier 625 (and its complement 625′), a TSO sequence 626 (and its complement 626′), a poly-dG tract 627 (and its complement poly-dC tract 646) (neither shown in this FIG. 6C), the copy of the editing cassette transcript 645 (and its complement 645′), editing cassette complement sequence 644 (and its complement cassette capture sequence 644′), and priming sequence 642 (and its complement 642′). Amplification primer 650 binds to priming sequence 642 and sequencing read amplification primer 652 binds to the complement of read 1 sequencing primer binding sequence 623′ to amplify the duplex extended DNA transcript 648/648?.

At bottom of FIG. 6C is a duplex 662 of the extended DNA transcript 636 and its complement 636′ resulting from copying the nucleic acids in the cell. The duplex extended DNA transcript 636 and its complement 636′ comprises from left (5′) to right (3′) read 1 sequencing primer binding sequence 623 (and its complement 623′), cellular barcode 624 (and its complement 624′), a unique molecular identifier 625 (and its complement 625′), a TSO sequence 626 (and its complement 626′), a poly-dG tract (and its complement poly-dC tract 646) (neither shown in this FIG. 6C), the copy of the nucleic acid 631 (and its complement 631?), random hybridization sequence 630 (and its complement 630′, a poly-A from the mRNA transcript), and priming sequence 629 (and its complement 629′). Amplification primer 654 binds to priming sequence 636 and sequencing read amplification primer 652 binds to the complement of read 1 sequencing primer binding sequence 623′ to amplify extended DNA transcript 636 and its complement 636′.

FIG. 6D is a depiction of size selection of the cellular nucleic acids and editing cassette extended transcripts. The extended DNA transcripts 662 created from copying the cellular nucleic acids in the cell and extended DNA transcripts 660 created from copying the editing cassettes in the cell differ in size.

FIGS. 6E-A and 6E-B are depictions of sequencing library generation for the random cellular nucleic acid- and editing cassette-generated DNA copies where sample indices and P5 and P7 sequencing primer sequences are added to the DNA copies. In FIG. 6E-A, the size-selected DNA duplex created from the cellular nucleic acid extended transcript 662 is seen. Size-selected DNA duplex 662 comprises from left (5′) to right (3′) read 1 sequencing primer binding sequence 623 (and its complement 623′), cellular barcode 624 (and its complement 624′), unique molecular identifier 625 (and its complement 625′), TSO sequence 626 (and its complement 626′), a poly-dG tract (and its complement poly-dC tract 646) (neither shown in this FIG. 6E-A), cellular nucleic acid transcript 631 (and its complement 631?), random hybridization sequence 630 (and its complement 630′ cellular nucleic acid transcript), and priming sequence 629 (and its complement 629′). Enzymatic fragmentation is then performed creating truncated DNA duplex 664 where a portion of random cellular nucleic acid transcript 631 (and its complement 631′) is cleaved, thereby cleaving off a portion of the random capture primer 630 and the cellular nucleic acid sequence complement 630′ as well as the priming sequence 629 (and its complement 629′). Following enzymatic fragmentation, a combination of end repair, A-tailing and ligation of a read 2 sequencing primer binding sequence to the 3′ end of truncated DNA duplex 664 is performed to create DNA duplex 666 with read 1 sequencing primer binding sequence 623 at its 5′ end and read 2 sequencing primer binding sequence 667 at its 3′ end (with the complements thereof 623′ and 667′, respectively).

After ligation of the read 2 sequencing primer binding sequence 667, 667′, DNA duplex 666 is primed with a P5 primer 668 comprising a P5 sequence 669 and a read 1 primer 670, and with a P7/sample index primer 671 comprising a P7 sequence 672, a sample index 673 and a read 2 primer 674. Amplification with P5 primer 668 and P7/sample index primer 671 results in final DNA library constructs 675 created from the cellular mRNAs ready for sequencing on ILLUMINA™'s HiSeq®, MiSeq®, NextSeq, NovoSeq platforms or other ILLUMINA™ sequencing systems. Final DNA library constructs 675 comprise from 5′ to 3′ P5 sequence 669 (and its complement 669′), read 1 sequencing primer binding sequence 623 (and its complement 623′), cellular barcode 624 (and its complement 624′), unique molecular identifier 625 (and its complement 625′), TSO sequence 626 (and its complement 626′), mRNA sequence 631 (and its complement 631′), read 2 sequencing primer binding sequence 667 (and its complement 667′), sample index 673 (and its complement 673′), and P7 sequence 672 (and its complement 672′).

In FIG. 6E-B, the size-selected DNA duplex 660 corresponding to the editing cassettes present in the cell is seen. Size-selected DNA duplex 660 comprises from left (5′) to right (3′) read 1 sequencing primer binding sequence 623 (and its complement 623′), cellular barcode 624 (and its complement 624′), unique molecular identifier 625 (and its complement 625′), TSO sequence 626 (and its complement 626′), a poly-dG tract (and its complement a poly-dC tract 646) (neither shown in this FIG. 6E-B), editing cassette transcript 645 (and its complement 645′), cassette capture sequence 644 (and its complement 644′), and priming sequence 642 (and its complement 642′). In processing of size-selected DNA duplex 660, enzymatic fragmentation is not performed. Instead, two primers are added to size-selected duplex DNA 660: 1) a P5 primer 668 comprising a P5 sequence 669 and a read 1 primer sequence 670; and 2) primer sequence 680 with a sequence 684 complementary to priming sequence 642 and a read 2 sequencing primer binding sequence 683

In a next step, sample indexing is performed using the P5 primer 668 comprising a P5 sequence 669 and a read 1 primer sequence 670 used in the previous step with a P7/sample index primer 671 comprising a P7 sequence 672, a sample index 673 and a read 2 primer 674. Amplification with P5 primer 668 and P7/sample index primer 671 results in final DNA library constructs 690 created from cellular editing cassettes ready for sequencing on any of the ILLUMINA™ sequencing systems. Final DNA library constructs 690 comprise from 5′ to 3′ P5 sequence 669 (and its complement 669′), read 1 sequencing primer binding sequence 623 (and its complement 623′), cellular barcode 624 (and its complement 624′), unique molecular identifier 625 (and its complement 625′), TSO sequence 626 (and its complement 626′), editing cassette sequence 645 (and its complement 645′), cassette capture sequence 644 (and its complement 644′), priming sequence 642 (and its complement 642′), read 2 sequencing primer binding sequence 683 (and its complement 683′), sample index 673 (and its complement 673′), and P7 sequence 672 (and its complement 672′).

Transcriptome analysis is the study of the transcriptome—i.e., the complete set of RNA transcripts that are produced by the genome—under specific circumstances or in a specific cell, using high-throughput methods. Generally, the goal of transcriptome analysis is to identify genes differentially expressed among different conditions, leading to a new understanding of the genes or pathways associated with the conditions. Data obtained from the transcriptome is used in research to gain insight into processes such as cellular differentiation, carcinogenesis, transcription regulation and biomarker discovery among others. FIG. 6F is a depiction of the processes of reverse transcription and template switching for mRNA transcripts to create cDNAs in a cell after poly-dT primers and template switching oligonucleotides have been combined with the lysate of an individual cell (e.g., step 113 of FIG. 1A). At top left of FIG. 6F is a template switching oligonucleotide (TSO) 622 attached to a bead 620. TSO comprises from left (5′) to right (3′) a read 1 sequencing primer binding sequence 623, a cellular barcode 624, a unique molecular identifier 625 and a TSO sequence 626 comprising a poly-dG tract 627. Cellular barcode 624 is unique to each partition whether the partition is a droplet or a well, and the unique molecular identifiers 625 comprise a tract of nucleotides coupled with a particular cellular barcode where each unique molecular identifier coupled with a particular cellular barcode is different. Cellular barcode 624 facilitates association of cDNAs created from the mRNA and editing cassette transcripts originating from a single cell, and the unique molecular identifiers allow tracking of cDNAs originating from a single cDNA after amplification.

At top right of FIG. 6F is an mRNA transcript 694 comprising a poly-A tract at the 3′ end. Below mRNA transcript 694 is a poly-dT primer 693 comprising a priming sequence 629 and a poly-dT tract 692 that can capture the poly-A tracts of mRNAs. In a next step, the mRNA transcript 694 and poly-dT primer 693 are hybridized and a copy is made 631 of mRNA transcript 694 resulting in a reverse transcript construct 695. During reverse transcription of the mRNA transcript several to many untemplated Cs 632 are added to the 3′ end of the reverse transcript construct 695. Untemplated Cs 632 are capable of hybridizing with the poly-dG tract 627 of TSO 622 (allowing for, e.g., TSO priming). After TSO priming of mRNA transcript 694 and reverse transcript construct 695, the reverse transcript construct 695 is extended from the untemplated Cs 632 to include TSO sequence complement 626′, unique molecular identifier complement 625′, cellular barcode complement 624′, and read 1 sequencing primer binding sequence complement 623′ resulting in an extended cDNA transcript 636.

As an alternative to performing whole transcriptome analysis, one can use the methods and compositions described herein to conduct targeted transcriptome analysis; that is, a hybrid capture step is performed to pull down or isolate transcripts of interest following synthesis of the second strand (i.e., reverse transcript 695 in FIG. 6F). Following hybrid capture, library preparation proceeds as with whole transcriptome analysis as described above.

FIGS. 7A-7G depict details of alternative embodiments of editing cassette architectures and the DNA copies resulting therefrom that may be used to correlate mRNAs to editing cassette sequences. FIG. 7A shows the details of the editing cassette architecture 709 that is captured and copied in the process shown in FIG. 6B where a read 2 sequencing primer binding sequence 767 is used to perform an initial amplification of the editing cassette 709 and read 1 and 2 sequencing primers (710 and 714′) are used to perform a second amplification resulting in DNA copy 711. The top figure of FIG. 7A shows the details of an editing cassette 709 comprising from 5′ to 3′: a crispr RNA region 702 (crRNA); a spacer 704 (e.g., the portion of the gRNA that is complementary to the target site being edited); a repair template (RT) 706 which is integrated at the site of nuclease-mediated cleavage (or nickase fusion-mediated cleavage); a barcode region 724 (BC) of the editing cassette which uniquely identifies the editing cassette; and a primer sequence 767 (P2) for amplifying the editing cassette via PCR via primer 714. After the cassette is amplified via primer P2 714, the resulting DNA copy 711 comprises from 3′ to 5′: a primer sequence binding site (P1 BS) for amplifying the DNA copy 723; the complement 702′ to the crispr RNA region 702; the complement 704′ to spacer 704; the complement 706′ to repair template 706; the complement 724′ to barcode region 724 (BC); and the complement 767′ to primer sequence 767 (P2) where primers 710 and 714′ are used to amplify DNA copy 711.

FIG. 7B depicts details of an alternative editing cassette 701 that may be used in the methods described herein, where a primer 708 complementary to all or a portion of a capture sequence 700 is used to perform an initial amplification of editing cassette 701 and a primer 708′ complementary to all or a portion of the complement 700′ of the capture sequence 700 and a read 1 sequencing primer 710 is used to perform a second amplification resulting in DNA 703. The top figure of FIG. 7B shows the details of editing cassette 701 from 5′ to 3′: a barcode region 724 (BC) of the editing cassette which uniquely identifies the editing cassette; a capture region 700 that allows the editing cassette to be captured; a crispr RNA region 702 (crRNA); a spacer 704; a repair template (RT) 706; and a primer sequence 767 (P2). After the cassette is amplified via capture primer 708, the resulting DNA 703 comprises from 3′ to 5′: a primer sequence binding site (P1 BS) for amplifying the DNA 723; the complement 724′ to barcode region 724 (BC); and the complement 700′ to capture sequence 700 where primers 710 and 708′ may then be used to amplify DNA 703. Note in this embodiment the barcode specific to the editing cassette is used to identify the editing cassette as the crRNA, spacer and HA are not present in the DNA.

FIG. 7C depicts details of yet another alternative editing cassette 705 that may be used in the methods described herein wherein a primer 712 complementary to the crispr RNA region 702 is used to perform the initial amplification reaction. Editing cassette 705 comprises from 5′ to 3′: a barcode region 724 (BC); a crispr RNA region 702 (crRNA); a spacer 704; a repair template (RT) 706; and a primer sequence 767 (P2). Editing cassette 705 initially is amplified by primer 712, which is complementary to crispr RNA region 702, resulting in DNA 707 comprising from 3′ to 5′: a primer sequence binding site (P1 BS) for amplifying the DNA 723; the complement 724′ to barcode region 724 (BC); and the complement 702′ to crispr RNA region 702, where primers 710 and 712′ can be used to further amplify DNA 707. As with the embodiment described in relation to FIG. 7B, in this embodiment the barcode specific to the editing cassette is used to identify the editing cassette as the crRNA, spacer and HA are not present in the DNA.

FIG. 7D details one method for enriching for DNAs derived from the editing cassettes of FIG. 7A using nested PCR. DNA 713 comprises from 5′ to 3′: read 1 sequencing primer binding site 723; a sequence 702′ complementary to crispr RNA region 702 (crRNA); a sequence 704′ complementary to spacer 704; a sequence complementary 706′ to repair template (RT) 706; a sequence 724′ complementary to barcode region 724; a sequence 767′ complementary to primer sequence 767 (P2); and a handle 718 which is added to the DNA during a previous amplification. Primer 710 primes at read 1 sequencing primer binding site 723 and primer 716 primes at handle 718 and at least a portion of complementary read 2 sequencing primer binding site 767′, resulting in secondary DNA 715 comprising from 3′ to 5′: a complement 723′ to primer sequence binding site 723; crispr RNA region 702; spacer 704; repair template 706; barcode region 724; and the complement 767 to primer sequence 767′ (P2). Primer 710′ and nested primer 714 are used to amplify DNA 715.

FIG. 7E details one method for enriching for DNAs derived from the editing cassettes of FIG. 7B using hybrid capture. Looking at FIG. 7E, after the editing cassette 701 is captured and amplified via capture primer 708, the resulting DNA 703 comprises from 3′ to 5′: a primer sequence binding site 723; the complement 724′ to barcode region 724 (BC); and the complement 700′ of capture sequence 700. Biotinylated primer 720 comprises a region that is complementary to the complement to capture sequence 700′ and a biotinylated capture moiety 722. Once captured, DNA 721 can be further amplified by primers 710 and 708.

FIG. 7F details one method for enriching for DNAs derived from the editing cassettes of FIG. 7C using hybrid capture. Looking at FIG. 7C, after the editing cassette 705 is captured via the crRNA sequence and amplified via primer 712, the resulting DNA 705 comprises from 3′ to 5′: a primer sequence binding site 723 (P1 BS) for amplifying the DNA; the complement 724′ to barcode region 724 (BC); and the complement 702′ to crispr RNA region 702. Biotinylated primer 754 comprises a region that is complementary to the complementary crispr RNA 702′ and a biotinylated capture moiety 722. Once captured, DNA 723 can be further amplified by primers 710 and 712.

FIG. 7G details one method for enriching for DNAs derived from the editing cassettes of FIG. 7A using hybrid capture (e.g., as opposed to using nested primers as shown in FIG. 7D. Looking at FIG. 7A, after editing cassette 709 is amplified via primer 714, the resulting DNA 711 comprises from 3′ to 5′: a primer sequence binding site (P1 BS) for amplifying the DNA 723; the complement 702′ to a crispr RNA region 702; the complement 704′ to spacer 704; the complement 706′ to repair template 706; the complement 724′ to barcode region 724; and the complement 767′ to read 2 primer sequence 767 (P2). Biotinylated primer 726 comprises a region that is complementary to the complement of read 2 primer sequence 767′ and a biotinylated capture moiety 722. Once captured, DNA 725 can be further amplified by primers 710 and 714.

Modules for Correlating Edits with Cellular Nucleic Acids

Once the desired cell population has gone through a desired number of editing rounds, the cells are singulated into partitions and the cellular nucleic acids and editing cassettes from each cell are captured as described in FIGS. 6A-6F. FIG. 8A and FIG. 8B are simplified diagrams of two embodiments of methods for singulating cells where the methods of FIGS. 6A-6F may be performed resulting in a correlation of the editing cassettes present in the cells and the nucleic acid profile of the cells. In the method shown in FIG. 8A, a Chromium Single Cell Gene Expression Solution with Feature Barcoding technology utilizing gel beads-in-emulsion (GEMs) as commercialized by 10× GENOMICS™ (Pleasanton, Calif.) may be employed. The system partitions thousands of cells into nanoliter-scale GEMs where all generated DNAs share a common barcode.

The methods commercialized by 10× GENOMICS™ rely on microfluidic emulsion technology. In general, microfluidic droplet generation using two immiscible fluids (e.g., an aqueous solution and an oil) that meet at intersecting microchannels with droplets being generated at a junction between the two microfluidic channels has been known in the art for several decades. The terms “droplet” and “emulsion” are used interchangeably herein to refer to an aliquot of one fluid (here, an aqueous solution) in an immiscible carrier fluid (e.g., an oil), with the carrier fluid substantially surrounding the aqueous droplet. The immiscible fluids used in the microchannels may be provided by on-chip wells or off-chip reservoirs and the channels intersect as T-junctions or flow-focusing junctions to form the droplets. A pressure differential is used to control the flow of the fluids at the T-junction or focusing junctions, shearing-off of the aqueous fluid into the immiscible oil flow to create droplets. By adjusting the pressure of the flowing fluids, a pressure difference can be established to shear off droplets of the aqueous solution at a regular frequency as the aqueous solution enters the oil stream, thereby forming droplets in the oil stream. The droplets optionally may be combined with other droplets to effect a reaction between the reactants when the droplets combine. Microfluidic devices or “chips” are available commercially through, e.g., microfluidic ChipShop™ GmbH (Jena, Germany); uFluidix™ (Toronto, Canada); Microflexis™ (Hamburg, Germany); and microLIQUID™ (Gipuzkoa, Spain).

Some embodiments employ liquid droplets and such embodiments are particularly useful in co-partitioning reagents with cells for further reaction and analysis and for co-partitioning of reagents and sample components. Partitioning and co-partitioning of reagents and merging of droplets reduces the complexity of sample material by segregating portions of the sample into different partitions. By segregating reagents, each sample portion (e.g., cell) can be subjected into a different reaction (or barcoded sets of reagents) with thousands, tens of thousands, hundreds of thousands or more reactions taking place in parallel. Further in the case of droplets in an emulsion, allocating individual molecules (e.g., Poisson distribution of cells and/or molecules in droplets) is accomplished by introducing an aqueous stream of fluid comprising the cells or molecules into a flowing stream of a carrier fluid such that droplets are generated at the junction of the two streams. By providing the aqueous molecule-containing (or cell-containing) stream at a certain concentration level, the number of droplets containing molecules (or cells) can be controlled. In the present methods, it is desirable to control the relative flow rates of the aqueous and non-aqueous fluid such that, on average, the droplets contain less than one set of barcoded cassette capture primer/barcoded random capture primer pair per droplet to ensure that any one droplet will comprise either one set of barcoded capture primers or no barcoded capture primers.

The fluid that is immiscible with the aqueous fluid is typically a non-polar hydrophobic fluid such as an oil, e.g., mineral oil, or an organic liquid such as hexadecane. Fluorinated oils are commonly used, with a fluorosurfactant added to stabilize the droplets that are formed. Non-limiting examples of fluorophilic components that can be used in either a surfactant and/or a continuous phase include: perfluorodecalin, perfluoromethyldecalin, perfluoroindane, perfluorotrimethyl bicyclo[3.3.1]nonane, perfluoromethyl adamantine, perfluoro-2,2,4,4-tetra-methylpentane; 9-12C perfluoro amines, e.g., perfluorotripropyl amine, perfluorotributyl amine, perfluoro-1-azatricyclic amines; bromofluorocarbon compounds, e.g., perfluorooctyl bromide and perfluorooctyl dibromide; F-4-methyl octahydroquinolidizine and perfluoro ethers, including chlorinated polyfluorocyclic ethers, perfluoro-4-methylmorpholine, perfluorotriethylamine, perfluoro-2-ethyltetrahydrofuran, perfluoro-2-butyltetrahydrofuran, perfluoropentane, perfluoro(2-methylpentane), perfluorohexane, perfluoro-4-isopropylmorpholine, perfluorodibutyl ether, perfluoroheptane, perfluorooctane, perfluorotripropylamine, perfluorononane, perfluorotributylamine, perfluorodihexyl ether, perfluoro[2-(diethylamino)ethyl-2-(N-morpholino)ethyl]ether, n-perfluorotetradecahydrophenanthrene, and mixtures thereof.

In an embodiment similar to using liquid droplets, the liquid droplets are polymerized into gel beads. The gel matrix forming a bead typically comprises at least one polymer and a linker. A bead may be porous, non-porous, solid, semi-solid, and/or semi-fluidic and in some embodiments, the bead is degradable, dissolvable or disruptable. The gel bead may be a hydrogel bead and is formed from molecular precursors such as a polymeric or monomeric species. Gel beads may be of uniform size or heterogeneous size. In some cases, the diameter of a bead is at least about 1 micrometer (μm), 5 μm, 10 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 400 μm, 500 μm, 1 mm, or greater. Typically, the gel beads are provided as a population or plurality of gel beads having a relatively monodisperse size distribution as it is desirable to provide relatively consistent amounts of reagents within the gel beads.

Gel beads for use herein contain molecular precursors (e.g., monomers or polymers) which form the polymer network via polymerization of the molecular precursors. In some embodiments, a precursor may be an already polymerized species capable of undergoing further polymerization via, for example, a chemical cross-linkage. For example, a precursor may comprise one or more of an acrylamide or a methacrylamide monomer, oligomer, or polymer. In some cases, the gel bead may comprise pre-polymers, which are oligomers capable of further polymerization; for example, polyurethane beads may be prepared using prepolymers. Alternatively, the bead may contain individual polymers that may be further polymerized together. In some embodiments, gel beads may be generated via polymerization of different precursors, such that they comprise mixed polymers, co-polymers, and/or block co-polymers. In some embodiments, the gel bead may comprise covalent or ionic bonds between polymeric precursors (e.g., monomers, oligomers, linear polymers) and other entities. In some aspects, the covalent bonds may be carbon-carbon bonds or thioether bonds. In the present methods, cross-linking preferably is reversible, which allows for the polymer to linearize or dissociate under appropriate conditions. In some aspects of the present methods, reversible cross-linking may also allow for reversible attachment of a material bound to the surface of a gel bead.

In some aspects, disulfide linkages can be formed between molecular precursor units (e.g., monomers, oligomers, or linear polymers) incorporated into the gel bead. For example, cystamine and modified cystamines are organic agents comprising a disulfide bond that may be used as a crosslinker agent between individual monomeric or polymeric precursors of a gel bead. Polyacrylamide may be polymerized in the presence of cystamine or a species comprising cystamine to generate polyacrylamide gel beads comprising disulfide linkages; that is, chemically degradable beads comprising chemically-reducible cross-linkers. The disulfide linkages permit the bead to be degraded (or dissolved) upon exposure of the bead to a reducing agent.

Functionalization of beads for, e.g., attachment of the barcoded cassette capture primers, barcoded random capture primers or barcoded template switching oligonucleotides or other moieties to the gel beads may be achieved through a number of different approaches, including activation of chemical groups within a polymer, incorporation of active functional groups in the polymer structure, or attachment at the pre-polymer or monomer stage in gel bead production.

The porosity of gel beads can be controlled by adjusting the polymer concentration or degree of crosslinking, effectively creating a tunable molecular cut-off size for transport through the gel. The porosity can then be adjusted to physically retain large molecules of interest while allowing smaller molecules or buffers to be freely exchanged. Alternatively, the polymer network may be chemically modified to conjugate specifically with a target molecule for retention. Also, encapsulated cells, reagents and molecules may be released from a gel bead upon degradation of the gel bead.

FIG. 8A at left shows beads 820 in solution where each bead comprises, e.g., a template switching oligonucleotide (TSO) comprising from 5′ to 3′: a sequence for binding sequencing primers, a barcode specific to each bead (e.g., where the barcode of each bead is different), a unique molecular identifier (where there are many unique molecular identifiers per bead) and a template switching sequence comprising, e.g., a poly-dG tract of nucleotides at its 3′ end. Only beads 820 are shown in FIG. 8A but see FIG. 6A for the composition of the bead-bound oligonucleotides.

Beads 820 are delivered into a flow channel comprising an aqueous (buffer) solution. Cells (830) in an enzyme mix 826 are delivered through an intersecting flow channel to combine with the gel beads 820, and the combination of gel beads 820, cells 830 and enzyme mix 826 are then partitioned into droplets (GEMs) 822 by delivery into partitioning oil 828. The cells are provided at a limiting dilution such that 90-99% of the droplets (GEMs) 822 contain no cells while the remainder of the GEMs 824 contain only one cell. Upon GEM formation, the cell is lysed 812 releasing the cell contents including cellular nucleic acids and editing cassette transcripts and the gel bead is dissolved releasing the primers needed to generate DNAs from the cellular nucleic acids and editing cassette transcripts. Once the cell is lysed, DNAs are created 815 using one or more of the processes of priming, reverse transcription or transcription, extension and amplification (again see FIGS. 6A-6F). Following the generation and amplification of DNAs, the DNAs are sequenced 817 and, using the barcodes, DNAs from the cellular nucleic acids (or mRNAs or analyte tags) can be correlated 819 with DNAs from the editing cassette transcripts present in the cell.

FIG. 8B is a simplified diagram of an alternative embodiment of a method for correlating edits and cellular nucleic acid profiles in a population of cells. Instead of using droplet or GEM partitions, the method depicted in FIG. 8B uses solid wall partitions (e.g., wells). At left in FIG. 8B there is a tube containing edited cells 830. The cells 830 are then diluted such that when delivered to a substrate 840 containing wells 832, some wells will have one cell 830 and some wells will have no cells 834. Once deposited in wells 832, cells 830 are lysed 812 and 1) template switching oligonucleotides comprising a sequence for binding sequencing primers, a barcode specific to each well (e.g., where the barcode of each well is different), and a template switching sequence comprising a poly-dG tract of nucleotides at its 3′ end; 2) random capture primers comprising a random hybridization sequence and a priming sequence (or mRNA capture primers or tag capture primers); and 3) cassette capture primers comprising an editing cassette capture sequence and a priming sequence are added to each well 813. DNAs are then created 815 using the processes of priming, transcription, extension and amplification (again see FIGS. 6A-6F). As with FIG. 8A, the DNAs are sequenced 817 and, using the barcodes, DNAs from the cellular nucleic acids (or mRNAs or analyte tags) can be correlated 819 with DNAs from the editing cassette transcripts.

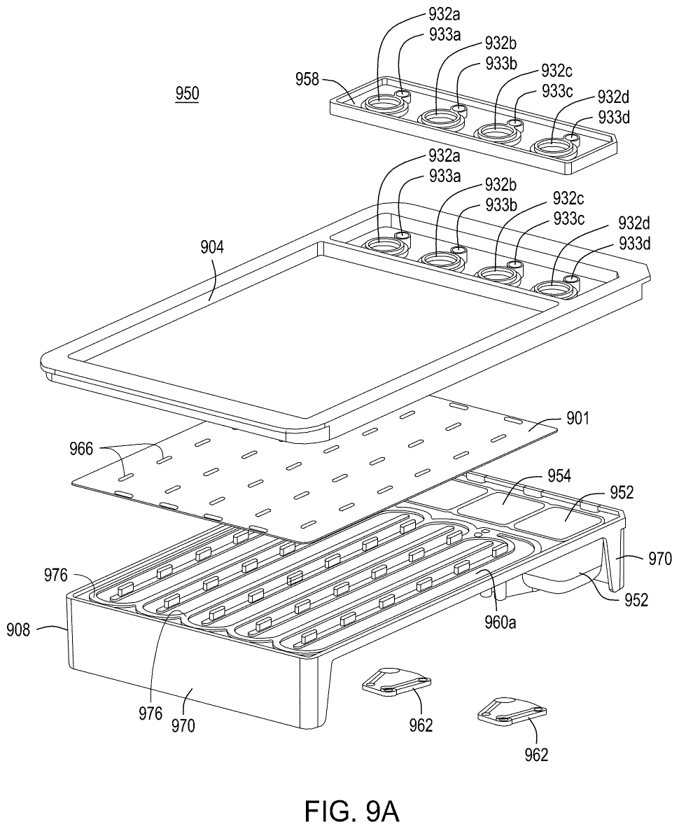

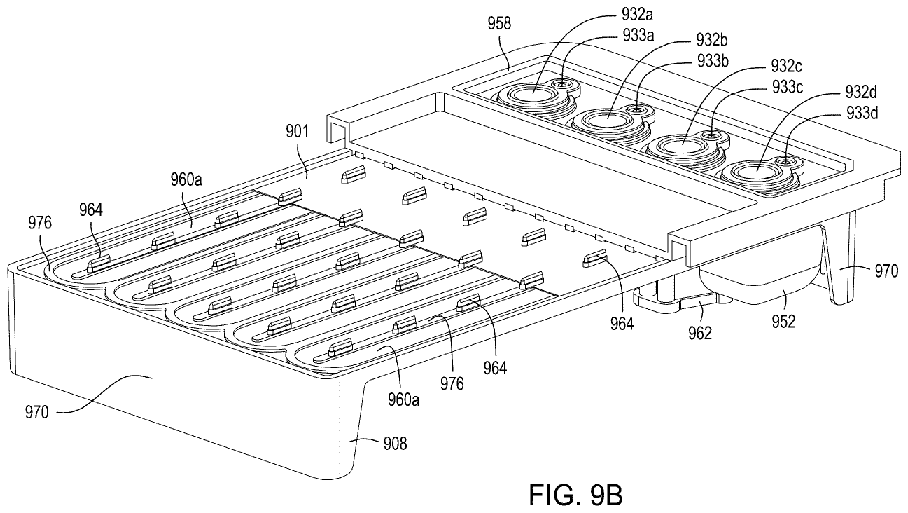

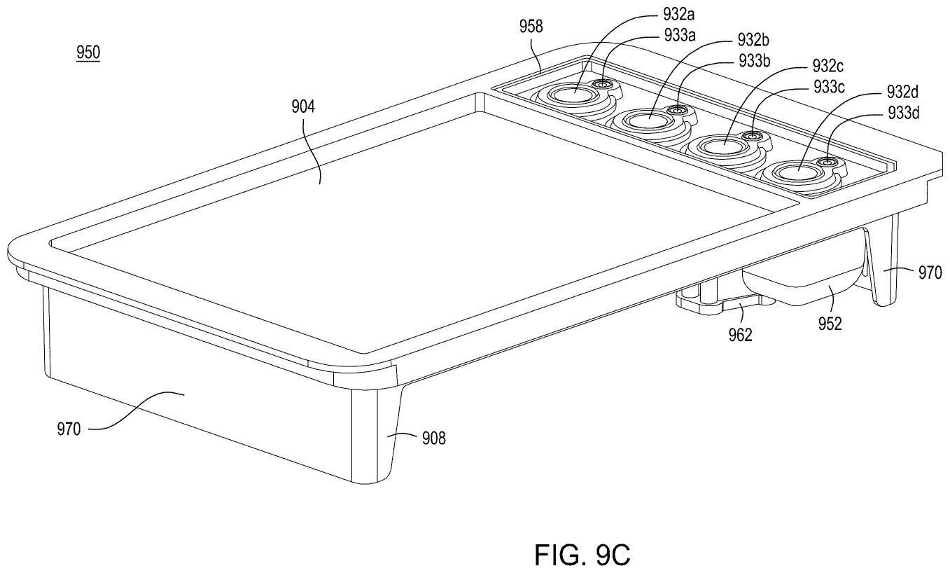

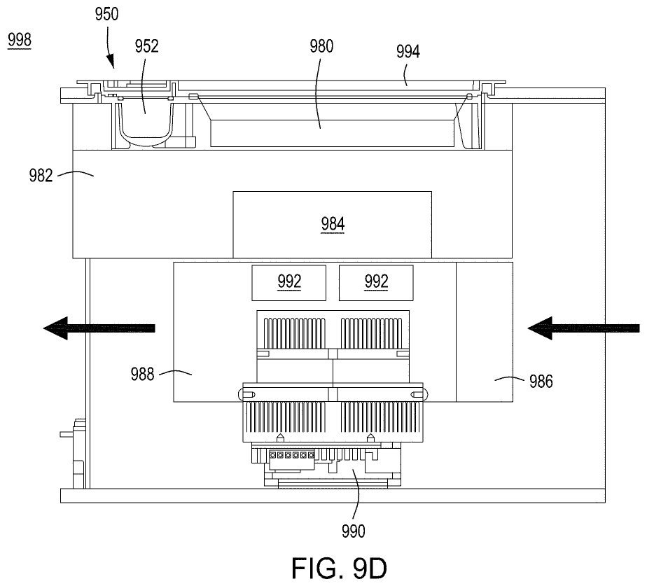

A module useful for performing singulation methods in a solid wall device as depicted in FIG. 8B is a solid wall isolation, incubation, and normalization (SWIIN) module. FIG. 9A depicts an embodiment of a SWIIN module 950 from an exploded top perspective view. In SWIIN module 950 the retentate member is formed on the bottom of a top of a SWIIN module component and the permeate member is formed on the top of the bottom of a SWIIN module component. The SWIIN module 950 in FIG. 9A comprises from the top down, a reservoir gasket or cover 958, a retentate member 904 (where a retentate flow channel cannot be seen in this FIG. 9A), a perforated member 901 swaged with a filter (filter not seen in FIG. 9A), a permeate member 908 comprising integrated reservoirs (permeate reservoirs 952 and retentate reservoirs 954), and two reservoir seals 962, which seal the bottom of permeate reservoirs 952 and retentate reservoirs 954. A permeate channel 960a can be seen disposed on the top of permeate member 908, defined by a raised portion 976 of serpentine channel 960a, and ultrasonic tabs (labeled 964 in FIG. 9B) can be seen disposed on the top of permeate member 908 as well. The perforations that form the wells on perforated member 901 are not seen in this FIG. 9A; however, through-holes 966 to accommodate the ultrasonic tabs 964 are seen. In addition, supports 970 are disposed at either end of SWIIN module 950 to support SWIIN module 950 and to elevate permeate member 908 and retentate member 904 above reservoirs 952 and 954 to minimize bubbles or air entering the fluid path from the permeate reservoir to serpentine channel 960a or the fluid path from the retentate reservoir to serpentine channel 960b (only fluid path 960a is seen in FIG. 9A-9D).

In this FIG. 9A, it can be seen that the serpentine channel 960a that is disposed on the top of permeate member 908 traverses permeate member 908 for most of the length of permeate member 908 except for the portion of permeate member 908 that comprises permeate reservoirs 952 and retentate reservoirs 954 and for most of the width of permeate member 908. As used herein with respect to the distribution channels in the retentate member or permeate member, “most of the length” means about 95% of the length of the retentate member or permeate member, or about 90%, 85%, 80%, 75%, or 70% of the length of the retentate member or permeate member. As used herein with respect to the distribution channels in the retentate member or permeate member, “most of the width” means about 95% of the width of the retentate member or permeate member, or about 90%, 85%, 80%, 75%, or 70% of the width of the retentate member or permeate member.

In this embodiment of a SWIIN module, the perforated member includes through-holes to accommodate ultrasonic tabs disposed on the permeate member. Thus, in this embodiment the perforated member is fabricated from 316 stainless steel, and the perforations form the walls of microwells while a filter or membrane is used to form the bottom of the microwells. Typically, the perforations (microwells) are approximately 150 μm-200 μm in diameter, and the perforated member is approximately 125 μm deep, resulting in microwells having a volume of approximately 2.5 nl, with a total of approximately 200,000 microwells. The distance between the microwells is approximately 279 μm center-to-center. Though here the microwells have a volume of approximately 2.5 nl, the volume of the microwells may be from 1 to 25 nl, or preferably from 2 to 10 nl, and even more preferably from 2 to 4 nl. As for the filter or membrane, like the filter described previously, filters appropriate for use are solvent resistant, contamination free during filtration, and are able to retain the types and sizes of cells of interest. For example, in order to retain small cell types such as bacterial cells, pore sizes can be as low as 0.10 μm, however for other cell types (e.g., such as for mammalian cells), the pore sizes can be as high as 10.0 μm-20.0 μm or more. Indeed, the pore sizes useful in the cell concentration device/module include filters with sizes from 0.10 μm, 0.11 μm, 0.12 μm, 0.13 μm, 0.14 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable material including cellulose mixed ester (cellulose nitrate and acetate) (CME), polycarbonate (PC), polyvinylidene fluoride (PVDF), polyethersulfone (PES), polytetrafluoroethylene (PTFE), nylon, or glass fiber.

The cross-section configuration of the mated serpentine channel may be round, elliptical, oval, square, rectangular, trapezoidal, or irregular. If square, rectangular, or another shape with generally straight sides, the cross section may be from about 2 mm to 15 mm wide, or from 3 mm to 12 mm wide, or from 5 mm to 10 mm wide. If the cross section of the mated serpentine channel is generally round, oval or elliptical, the radius of the channel may be from about 3 mm to 20 mm in hydraulic radius, or from 5 mm to 15 mm in hydraulic radius, or from 8 mm to 12 mm in hydraulic radius.

Serpentine channels 960a and 960b can have approximately the same volume or a different volume. For example, each “side” or portion 960a, 960b of the serpentine channel may have a volume of, e.g., 2 mL, or serpentine channel 960a of permeate member 908 may have a volume of 2 mL, and the serpentine channel 960b of retentate member 904 may have a volume of, e.g., 3 mL. The volume of fluid in the serpentine channel may range from about 2 mL to about 80 mL, or about 4 mL to 60 mL, or from 5 mL to 40 mL, or from 6 mL to 20 mL (note these volumes apply to a SWIIN module comprising a, e.g., 50-500K perforation member). The volume of the reservoirs may range from 5 mL to 50 mL, or from 7 mL to 40 mL, or from 8 mL to 30 mL or from 10 mL to 20 mL, and the volumes of all reservoirs may be the same or the volumes of the reservoirs may differ (e.g., the volume of the permeate reservoirs is greater than that of the retentate reservoirs).

The serpentine channel portions 960a and 960b of the permeate member 908 and retentate member 904, respectively, are approximately 200 mm long, 130 mm wide, and 4 mm thick, though in other embodiments, the retentate and permeate members can be from 75 mm to 400 mm in length, or from 100 mm to 300 mm in length, or from 150 mm to 250 mm in length; from 50 mm to 250 mm in width, or from 75 mm to 200 mm in width, or from 100 mm to 150 mm in width; and from 2 mm to 15 mm in thickness, or from 4 mm to 10 mm in thickness, or from 5 mm to 8 mm in thickness. Embodiments the retentate (and permeate) members may be fabricated from PMMA (poly(methyl methacrylate) or other materials may be used, including polycarbonate, cyclic olefin co-polymer (COC), glass, polyvinyl chloride, polyethylene, polyamide, polypropylene, polysulfone, polyurethane, and co-polymers of these and other polymers. Preferably at least the retentate member is fabricated from a transparent material so that the cells can be visualized (see, e.g., FIG. 9D and the description thereof). For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, Calif.)) obviate the need to use fluorescence, although fluorescent cell markers, fluorescent proteins, and chemiluminescent cell markers may also be used.

Because the retentate member preferably is transparent, colony growth in the SWIIN module can be monitored by automated devices such as those sold by JoVE (ScanLag™ system, Cambridge, Mass.) (also see Levin-Reisman, et al., Nature Methods, 7:737-39 (2010)). Cell growth for, e.g., mammalian cells may be monitored by, e.g., the growth monitor sold by IncuCyte (Ann Arbor, Mich.) (see also, Choudhry, PLos One, 11(2):e0148469 (2016)). Further, automated colony pickers may be employed, such as those sold by, e.g., TECAN (Pickolo™ system, Mannedorf, Switzerland); Hudson Inc. (RapidPick™, Springfield, N.J.); Molecular Devices (QPix 400™ system, San Jose, Calif.); and Singer Instruments (PIXL™ system, Somerset, UK).

Due to the heating and cooling of the SWIIN module, condensation may accumulate on the retentate member which may interfere with accurate visualization of the growing cell colonies. Condensation of the SWIIN module 950 may be controlled by, e.g., moving heated air over the top of (e.g., retentate member) of the SWIIN module 950, or by applying a transparent heated lid over at least the serpentine channel portion 960b of the retentate member 904. See, e.g., FIG. 9D and the description thereof infra.

In SWIIN module 950 cells and medium—at a dilution appropriate for Poisson or substantial Poisson distribution of the cells in the microwells of the perforated member—are flowed into serpentine channel 960b from ports in retentate member 904, and the cells settle in the microwells while the medium passes through the filter into serpentine channel 960a in permeate member 908. The cells are retained in the microwells of perforated member 901 as the cells cannot travel through filter 903 (not shown). Appropriate medium may be introduced into permeate member 908 through permeate ports 911 (not shown). The medium flows upward through filter a to nourish the cells in the microwells (perforations) of perforated member 901. Additionally, buffer exchange can be effected by cycling medium through the retentate and permeate members. In operation, the cells are deposited into the microwells, are grown for an initial, e.g., 2-100 doublings, editing is induced by, e.g., raising the temperature of the SWIIN to 42° C. to induce a temperature inducible promoter or by removing growth medium from the permeate member and replacing the growth medium with a medium comprising a chemical component that induces an inducible promoter.

Once editing has taken place, the temperature of the SWIIN may be decreased, or the inducing medium may be removed and replaced with fresh medium lacking the chemical component thereby de-activating the inducible promoter. The cells then continue to grow in the SWIIN module 950 until the growth of the cell colonies in the microwells is normalized. For the normalization protocol, once the colonies are normalized, the colonies are flushed from the microwells by applying fluid or air pressure (or both) to the permeate member serpentine channel 960a and thus to filter 903 and pooled. Alternatively, if cherry picking is desired, the growth of the cell colonies in the microwells is monitored, and slow-growing colonies are directly selected; or, fast-growing colonies are eliminated.

FIG. 9B is a top perspective view of a SWIIN module with the retentate and perforated members in partial cross section. In this FIG. 9B, it can be seen that serpentine channel 960a is disposed on the top of permeate member 908 is defined by raised portions 976 and traverses permeate member 908 for most of the length and width of permeate member 908 except for the portion of permeate member 908 that comprises the permeate and retentate reservoirs (note only one retentate reservoir 952 can be seen). Moving from left to right, reservoir gasket 958 is disposed upon the integrated reservoir cover 978 (cover not seen in this FIG. 9B) of retentate member 904. Gasket 958 comprises reservoir access apertures 932a, 932b, 932c, and 932d, as well as pneumatic ports 933a, 933b, 933c and 933d. Also at the far left end is support 970. Disposed under permeate reservoir 952 can be seen one of two reservoir seals 962. In addition to the retentate member being in cross section, the perforated member 901 and filter 903 (filter 903 is not seen in this FIG. 9B) are in cross section. Note that there are a number of ultrasonic tabs 664 disposed at the right end of SWIIN module 950 and on raised portion 976 which defines the channel turns of serpentine channel 960a, including ultrasonic tabs 964 extending through through-holes 966 of perforated member 901. There is also a support 970 at the end distal reservoirs 952, 954 of permeate member 908.

FIG. 9C is a side perspective view of an assembled SWIIIN module 950, including, from right to left, reservoir gasket 958 disposed upon integrated reservoir cover 978 (not seen) of retentate member 904. Gasket 958 may be fabricated from rubber, silicone, nitrile rubber, polytetrafluoroethylene, a plastic polymer such as polychlorotrifluoroethylene, or other flexible, compressible material. Gasket 958 comprises reservoir access apertures 932a, 932b, 932c, and 932d, as well as pneumatic ports 933a, 933b, 933c and 933d. Also at the far-left end is support 970 of permeate member 908. In addition, permeate reservoir 952 can be seen, as well as one reservoir seal 962. At the far-right end is a second support 970.

Imaging of cell colonies growing in the wells of the SWIIN is desired in most implementations for, e.g., monitoring both cell growth and device performance and imaging is necessary for cherry-picking implementations. Real-time monitoring of cell growth in the SWIIN requires backlighting, retentate plate (top plate) condensation management and a system-level approach to temperature control, air flow, and thermal management. In some implementations, imaging employs a camera or CCD device with sufficient resolution to be able to image individual wells. For example, in some configurations a camera with a 9-pixel pitch is used (that is, there are 9 pixels center-to-center for each well). Processing the images may, in some implementations, utilize reading the images in grayscale, rating each pixel from low to high, where wells with no cells will be brightest (due to full or nearly-full light transmission from the backlight) and wells with cells will be dim (due to cells blocking light transmission from the backlight). After processing the images, thresholding is performed to determine which pixels will be called “bright” or “dim”, spot finding is performed to find bright pixels and arrange them into blocks, and then the spots are arranged on a hexagonal grid of pixels that correspond to the spots. Once arranged, the measure of intensity of each well is extracted, by, e.g., looking at one or more pixels in the middle of the spot, looking at several to many pixels at random or pre-set positions, or averaging X number of pixels in the spot. In addition, background intensity may be subtracted. Thresholding is again used to call each well positive (e.g., containing cells) or negative (e.g., no cells in the well). The imaging information may be used in several ways, including taking images at time points for monitoring cell growth. Monitoring cell growth can be used to, e.g., remove the “muffin tops” of fast-growing cells followed by removal of all cells or removal of cells in “rounds” as described above, or recover cells from specific wells (e.g., slow-growing cell colonies); alternatively, wells containing fast-growing cells can be identified and areas of UV light covering the fast-growing cell colonies can be projected (or rastered with shutters) onto the SWIIN to irradiate or inhibit growth of those cells. Imaging may also be used to assure proper fluid flow in the serpentine channel 960.

FIG. 9D depicts the embodiment of the SWIIN module in FIGS. 9A-9C further comprising a heat management system including a heater and a heated cover. The heater cover facilitates the condensation management that is required for imaging. Assembly 998 comprises a SWIIN module 950 seen lengthwise in cross section, where one permeate reservoir 952 is seen. Disposed immediately upon SWIIN module 950 is cover 994 and disposed immediately below SWIIN module 950 is backlight 980, which allows for imaging. Beneath and adjacent to the backlight and SWIIN module is insulation 982, which is disposed over a heatsink 984. In this FIG. 9D, the fins of the heatsink would be in-out of the page. In addition there is also axial fan 986 and heat sink 988, as well as two thermoelectric coolers 992, and a controller 990 to control the pneumatics, thermoelectric coolers, fan, solenoid valves, etc. The arrows denote cool air coming into the unit and hot air being removed from the unit. It should be noted that control of heating allows for growth of many different types of cells (prokaryotic and eukaryotic) as well as strains of cells that are, e.g., temperature sensitive, etc., and allows use of temperature-sensitive promoters. Temperature control allows for protocols to be adjusted to account for differences in transformation efficiency, cell growth and viability. For more details regarding solid wall isolation incubation and normalization devices see U.S. Pat. No. 10,533,152, issued 14 Jan. 2020; U.S. Pat. No. 10,550,363, issued 4 Feb. 2020; U.S. Pat. No. 10,532,324, issued 14 Jan. 2020; U.S. Pat. No. 10,633,627, issued 28 Apr. 2020; U.S. Pat. No. 10,633,627, issued 28 Apr. 2020; U.S. Pat. No. 10,625,212, issued 21 Apr. 2020; and U.S. Pat. No. 10,647,982, issued 12 May 2020 and U.S. Ser. No. 16/823,269, filed 18 Mar. 2020; Ser. No. 16/820,292, filed 16 Mar. 2020; Ser. No. 16/820,324, filed 16 Mar. 2020; Ser. No. 16/844,339, filed 9 Apr. 2020.

It should be apparent to one of ordinary skill in the art given the present disclosure that the editing process described may be recursive and multiplexed; that is, cells may go through an editing workflow, then the resulting edited culture may go through another (or several or many) rounds of additional editing (e.g., recursive editing) with different editing vectors before correlating edits with cellular nucleic acids, cell surface proteins, intracellular proteins and/or chromatin accessibility. For example, the cells from round 1 of editing may be diluted and an aliquot of the edited cells edited by editing vector A may be combined with editing vector B, an aliquot of the edited cells edited by editing vector A may be combined with editing vector C, an aliquot of the edited cells edited by editing vector A may be combined with editing vector D, and so on for a second round of editing. After round two, an aliquot of each of the double-edited cells may be subjected to a third round of editing, where, e.g., aliquots of each of the AB-, AC-, AD-edited cells are combined with additional editing vectors, such as editing vectors X, Y, and Z. That is that double-edited cells AB may be combined with and edited by vectors X, Y, and Z to produce triple-edited edited cells ABX, ABY, and ABZ; double-edited cells AC may be combined with and edited by vectors X, Y, and Z to produce triple-edited cells ACX, ACY, and ACZ; and double-edited cells AD may be combined with and edited by vectors X, Y, and Z to produce triple-edited cells ADX, ADY, and ADZ, and so on. In this process, many permutations and combinations of edits can be executed, leading to very diverse cell populations and cell libraries. In any recursive process, it is advantageous to “cure” the previous engine and editing vectors (or single engine+editing vector in a single vector system). “Curing” is a process in which one or more vectors used in the prior round of editing is eliminated from the transformed cells.

Curing can be accomplished by, e.g., cleaving the vector(s) using a curing plasmid thereby rendering the editing and/or engine vector (or single, combined engine/editing vector) nonfunctional; diluting the vector(s) in the cell population via cell growth (that is, the more growth cycles the cells go through, the fewer daughter cells will retain the editing or engine vector(s)), or by, e.g., utilizing a heat-sensitive origin of replication on the editing or engine vector (or combined engine+editing vector). The conditions for curing will depend on the mechanism used for curing; that is, in this example, how the curing plasmid cleaves the editing and/or engine vector.

### EXAMPLES

The following examples are put forth so as to provide those of ordinary skill in the art with a complete disclosure and description of how to make and use the present invention and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Growth in the Cell Growth Module

One embodiment of the cell growth device as described herein was used to grow a yeast cell culture which was monitored in real time using an embodiment of the cell growth device described herein. The rotating growth vial/cell growth device was used to measure OD₆₀₀ in real time of yeast s288c cells in YPAD medium. The cells were grown at 30° C. using oscillating rotation and employing a 2-paddle rotating growth vial. OD₆₀₀ 6.0 was reached in 14 hours (data not shown).

### Example II: Cell Concentration

A tangential flow filtration (TFF) module was used successfully to process and perform buffer exchange on yeast cultures. A yeast culture was initially concentrated to approximately 5 ml using two passes through the TFF device in opposite directions. The cells were washed with 50 ml of 1M sorbitol three times, with three passes through the TFF device after each wash. After the third pass of the cells following the last wash with 1M sorbitol, the cells were passed through the TFF device two times, wherein the yeast cell culture was concentrated to approximately 525 μl. Target conductivity (˜10 S/cm) was achieved in approximately 23 minutes utilizing three 50 ml 1M sorbitol washes and three passes through the TFF device for each wash. The volume of the cells was reduced from 20 ml to 525 μl. Recovery of approximately 90% of the cells has been achieved. (Data not shown.)

### Example III: Production and Transformation of Electrocompetent S. cerevisiae

For testing transformation of a flow-through electroporation (FTEP) device in yeast, S. cerevisiae cells were created using the methods as generally set forth in Bergkessel and Guthrie, Methods Enzymol., 529:311-20 (2013). Briefly, YFAP media was inoculated for overnight growth, with 3 ml inoculate to produce 100 ml of cells. Every 100 ml of culture processed resulted in approximately 1 ml of competent cells. Cells were incubated at 30° C. in a shaking incubator until they reached an OD600 of 1.5+/−0.1.

A conditioning buffer was prepared using 100 mM lithium acetate, 10 mM dithiothreitol, and 50 mL of buffer for every 100 mL of cells grown and kept at room temperature. Cells were harvested in 250 ml bottles at 4300 rpm for 3 minutes, and the supernatant removed. The cell pellets were suspended in 100 ml of cold 1 M sorbitol, spun at 4300 rpm for 3 minutes and the supernatant once again removed. The cells were suspended in conditioning buffer, then the suspension transferred into an appropriate flask and shaken at 200 RPM and 30° C. for 30 minutes. The suspensions were transferred to 50 ml conical vials and spun at 4300 rpm for 3 minutes. The supernatant was removed and the pellet resuspended in cold 1 M sorbitol. These steps were repeated three times for a total of three wash-spin-decant steps. The pellet was suspended in sorbitol to a final OD of 150+/−20.

A comparative electroporation experiment was performed to determine the efficiency of transformation of the electrocompetent S. cerevisiae using the FTEP device. The flow rate was controlled with a syringe pump (Harvard apparatus PHD ULTRA™ 4400). The suspension of cells with DNA was loaded into a 1 mL glass syringe (Hamilton 81320 Syringe, PTFE Luer Lock) before mounting on the pump. The output from the function generator was turned on immediately after starting the flow. The processed cells flowed directly into a tube with 1M sorbitol with carbenicillin. Cells were collected until the same volume electroporated in the NEPAGENE™ had been processed, at which point the flow and the output from the function generator were stopped. After a 3-hour recovery in an incubator shaker at 30° C. and 250 rpm, cells were plated to determine the colony forming units (CFUs) that survived electroporation and failed to take up a plasmid and the CFUs that survived electroporation and took up a plasmid. Plates were incubated at 30° C. Yeast colonies are counted after 48-76 hrs.

The flow-through electroporation experiments were benchmarked against 2 mm electroporation cuvettes (Bull dog Bio) using an in vitro high voltage electroporator (NEPAGENE™ ELEPO21). Stock tubes of cell suspensions with DNA were prepared and used for side-to-side experiments with the NEPAGENE™ and the flow-through electroporation. The device showed better transformation and survival of electrocompetent S. cerevisiae at 2.5 kV voltages as compared to the NEPAGENE™ method. Input is total number of cells that were processed (data not shown).

### Example IV: Singulation of Yeast Colonies in a Solid Wall Device

Electrocompetent yeast cells were transformed with a cloned library, an isothermal assembled library, or a process control sgRNA plasmid (escapee surrogate). Electrocompetent Saccharomyces cerevisiae cells were prepared as follows: The afternoon before transformation was to occur, 10 mL of YPAD was inoculated with the selected Saccharomyces cerevisiae strain. The culture was shaken at 250 RPM and 30° C. overnight. The next day, 100 mL of YPAD was added to a 250-mL baffled flask and inoculated with the overnight culture (around 2 mL of overnight culture) until the OD600 reading reached 0.3+/−0.05. The culture was placed in the 30° C. incubator shaking at 250 RPM and allowed to grow for 4-5 hours, with the OD checked every hour. When the culture reached an OD600 of approximately 1.5, 50 mL volumes were poured into two 50-mL conical vials, then centrifuged at 4300 RPM for 2 minutes at room temperature. The supernatant was removed from all 50 ml conical tubes, while avoiding disturbing the cell pellet. 50 mL of a Lithium Acetate/Dithiothreitol solution was added to each conical tube and the pellet was gently resuspended. Both suspensions were transferred to a 250 mL flask and placed in the shaker; then shaken at 30° C. and 200 RPM for 30 minutes.

After incubation was complete, the suspension was transferred to two 50-mL conical vials. The suspensions then were centrifuged at 4300 RPM for 3 minutes, then the supernatant was discarded. Following the lithium acetate/Dithiothreitol treatment step, cold liquids were used and the cells were kept on ice until electroporation. 50 mL of 1 M sorbitol was added and the pellet was resuspended, then centrifuged at 4300 RPM, 3 minutes, 4° C., after which the supernatant was discarded. The 1M sorbitol wash was repeated twice for a total of three washes. 50 uL of 1 M sorbitol was added to one pellet, cells were resuspended, then transferred to the other tube to suspend the second pellet. The volume of the cell suspension was measured and brought to 1 mL with cold 1 M sorbitol. At this point the cells were electrocompetent and could be transformed with a cloned library, an isothermal assembled library, or process control sgRNA plasmids.

In brief, a required number of 2-mm gap electroporation cuvettes were prepared by labeling the cuvettes and then chilling on ice. The appropriate plasmid—or DNA mixture—was added to each corresponding cuvette and placed back on ice. 100 uL of electrocompetent cells was transferred to each labelled cuvette, and each sample was electroporated using appropriate electroporator conditions. 900 uL of room temperature YPAD Sorbitol media was then added to each cuvette. The cell suspension was transferred to a 14 ml culture tube and then shaken at 30° C., 250 RPM for 3 hours. After a 3 hr recovery, 9 ml of YPAD containing the appropriate antibiotic, e.g., geneticin or Hygromycin B, was added. At this point the transformed cells were processed in parallel in the solid wall device and the standard plating protocol, so as to compare “normalization” in the solid wall device with the standard benchtop process. Immediately before the cells were introduced to the permeable-bottom solid wall device, the 0.45 M filter forming the bottom of the microwells was treated with a 0.1% TWEEN solution to effect proper spreading/distribution of the cells into the microwells of the solid wall device. The filters were placed into a Swinnex Filter Holder (47 mm, Millipore®, SX0004700) and 3 ml of a solution with 0.85% NaCl and 0.1% TWEEN was pulled through the solid wall device and filter through using a vacuum. Different TWEEN concentrations were evaluated, and it was determined that for a 47 mm diameter solid wall device with a 0.45 M filter forming the bottom of the microwells, a pre-treatment of the solid wall device+filter with 0.1% TWEEN was preferred (data not shown).

After the 3-hour recovery in YPAD, the transformed cells were diluted and a 3 ml volume of the diluted cells was processed through the TWEEN-treated solid wall device and filter, again using a vacuum. The number of successfully transformed cells was expected to be approximately 1.0E+06 to 1.0E+08, with the goal of loading approximately 10,000 transformed cells into the current 47 mm permeable-bottom solid wall device (having ˜30,000 wells). Serial dilutions of 10⁻¹, 10⁻², and 10⁻³ were prepared, then 100 μL volumes of each of these dilutions were combined with 3 ml 0.85% NaCl, and the samples were loaded onto solid wall devices. Each permeable-bottom solid wall device was then removed from the Swinnex filter holder and transferred to an LB agar plate containing carbenicillin (100 g/ml), chloramphenicol (25 μg/ml) and arabinose (1% final concentration). The solid wall devices were placed metal side “up,” so that the permeable-bottom membrane was touching the surface of the agar such that the nutrients from the plate could travel up through the filter “bottom” of the wells. The solid wall devices on the YPD agar plates were incubated for 2-3 days at 30° C. See, e.g., FIGS. 3C and 3D.

At the end of the incubation the perforated disks and filters (still assembled) were removed from the supporting nutrient source (in this case an agar plate) and were photographed with a focused, “transilluminating” light source so that the number and distribution of loaded microwells on the solid wall device could be assessed (data not shown). To retrieve cells from the permeable-bottom solid wall device, the filter was transferred to a labeled sterile 100 mm petri dish which contained 15 ml of sterile 0.85% NaCl, then the petri dish was placed in a shaking incubator set to 30° C./80 RPM to gently remove the cells from the filter and resuspend the cells in the 0.85% NaCl. The cells were allowed cells to shake for 15 minutes, then were transferred to a sterile tube, e.g., a 50 ml conical centrifuge tube. The OD600 of the cell suspension was measured; at this point the cells can be processed in different ways depending on the purpose of the study. For example, if an ADE2 stop codon mutagenesis library is used, successfully-edited cells should result in colonies with a red color phenotype when the resuspended cells are spread onto YPD agar plates and allowed to grow for 4-7 days. This phenotypic difference allows for a quantification of percentage of edited cells and the extent of normalization of edited and unedited cells.

### Example V: Increased Editing Efficiency

The afternoon before transformation was to occur, 10 mL of YPAD was added to S. cerevisiae cells, and the culture was shaken at 250 rpm at 30° C. overnight. The next day, approximately 2 mL of the overnight culture was added to 100 mL of fresh YPAD in a 250-mL baffled flask and grown until the OD600 reading reached 0.3+/−0.05. The culture was then placed in a 30° C. incubator shaking at 250 rpm and allowed to grow for 4-5 hours, with the OD checked every hour. When the culture reached ˜1.5 OD600, two 50 mL aliquots of the culture were poured into two 50-mL conical vials and centrifuged at 4300 rpm for 2 minutes at room temperature. The supernatant was removed from the 50 mL conical tubes, avoiding disturbing the cell pellet. 25 mL of lithium acetate/DTT solution was added to each conical tube and the pellet was gently resuspended using an inoculating loop, needle, or long toothpick.

Following resuspension, both cell suspensions were transferred to a 250-mL flask and placed in the shaker to shake at 30° C. and 200 rpm for 30 minutes. After incubation was complete, the suspension was transferred to one 50-mL conical tube and centrifuged at 4300 RPM for 3 minutes. The supernatant was then discarded.

From this point on, cold liquids were used and kept on ice until electroporation was complete. 50 mL of 1 M sorbitol was added to the cells and the pellet was resuspended. The cells were centrifuged at 4300 rpm for 3 minutes at 4° C., and the supernatant was discarded. The centrifugation and resuspension steps were repeated for a total of three washes. 50 μL of 1 M sorbitol was then added to one pellet, the cells were resuspended, then this aliquot of cells was transferred to the other tube and the second pellet was resuspended. The approximate volume of the cell suspension was measured, then brought to a 1 mL volume with cold 1 M sorbitol. The cell/sorbitol mixture and transferred into a 2-mm cuvette. The impedance measurement of the cells was measured in the cuvette. At this point the KΩ must be ≥20. If this is not the case the cells should be washed in cold sorbitol two to three additional times.

Transformation was then performed using 500 ng of linear backbone along with 50 ng ADE2 editing cassettes with the competent S. cerevisiae cells. 2 mm electroporation cuvettes were placed on ice and the plasmid/cassette mix was added to each corresponding cuvette. 100 μL of electrocompetent cells were added to each cuvette and the linear backbone and ADE2 cassettes. Three ade2 cassettes were used, ADE2-70, ADE2-80 and ADE2-90. Each sample was electroporated using the following conditions: Poring pulse: 1800V, 5.0 second pulse length, 50.0 msec pulse interval, 1 pulse; Transfer pulse: 100 V, 50.0 msec pulse length, 50.0 msec pulse interval, with 3 pulses. Once the transformation process is complete, 900 μL of room temperature YPAD Sorbitol media was added to each cuvette.

The cells were then transferred and suspended in a 15 mL tube and incubated shaking at 250 RPM at 30° C. for 3 hours. 9 mL of YPAD and 10 μL of G418 1000× stock was added to the 15 mL tube. 10 μL of each transformation dilution was spread onto two 2×YPD—Kan plates, then placed in an incubator at 30° C. Colonies formed in 3 days. The total number of colonies on each plate was multiplied by 1000 to obtain the total transformant yield for each transformation. Red, white, and partially red colonies were counted to quantify the edit rate. Red and partially red colonies indicated editing while white colonies indicated no editing.

### Example VI: Results of Editing Using Different Editing Cassette Architectures

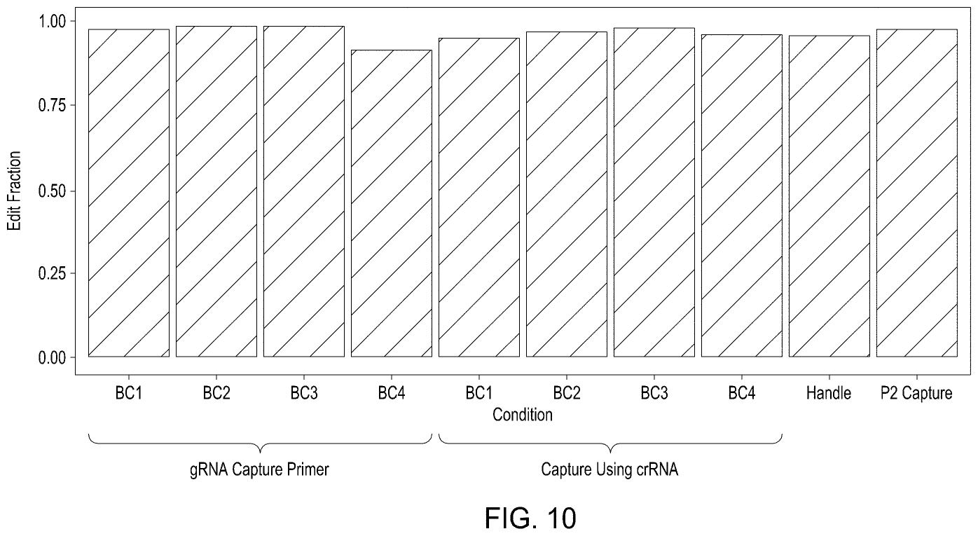

FIG. 10 is a bar graph showing the edit fractions obtained using editing cassettes comprising four different barcodes coupled to an editing cassette (the editing cassette was the same editing cassette in all cases) for the capture sequence capture primer and capture using crRNA architectures, compared to a cassette architecture where a “handle” was added during synthesis of the first cDNA, and using P2 capture (as detailed in FIG. 1C). Note all architectures produced similar high edit fractions.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

We claim:

1. A method for correlating rationally-designed genome edits made in a population of cells with a cellular nucleic acid profile from individual cells comprising:
designing and synthesizing a library of editing cassettes comprising repair templates and gRNAs;
inserting the library of editing cassettes in a vector backbone resulting in a library of editing vectors;
transforming the population of cells with the library of editing vectors to produce transformed cells;
allowing nucleic acid-guided nuclease or nickase fusion editing to take place in the transformed cells to produce edited cells;
singulating the edited cells into partitions;
lysing the edited cells;
adding barcoded product capture primers and barcoded cassette capture primers to each partition, wherein the barcodes used in the barcoded product capture primers and barcoded cassette capture primers in a same partition are a same barcode and wherein the barcodes used in the barcoded product capture primers and barcoded cassette capture primers in a different partition are different from barcodes used in other partitions;
creating DNA copies and/or cDNAs from cellular nucleic acids in the edited cells using the barcoded product capture primers;
creating DNA copies and/or cDNAs from the editing cassettes in the edited cells using the barcoded cassette capture primers;
pooling the DNA copies and/or cDNAs from the partitions;
sequencing the DNA copies and/or cDNAs; and
correlating sequences from the DNA copies and/or cDNAs from cellular nucleic acids with sequences from the DNA copies and/or cDNAs from the editing cassettes for each cell.

2. The method of claim 1, wherein the barcoded product capture primers comprise product capture primers and first barcoded template switching oligonucleotides.

3. The method of claim 2, wherein the barcoded cassette capture primers comprise cassette capture primers and second barcoded template switching oligonucleotides.

4. The method of claim 1, wherein the adding step is performed before singulating the edited cells into the partitions.

5. The method of claim 1, wherein the adding step is performed after singulating the edited cells into the partitions.

6. The method of claim 1, wherein the editing is nucleic acid-guided nuclease editing.

7. The method of claim 1, wherein the editing is nickase fusion editing.

8. The method of claim 1, wherein the population of cells is a population of mammalian cells.

9. The method of claim 8, wherein the vector backbone is a viral vector backbone.

10. The method of claim 9, wherein the viral vector backbone is a lentiviral vector backbone.

11. The method of claim 9, wherein the transforming step comprises transduction.

12. The method of claim 1, wherein the vector backbone further comprises a selection marker and further comprising a selecting step to select for the selection marker after the transforming step.

13. The method of claim 1, wherein the partitions are droplets.

14. The method of claim 1, wherein the partitions are gel beads.

15. The method of claim 1, wherein the partitions are wells.

16. A method for correlating rationally-designed genome edits made in a population of cells with a cellular nucleic acid profile from individual cells comprising:
transforming the population of cells with a coding sequence for a nucleic acid-guided nuclease or a nickase fusion enzyme or a nucleic acid-guided nuclease or a nickase fusion enzyme;
designing and synthesizing a library of editing cassettes comprising repair templates and gRNAs;
inserting the library of editing cassettes in a vector backbone resulting in a library of editing vectors;
transforming the population of cells with the library of editing vectors to produce transformed cells;
allowing nucleic acid-guided nuclease or nickase fusion editing to take place in the transformed cells to produce edited cells;
singulating the edited cells into partitions;
lysing the edited cells;
adding barcoded product capture primers and barcoded cassette capture primers to each partition, wherein the barcodes used in the barcoded product capture primers and barcoded cassette capture primers in a same partition are a same barcode and wherein the barcodes used in the barcoded product capture primers and barcoded cassette capture primers in a different partition are different from barcodes used in other partitions;
creating DNA copies and/or cDNAs from cellular nucleic acids in the edited cells using the barcoded product capture primers;
creating DNA copies and/or cDNAs from the editing cassettes in the edited cells using the barcoded cassette capture primers;
pooling the DNA copies and/or cDNAs from the partitions;
sequencing the DNA copies and/or cDNAs; and
correlating sequences from the DNA copies and/or cDNAs from cellular nucleic acids with sequences from the DNA copies and/or cDNAs from the editing cassettes for each cell.

17. The method of claim 16, wherein the barcoded product capture primers comprise product capture primers and first barcoded template switching oligonucleotides.

18. The method of claim 17, wherein the barcoded cassette capture primers comprise cassette capture primers and second barcoded template switching oligonucleotides.

19. The method of claim 17, wherein the adding step is performed before singulating the edited cells into the partitions.

20. The method of claim 16, wherein the adding step is performed after singulating the edited cells into the partitions.

21. The method of claim 16, wherein the editing is nucleic acid-guided nuclease editing.

22. The method of claim 16, wherein the editing is nickase fusion editing.

23. The method of claim 16, wherein the population of cells is a population of mammalian cells.

24. The method of claim 23, wherein the vector backbone is a viral vector backbone.

25. The method of claim 24, wherein the viral vector backbone is a lentiviral vector backbone.

26. The method of claim 9, wherein the transforming step comprises transduction.

27. The method of claim 1, wherein the vector backbone further comprises a selection marker and further comprising a selecting step to select for the selection marker after the transforming step.

28. The method of claim 1, wherein the partitions are droplets.

29. The method of claim 1, wherein the partitions are gel beads.

30. The method of claim 1, wherein the partitions are wells.
